(19) United States
(12) Patent Application Publication
(10) Pub. No.: US 2003/0138454 A1
(43) Pub. Date: Jul. 24, 2003

(54) Vaccination method

(75) Inventors: Adrian V. S. Hill, Oxford (GB); Helen McShane, Oxford (GB); Sarah C. Gilbert, Oxford (GB); William Reece, Newtown (AU); Joerg Schneider, Barton (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Oxxon Pharmaccines, Ltd., Littlemore (GB)

(21) Appl. No.: 10/079,167

(22) Filed: Feb. 19, 2002

Related U.S. Application Data
(63) Continuation-in-part of application No. 09/454,204, filed on Dec. 9, 1999.
Application No. 09/454,204 is a continuation of application No. PCT/GB98/01681, filed on Jun. 9, 1998.
(63) Continuation-in-part of application No. PCT/GB01/04116, filed on Sep. 13, 2001.

(30) Foreign Application Priority Data
Jun. 9, 1997 (GB) GB9711957.2
Sep. 21, 2000 (GB) GB0023203.3

(51) Int. Cl. G01N 33/53; A61K 39/00; C12N 15/00; C07K 5/00; A61K 38/00; G01N 33/537; A61K 39/38; C12N 15/09; C07K 7/00; A61K 38/04; G01N 33/543; A61K 39/285; A61K 39/04; C12N 15/70; C07K 17/00

(52) U.S. Cl. 424/199.1; 435/320.1; 424/184.1; 424/232.1; 424/277.1; 424/248.1; 424/268.1; 424/273.1; 530/326; 435/7.92

(57) ABSTRACT

New methods and reagents for vaccination are described which generate a CD8 T cell immune response against malarial and other antigens such as viral and tumour antigens. Novel vaccination regimes are described which employ a priming composition and a boosting composition, the boosting composition comprising a non-replicating or replication-impaired pox virus vector carrying at least one CD8 T cell epitope which is also present in the priming composition. There is also provided a method of inducing a CD4+ T-cell response against a target antigen, by administering a composition comprising a source of one or more CD4+ T cell epitopes of the target antigen wherein the source of CD4+ epitopes is a non-replicating or replication impaired recombinant poxvirus vector. A method of inducing a combined CD4+ and CD8+ T cell response against a target antigen is also described herein.

### RELATED APPLICATION(S)

This application is a continuation-in-part of U.S. application Ser. No. 09/454,204, filed Dec. 9, 1999, which is a continuation of International Application No. PCT/GB98/01681, which designated the United States and was filed on Jun. 9, 1998, published in English, and which claims priority under 35 U.S.C. § 119 or 365 to Great Britain, Application No. GB9711957.2, filed Jun. 9, 1997. This application is also a continuation-in-part of PCT/GB01/04116, which designated the United States and was filed on Sep. 13, 2001, which will be published in English, and which claims priority under 35 U.S.C. § 119 or 365 to Great Britain, Application No. GB0023203.3, filed Sep. 21, 2000.

The entire teachings of the above applications are incorporated herein by reference.

### BACKGROUND OF THE INVENTION

Much attention has been focused on inducing CD8+ T cells that may be cytolytic and have been found to protect against some viral infections. In contrast CD4+ T cells have, until recently, usually been regarded as helper T cells that play a role in helping other immunocytes to generate protection, for example by amplifying antibody responses.

However, recent evidence has shown that CD4+ T cells can also be effector cells that play a more direct role in protection. For example in the case of tuberculosis, malaria and H. pylori infection there is evidence for a protective role of CD4 T cells that can secrete the cytokine, gamma-interferon

There is thus a need for the development of vaccines which are capable of stimulating an effective CD4+ T cell response. Poxviruses are known to be good inducers of CD8 T cell responses because of their intracytoplasmic expression. However, they are generally believed to be poor at generating CD4 MHC class II restricted T cells (see for example Haslett et al. Journal of Infectious Diseases 181:1264-72 (2000), page 1268).

Tuberculosis

More than one hundred years after Koch's discovery of the causative organism, tuberculosis remains a major global public health problem. There are estimated to be 8-10 million new cases per annum and the annual mortality is approximately 3 million. The variability in protective efficacy of the currently available vaccine, Mycobacterium bovis bacillus Calmette-Guérin (BCG) (Fine, P. E. and Rodrigues, L. C., Lancet (1990) 335: 1016-1020), and the advent of multi-drug resistant strains of tuberculosis, means that there is an urgent need for a better vaccine.

M. tuberculosis is an intracellular pathogen and the predominant immune response involves the cellular arm of the immune system. There is strong evidence from animal and human studies that CD4+ T cells are necessary for the development of protective immunity (Orme, I. M., J.Immunol. (1987) 138: 293-298; Barnes, P. F. et al., N.Engl.J.Med. (1991) 324: 1644-1650). There is also increasing evidence that CD8+ T cells may play a role (Flynn, J. L., et al., Proc.Natl.Acad.Sci. U.S.A. (1992) 89: 12013-12017; Lalvani, A., et al., Proc.Natl.Acad.Sci. U.S.A. 1998. 95: 270-275.).

DNA vaccines are inducers of cellular immune responses, inducing both CD4+ and CD8+ T cells, and therefore represent a promising delivery system for a tuberculosis vaccine. A number of studies assessing the protective efficacy of DNA vaccines encoding a variety of antigens from M. tuberculosis have shown partial protection against challenge that is equivalent to the protection conferred by BCG (Tascon, et al., Nat.Med. (1996) 2: 888-892; Huygen, et al., Nat.Med. (1996) 2: 893-898). However, none of the vaccine candidates tested so far has been found to be consistently superior to BCG. Although DNA vaccines are good at eliciting both CD4+ and CD8+ T cells, the frequency of response cells they produce may need to be significantly increased in order to confer protection against challenge.

There is thus a need for alternative and improved vaccines capable of inducing a CD4+ T cell response, optionally in conjunction with a CD8+ T cell response for protection against diseases such as tuberculosis.

### SUMMARY OF THE INVENTION

It has now been discovered that non-replicating and replication-impaired strains of poxvirus provide vectors which give an extremely good boosting effect to a primed CTL response. Remarkably, this effect is significantly stronger than a boosting effect by wild type poxviruses. The effect is observed with malarial and other antigens such as viral and tumor antigens, and is protective as shown in mice and non-human primate challenge experiments. Complete rather than partial protection from sporozoite challenge has been observed with the novel immunization regime.

As described herein, replication-defective pox viruses are capable of inducing effector CD4+ T cells that are protective. As shown herein using a gamma-interferon ELISPOT assays with cell subset depletion, they have proved that these CD4+ effector T cells are well induced in both mice and humans after immunisation. The use of heterologous prime-boost regimes with replication-impaired poxviruses induces strong CD4 T cell responses.

Thus the present invention provides a method of inducing a CD4+ T-cell response against a target antigen, which comprises the step of administering at least one dose of:

(a) a first composition comprising a source of one or more CD4+ T cell epitopes of the target antigen;

and at least one dose of

(b) a second composition comprising a source of one or more CD4+ T cell epitopes of the target antigen, including at least one CD4+ T cell epitope which is the same as a CD4+ T cell epitope of the first composition, wherein the source of CD4+ epitopes is a non-replicating or replication impaired recombinant poxvirus vector;

wherein the doses of the first and second compositions may be administered in either order.

If the vector also provides a source of CD8+ T-cell epitopes, then the method of the present invention may induce a combined CD4+/CD8+ T-cell response. Accordingly, the present invention also relates to a method inducing a combined CD4+ and CD8+ T-cell response against a target antigen in a mammal, which comprises the step of administering at least one dose of:

(a) a first composition comprising a source of one or more CD4+ T cell epitopes and a source of one or more CD8+ T cell epitopes of the target antigen;

and at least one dose of

(b) a second composition comprising

(i) a source of one or more CD4+ T cell epitopes of the target antigen, including at least one CD4+ T cell epitope which is the same as a CD4+ T cell epitope of the first composition; and

(ii) a source of one or more CD8+ T cell epitopes of the target antigen, including at least one CD8+ T cell epitope which is the same as a CD8+ T cell epitope of the first composition

wherein the source of CD4+ and CD8+ epitopes for the first and/or second composition is a non-replicating or replication impaired recombinant poxvirus vector; and wherein the doses of the first and second compositions may be administered in either order.

Preferably, if the source of epitopes in (a) is a viral vector, the viral vector in (b) is derived from a different virus.

The first and second compositions used in the method of the present invention may conveniently be provided in the form of a kit. Thus, the present invention also provides a product containing the first and second compositions as a combined preparation for simultaneous, separate or sequential use for inducing CD4+ and/or CD8+ T-cell response against a target antigen.

The present invention also provides the use of such a product in the manufacture of a medicament for inducing CD4+ T-cell response against a target antigen.

The present invention also relates to a method of inducing a CD4+ T-cell response against tuberculosis in a mammal, which comprises the step of administering at least one dose of: (a) a first composition comprising a source of one or more CD4+ T cell epitopes of tuberculosis; and at least one dose of (b) a second composition comprising a source of one or more CD4+ T cell epitopes of tuberculosis, including at least one CD4+ T cell epitope which is the same as a CD4+ T cell epitope of the first composition. In the method, the source of CD4+ epitopes for the first and/or second composition is a non-replicating or replication impaired recombinant poxvirus vector, and the doses of the first and second compositions may be administered in either order. Also encompassed by the present invention is a method of inducing a combined CD4+ and CD8+ T-cell response against tuberculosis in a mammal. The method comprises administering at least one dose of: (a) a first composition comprising a source of one or more CD4+ T cell epitopes and a source of one or more CD8+ T cell epitopes of tuberculosis; and at least one dose of (b) a second composition comprising (i) a source of one or more CD4+ T cell epitopes of tuberculosis, including at least one CD4+ T cell epitope which is the same as a CD4+ T cell epitope of the first composition; and (ii) a source of one or more CD8+ T cell epitopes of the target antigen, including at least one CD8+ T cell epitope which is the same as a CD8+ T cell epitope of the first composition. The source of CD4+ and CD8+ epitopes for the first and/or second composition is a non-replicating or replication impaired recombinant poxvirus vector; and the doses of the first and second compositions may be administered in either order.

A method of inducing a CD4+ T-cell response against malaria in a mammal, which comprises the step of administering at least one dose of (a) a first composition comprising a source of one or more CD4+ T cell epitopes of malaria; and at least one dose of (b) a second composition comprising a source of one or more CD4+ T cell epitopes of malaria, including at least one CD4+ T cell epitope which is the same as a CD4+ T cell epitope of the first composition, is also encompassed by the present invention. The source of CD4+ epitopes for the first and/or second composition is a non-replicating or replication impaired recombinant poxvirus vector, and the doses of the first and second compositions may be administered in either order. In one embodiment, the mammal is a human. The present invention also relates to a method inducing a combined CD4+ and CD8+ T-cell response against malaria in a mammal. The method comprises the step of administering at least one dose of (a) a first composition comprising a source of one or more CD4+ T cell epitopes and a source of one or more CD8+ T cell epitopes of malaria; and at least one dose of (b) a second composition comprising (i) a source of one or more CD4+ T cell epitopes of malaria, including at least one CD4+ T cell epitope which is the same as a CD4+ T cell epitope of the first composition; and (ii) a source of one or more CD8+ T cell epitopes of malaria, including at least one CD8+ T cell epitope which is the same as a CD8+ T cell epitope of the first composition. The source of CD4+ and CD8+ epitopes for the first and/or second composition is a non-replicating or replication impaired recombinant poxvirus vector; and the doses of the first and second compositions may be administered in either order. In one embodiment, the mammal is a human.

A method of inducing a CD4+ T-cell response against human immunodeficiency virus (HIV) in a mammal, which comprises the step of administering at least one dose of (a) a first composition comprising a source of one or more CD4+ T cell epitopes of HIV; and at least one dose of (b) a second composition comprising a source of one or more CD4+ T cell epitopes of HIV, including at least one CD4+ T cell epitope which is the same as a CD4+ T cell epitope of the first composition, is also encompassed by the present invention. The source of CD4+ epitopes for the first and/or second composition is a non-replicating or replication impaired recombinant poxvirus vector, and the doses of the first and second compositions may be administered in either order. In one embodiment, the mammal is a human. The present invention also relates to a method of inducing a combined CD4+ and CD8+ T-cell response against HIV in a mammal. The method comprises the step of administering at least one dose of (a) a first composition comprising a source of one or more CD4+ T cell epitopes and a source of one or more CD8+ T cell epitopes of HIV; and at least one dose of (b) a second composition comprising (i) a source of one or more CD4+ T cell epitopes of HIV, including at least one CD4+ T cell epitope which is the same as a CD4+ T cell epitope of the first composition; and (ii) a source of one or more CD8+ T cell epitopes of HIV, including at least one CD8+ T cell epitope which is the same as a CD8+ T cell epitope of the first composition. The source of CD4+ and CD8+ epitopes for the first and/or second composition is a non-replicating or replication impaired recombinant poxvirus vector; and the doses of the first and second compositions may be administered in either order. In one embodiment, the mammal is a human.

In addition to heterologous prime-boost regimes, the present inventors have shown that replication-defective pox viruses are capable of acting as boosting agents for pre-existing CD4+ T cell responses and combined CD4+ and CD8+ T cell responses. Thus the present invention also provides a medicament for boosting a primed CD4+ T cell response against at least one target antigen, comprising a “second composition” as previously defined. The present invention also provides a method of boosting a primed CD4+ T cell response by administration of such a medicament, and the use of a recombinant non-replicating or replication-impaired pox virus vector in the manufacture of a medicament for boosting a CD4+ T cell immune response. Also encompassed by the present invention is a method of boosting a primed CD4+ and CD8+ T cell response against at least one target antigen in a mammal, which comprises administering a source of one or more CD4+ and CD8+ T cell epitopes of the target antigen, wherein the source of CD4+ and CD8+ T cell epitopes is a non-replicating or a replication-impaired recombinant poxvirus vector.

The present invention also includes a product which comprises (a) a first composition comprising a source of one or more CD4+ T cell epitopes of a target antigen; and (b) a second composition comprising a source of one or more CD4+ T cell epitopes of the target antigen, including at least one CD4+ T cell epitope which is the same as a CD4+ T cell epitope of the first composition. The source of CD4+ epitopes for the first and/or second composition is a non-replicating or replication impaired recombinant poxvirus vector; and the first composition and the second composition are a combined preparation for simultaneous, separate or sequential use for inducing a CD4+ T-cell response against a target antigen. Also encompassed is a product which comprises (a) a first composition comprising a source of one or more CD4+ T cell epitopes and a source of one or more CD8+ T cell epitopes of a target antigen; and (b) a second composition comprising (i) a source of one or more CD4+ T cell epitopes of the target antigen, including at least one CD4+ T cell epitope which is the same as a CD4+ T cell epitope of the first composition; and (ii) a source of one or more CD8+ T cell epitopes of the target antigen, including at least one CD8+ T cell epitope which is the same as a CD8+ T cell epitope of the first composition. The source of CD4+ and CD8+ epitopes for the first and/or second composition is a non-replicating or replication impaired recombinant poxvirus vector; and the first composition and the second composition are a combined preparation for simultaneous, separate or sequential use for inducing a combined CD4+/CD8+ T cell immune response against the target antigen.

The capacity of recombinant replication-impaired poxvirus vectors to induce such functional CD4+ T cell responses, both when used alone and in prime-boost combinations, in both animals and in man, has widespread utility both for prophylactic and for therapeutic vaccination. Such applications include but are not limited to prophylactic vaccination against tuberculosis, HIV, malaria. H. pylori, influenza, hepatitis, CMV, herpes virus-induced diseases and other viral infections, leprosy, non-malarial protozoan parasites such as toxoplasma, and various malignancies, and to therapeutic vaccination against tuberculosis, persistent viral infections such as HIV and chronic hepatitis B and C and many malignancies.

### BRIEF DESCRIPTION OF THE DRAWINGS

FIG. 1 shows the construct used to express Ty-VLP with the malaria epitope cassette CABDHFE. CTL epitopes are from P. falciparum STARP (sporozoite threonine- and asparagine-rich protein) (st), LSA-1 (liver stage antigen 1) (1s), CSP (circumsporozoite protein) (cp), TRAP (thrombospondin-related adhesive protein) (tr), LSA-3 (liver stage antigen 3) (1a) and Exp-1 (exported protein 1) (ex). Helper epitopes are from the P. falciparum CS protein, the M. tuberculosis 38 Kd antigen and Tetanus Toxoid. NANP is the antibody epitope from CS and AM is the adhesion motif from P. falciparum TRAP (Muller et al, EMBO-J., 12:2881-9 (1993)). The length of the complete string is 229 amino acids.

FIG. 2 shows a schematic outline of the H, M and HM proteins. The bar patterns on the schematic representations of the polyepitope proteins indicate the origin of the sequences. The positions of individual epitopes and their MHC restrictions are depicted above and below the proteins. Pb is the only epitope derived from the protein of P. berghei. All other epitopes in the M protein originate from proteins of P. falciparum: cs—circumsporozoite protein, st—STARP, Is—LSA-1 and tr—TRAP. BCG—38 kDa protein of M. tuberculosis; TT—tetanus toxin.

FIG. 3 shows malaria CD8 T cell ELISPOT data following different immunisation regimes. Results are shown as the number of peptide-specific T cells per million splenocytes.

FIGS. 4A-4D show that malaria CD8 T cell ELISPOT (FIGS. 4A and 4C) and CTL levels (FIGS. 4B and 4D) are substantially boosted by a recombinant MVA immunisation following priming with a plasmid DNA encoding the same antigen. The ELISPOT counts are presented on a logarithmic scale.

FIG. 5 shows the CTL responses induced in BALB/c mice to malaria and HIV epitopes by various immunisation regimes employing plasmid DNA and recombinant MVA. Levels of specific lysis at various effector to target ratios are shown.

FIG. 6 shows the results of ELISPOT assays performed to measure the levels of specific CD8+ T cells to the malaria epitope pb9 following different immunisation regimes. Groups of BALB/c mice (n=3) were immunised as indicated (g.g.=gene gun). The time between all immunisations was 14 days. ELISPOT assays were done two weeks after the last immunisation.

FIG. 7 shows the CTL responses against influenza NP in different mouse strains. Mice of different strains were immunised twice two weeks apart with a DNA vaccine V1J-NP encoding for the influenza nucleoprotein (open circles) or primed with the same DNA vaccine and two weeks later boosted with recombinant MVA expressing influenza virus nucleoprotein (closed circles). The CTL activity was determined in a standard ⁵¹Cr-release assay with MHC class I-matched target cells.

FIGS. 8A-8H show CTL responses against different antigens induced in different inbred mouse strains. Mice were immunised with two DNA vaccine immunisations two weeks apart (open circles) or primed with a DNA vaccine and two weeks later boosted with a recombinant MVA expressing the same antigen (closed circles). The strains and antigens were: FIG. 8A, C57BL/6, P. falciparum TRAP;. FIG. 8B, DBA/2, E. coli b-galactosidase; FIG. 8C, BALB/c, HM epitope string CTL activity against malaria peptide (pb9); FIG. 8D, DBA/2, HM epitope string CTL activity against pb9; FIG. 8E, BALB/c;,HM epitope string CTL activity against HIV peptide; FIG. 8F, DBA/2, HM epitope string CTL activity against HIV peptide; FIG. 8G, BALB/c, tumour epitope string CTL activity against P1A-derived peptide; and in FIG. 8H, DBA/2, tumour epitope string CTL activity against P1A-derived peptide. Each curve shows the data for an individual mouse.

FIGS. 9A-9E show sporozoite-primed CTL responses are substantially boosted by MVA. Mice were immunised with: FIG. 9A, two low doses (50+50) of irradiated sporozoites; FIG. 9B,two high doses (300+500) of sporozoites; FIG. 9D, low-dose sporozoite priming followed by boosting with MVA.PbCSP; FIG. 9E, high dose sporozoite priming followed by boosting with MVA.PbCSP. CTL responses following immunisation with MVA.PbCSP are shown in FIG. 9C.

FIGS. 10A and 10B show CTL responses primed by plasmid DNA or recombinant Adenovirus and boosted with MVA. Groups of BALB/c mice (n=3) were primed with plasmid DNA(FIG. 10A) or recombinant Adenovirus expressing β-galactosidase (FIG. 10B). Plasmid DNA was administered intramuscularly, MVA intravenously and Adenovirus intradermally. Splenocytes were restimulated with peptide TPHPARIGL [SEQ ID NO: 69] two weeks after the last immunisation. CTL activity was tested with peptide-pulsed P815 cells.

FIGS. 11A-11C show CTL responses in BALB/c mice primed with plasmid DNA followed by boosting with different recombinant vaccinia viruses. Animals were primed with pTH.PbCSP 50 μg/mouse i.m. and two weeks later boosted with different strains of recombinant vaccina viruses (10⁶ pfu per mouse i.v.) expressing PbCSP. The different recombinant vaccinia virus strains were: FIG. 11A, MVA; FIG. 11B, NYVAC; and WR in Figure C. The frequencies of peptide-specific CD8+ T cells were determined using the ELISPOT assay.

FIG. 12 shows frequencies of peptide-specific CD8+ T cells following different routes of MVA boosting. Results are shown as the number of spot-forming cells (SFC) per one million splenocytes. Each bar represents the mean number of SFCs from three mice assayed individually.

FIG. 13 shows the survival rate of the two groups of mice. Sixty days after challenge eight out of ten mice were alive in the group immunised with the tumour epitopes string.

FIG. 14 shows results of an influenza virus challenge experiment. BALB/c mice were immunised as indicated. GG=gene gun immunisations, im=intramuscular injection, iv=intravenous injection. Survival of the animals was monitored daily after challenge.

FIG. 15 shows detection of SIV-specific MHC class I-restricted CD8+ T cells using tetramers. Each bar represents the percentage of CD8+ T cells specific for the Mamu-A*01/gag epitope at the indicated time point. One percent of CD8 T cells corresponds to about 5000/10⁶ peripheral blood lymphocytes.

FIG. 16 shows CTL induction in macaques following DNA/MVA immunisation. PBMC from three different macaques (CYD, DI and DORIS) were isolated at week 18, 19 and 23 and were restimulated with peptide CTPYDINQM [SEQ ID NO: 54] in vitro. After two restimulations with peptide CTPYDINQM [SEQ ID NO: 54] the cultures were tested for their lytic activity on peptide-pulsed autologous target cells.

FIG. 17 shows two graphs of the efficacy of various immunisation regimes after 8 weeks. Data represent the mean and standard error of 7-15 mice/group.

FIG. 18 is a graph showing the results of a ⁵¹Cr Release assay performed on the splenocytes from mice in the DDDM group.

FIG. 19 is a graph comparing heterologous and homologous regime's protection to challenge. Mean CFU counts/organ were taken at 8 weeks. *, p<0.05; **, p<0.01 when compared to the naive control group.

FIG. 20 is a graph showing that heterologous prime-boost induces stronger responses than homologous vaccination to pool TT1-10. Box plots of the size of the response 7 days after three vaccinations with either homologous (M3) or heterologous (D2M, DM2, G2M) vaccination regimes are shown. Responses shown are ex vivo ELISPOT responses to (a) a pool of peptides spanning the N-terminal 110 amino acids of TRAP strain T9/96.

FIG. 21 shows three graphs of malaria vaccine specific responses in all three donors tested to peptide pool TT1-10 are depleted by the removal of CD4⁺ T cells, but not by CD8⁺ T cells. In this study, PBMC from three donors were tested 7 days after the last immunisation (donors 012 and 028) or 21 days after the last immunisation (donor 049). PBMCs were tested for anti TRAP pool TT1-110 responses (undepleted), PBMCs CD4+ T depleted (CD4) or PBMCs CD8+ T cells depleted (CD8).

FIG. 22 is a graph showing responses to the Tetanus Toxoid epitope FTTp, 7 days after vaccination in heterologous and homologous prime-boost vaccination regimes.

FIG. 23 is a schematic diagram of the melanoma poly-epitope gene Me13. CTL epitopes are denoted by solid lines. The HLA molecules associated with each epitope are shown above the solid lines (A1, A2 for human; D^{b} for murine).

FIG. 24 shows graphs of the kinetics of melan-A/HLA-A2 specific CD8+ T cells expansion detected in the peripheral blood of a vaccinated melanoma patient.

FIG. 25 are three graphs showing effector T cell responses by ex vivo ELISPOT for one volunteer at three timepoints. 13 peptide pools are shown along the x axis, each in duplicate. Pool 1 is the negative control (cells, no peptide). Pools 2-4 span the ME string. Pools 4-9 span 3D7 TRAP (cross-reactive response). Pools 10-13 span T996 TRAP (homologous response).

FIGS. 26A-26D are graphs showing the mean T996- and 3D7-TRAP specific effector T cell frequencies in DNA/MVA vs MVA vaccinated groups in The Gambia and UK. Arithmetic mean effector frequencies (standard error) as follows: FIG. 26A, DNA/MVA group in The Gambia; FIG. 26B, MVA group in The Gambia; FIG. 26C, DNA/MVA group in UK; FIG. 26D, MVA group in UK. Post-DNA in FIGS. 26A and 26C refers to the timepoint after the second DNA vaccination. Post-MVA in FIGS. 26A and 26C refers to the timepoint after the first MVA vaccination in the DNA/MVA groups. Final refers the 8-10 weeks after final vaccination timepoint.

FIGS. 27A-27B are graphs showing effector T Cell subsets induced by DNA/MVA and MVA vaccination in Gambian volunteers. The subset distribution of nine induced effector T cell responses in seven volunteers were evaluated on frozen/thawed cells. Numbers along the x axis are volunteer identification numbers. Each response is specific for one peptide pool consisting of 20-mers spanning a portion of either T996 or 3D7 TRAP (various pools from pools 5-14 in FIG. 25) and was evaluated at the timepoint at which that response was of maximal magnitude.

FIG. 28 is the vaccine construct inserted into both the plasmid pSG2 under the control of the CMV promoter, and into the genome of MVA.

FIG. 29 is a graph showing ex vivo IFNγ ELISPOT responses to peptide pools at prevaccination (d0), and 7 days after either 2 i.m. DNA injections (D2), 2 gene gun injections (G2), 2 DNA followed by 1 MVA (D2M and G2M) or 3 MVA injections (M3). All vaccines were administered three weeks apart.

FIG. 30 is a graph showing ex vivo IFNγ ELISPOT responses to peptide pools after the first and second boosting vaccinations. There was no evidence of boosting with the second MVA vaccination.

FIG. 31 is a graph showing the onset of parasitemia in volunteers that were inoculated by bites from five mosquitoes infected with the heterologous P. falciparum strain 3D7. They were followed daily by thick film microscopy. On first evidence of parasitemia, the volunteers were cured with chloroquine. There was a significant delay in the onset of parasitemia in the G2M2 group, which had the highest immune responses.

FIG. 32 is a schematic representation of the contents of DNA plasmid used in Example 13.

FIG. 33 is a bar graph showing ELISPOT responses to pools of peptides seven days after various vaccination regimens showing summed responses to pools of peptides from all tested, T996 strain of TRAP and 3D7 strain of TRAP. The bracketed numerals included in the regimen names correspond to the dosage of vaccine, as indicated in Table 2. The arithmetic mean of the responses for the subjects in that group are presented with an error bar to indicate the standard error of the mean.

FIG. 34 is a bar graph showing a time course after vaccination is shown at seven, 28 and 150-350 days after vaccination for six subjects in group GGMM(3). The geometric mean and standard error are shown.

FIG. 35 is a graph of the Kaplan-Meier curves of time from sporozoite challenge to parasitaemia detected on thick blood film for 2 groups; 16 unvaccinated control subjects and 14 vaccinated subjects who received either GGMM(3) (n=6), DDDMM(15) (n=4) or DDD_MM(15) (n=4) [see Table 2 for details]. The comparison of vaccinated and unvaccinated is significant in log rank test p=0.0133.

### DETAILED DESCRIPTION OF THE INVENTION

The present invention relates to methods of generating a CD8+, CD4+ and/or a combined CD4+/CD8+ T cell response against a target antigen in a mammal (e.g., primate, particularly human). In one embodiment, the T cell response is a protective immune response.

It is an aim of this invention to identify an effective means of immunizing against malaria. It is a further aim of this invention to identify means of immunizing against other diseases in which CD8+ T cell responses play a protective role. Such diseases include but are not limited to infection and disease caused by the viruses HIV, herpes simplex, herpes zoster, hepatitis C, hepatitis B, influenza, Epstein-Barr virus, measles, dengue and HTLV-1; by the bacteria Mycobacterium tuberculosis and Listeria sp.; and by the protozoan parasites Toxoplasma and Trypanosoma; and certain forms of cancer e.g. melanoma, cancer of the breast and cancer of the colon.

We describe here a novel method of immunizing that generated very high levels of CD8+ T cells and was found to be capable of inducing unprecedented complete protection against P. berghei sporozoite challenge. The same approach was tested in higher primates and found to be highly immunogenic in this species also, and was found to induce partial protection against P. falciparum challenge. Induction of protective immune responses has also been demonstrated in two additional mouse models of viral infection and cancer.

We show further than the novel immunization regime that is described here is also effective in generating strong CD8+ T cell responses against HIV epitopes. Considerable evidence indicates that the generation of such CD8+ T cell responses can be expected to be of value in prophylactic or therapeutic immunization against this viral infection and disease (Gallimore et al., Nature Medicine, 1: 1167-73 (1995); Ada, Journal of Medical Primatology, 25: 158-62 (1996)). We demonstrate that strong CD8+ T cell responses may be generated against epitopes from both HIV and malaria using an epitope string with sequences from both of these micro-organisms. The success in generating enhanced immunogenicity against both HIV and malaria epitopes, and also against influenza and tumor epitopes, indicates that this novel immunization regime can be effective generally against many infectious pathogens and also in non-infectious diseases where the generation of a strong CD8+ T cell response may be of value.

A surprising feature of the current invention is the finding of the very high efficacy of non-replicating agents in both priming and particularly in boosting a CD8+ T cell response. In general the immunogenicity of CD8+ T cell induction by live replicating viral vectors has previously been found to be higher than for non-replicating agents or replication-impaired vectors. This is as would be expected from the greater amount of antigen produced by agents that can replicate in the host. Here however we find that the greatest immunogenicity and protective efficacy is surprisingly observed with non-replicating vectors. The latter have an added advantage for vaccination in that they are in general safer for use in humans than replicating vectors.

The present invention provides in one aspect a kit for generating a protective CD8+ T cell immune response against at least one target antigen, which kit comprises:

(i) a priming composition comprising a source of one or more CD8+ T cell epitopes of the target antigen, together with a pharmaceutically acceptable carrier; and

(ii) a boosting composition comprising a source of one or more CD8+ T cell epitopes of the target antigen, including at least one CD8+ T cell epitope which is the same as a CD8+ T cell epitope of the priming composition, wherein the source of CD8+ T cell epitopes is a non-replicating or replication-impaired recombinant poxvirus vector, together with a pharmaceutically acceptable carrier; with the proviso that if the source of epitopes in (i) is a viral vector, the viral vector in (ii) is derived from a different virus.

In another aspect the invention provides a method for generating a protective CD8+ T cell immune response against at least one target antigen, which method comprises administering at least one dose of component (i), followed by at least one dose of component (ii) of the kit according to the invention.

Preferably, the source of CD8+ T cell epitopes in (i) in the method according to the invention is a non-viral vector or a non-replicating or replication-impaired viral vector, although replicating viral vectors may be used.

Preferably, the source of CD8+ T cell epitopes in (i) is not a poxvirus vector, so that there is minimal cross-reactivity between the primer and the booster.

In one preferred embodiment of the invention, the source of CD8+ T cell epitopes in the priming composition is a nucleic acid, which may be DNA or RNA, in particular a recombinant DNA plasmid. The DNA or RNA may be packaged, for example in a lysosome, or it may be in free form.

In another preferred embodiment of the invention, the source of CD8+ T cell epitopes in the priming composition is a peptide, polypeptide, protein, polyprotein or particle comprising two or more CD8+ T cell epitopes, present in a recombinant string of CD8+ T cell epitopes or in a target antigen. Polyproteins include two or more proteins which may be the same, or preferably different, linked together. Particularly preferred in this embodiment is a recombinant proteinaceous particle such as a Ty virus-like particle (VLP) (Burns et al. Molec. Biotechnol. 1994, 1:137-145).

Preferably, the source of CD8+ T cell epitopes in the boosting composition is a vaccinia virus vector such as MVA or NYVAC. Most preferred is the vaccinia strain modified virus ankara (MVA) or a strain derived therefrom. Alternatives to vaccinia vectors include avipox vectors such as fowlpox or canarypox vectors. Particularly suitable as an avipox vector is a strain of canarypox known as ALVAC (commercially available as Kanapox), and strains derived therefrom.

Poxvirus genomes can carry a large amount of heterologous genetic information. Other requirements for viral vectors for use in vaccines include good immunogenicity and safety. MVA is a replication-impaired vaccinia strain with a good safety record. In most cell types and normal human tissues, MVA does not replicate; limited replication of MVA is observed in a few transformed cell types such as BHK21 cells. It has now been shown, by the results described herein, that recombinant MVA and other non-replicating or replication-impaired strains are surprisingly and significantly better than conventional recombinant vaccinia vectors at generating a protective CD8+ T cell response, when administered in a boosting composition following priming with a DNA plasmid, a recombinant Ty-VLP or a recombinant adenovirus.

It will be evident that vaccinia virus strains derived from MVA, or independently developed strains having the features of MVA which make MVA particularly suitable for use in a vaccine, will also be suitable for use in the invention.

MVA containing an inserted string of epitopes (MVA-HM, which is described in the Examples) has been deposited at the European Collection of Animal Cell Cultures, CAMR, Salisbury, Wiltshire SP4 0JG, UK under accession no. V97060511 on Jun. 5, 1997.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

Replication of a virus is generally measured in two ways: 1) DNA synthesis and 2) viral titre. More precisely, the term “non-replicating or replication-impaired” as used herein and as it applies to poxviruses means viruses which satisfy either or both of the following criteria:

1) exhibit a 1 log (10 fold) reduction in DNA synthesis compared to the Copenhagen strain of vaccinia virus in MRC-5 cells (a human cell line);

2) exhibit a 2 log reduction in viral titre in HELA cells (a human cell line) compared to the Copenhagen strain of vaccinia virus.

Examples of poxviruses which fall within this definition are MVA, NYVAC and avipox viruses, while a virus which falls outside the definition is the attenuated vaccinia strain M7.

Alternative preferred viral vectors for use in the priming composition according to the invention include a variety of different viruses, genetically disabled so as to be non-replicating or replication-impaired. Such viruses include for example non-replicating adenoviruses such as E1 deletion mutants. Genetic disabling of viruses to produce non-replicating or replication-impaired vectors has been widely described in the literature (e.g. McLean et al., J. Infect. Dis., 170(5): 1100-9 (1994)).

Other suitable viral vectors for use in the priming composition are vectors based on herpes virus and Venezuelan equine encephalitis virus (VEE) (Davies et al., J. Virol, 70(6): 3781-7 (1996)). Suitable bacterial vectors for priming include recombinant BCG and recombinant Salmonella and Salmonella transformed with plasmid DNA (Darji A et al. 1997 Cell 91: 765-775).

Alternative suitable non-viral vectors for use in the priming composition include lipid-tailed peptides known as lipopeptides, peptides fused to carrier proteins such as KLH either as fusion proteins or by chemical linkage, whole antigens with adjuvant, and other similar systems. Adjuvants such as QS21 or SBAS2 (Stoute J A et al. 1997 N Engl J Medicine 226: 86-91) may be used with proteins, peptides or nucleic acids to enhance the induction of T cell responses. These systems are sometimes referred to as “immunogens” rather than “vectors”, but they are vectors herein in the sense that they carry the relevant CD8+ T cell epitopes.

There is no reason why the priming and boosting compositions should not be identical in that they may both contain the priming source of CD8+ T cell epitopes as defined in (i) above and the boosting source of CD8+ T cell epitopes as defined in (ii) above. A single formulation which can be used as a primer and as a booster will simplify administration. The important thing is that the primer contains at least the priming source of epitopes as defined in (i) above and the booster contains at least the boosting source of epitopes as defined in (ii) above.

The CD8+ T cell epitopes either present in, or encoded by the priming and boosting compositions, may be provided in a variety of different forms, such as a recombinant string of one or two or more epitopes, or in the context of the native target antigen, or a combination of both of these. CD8+ T cell epitopes have been identified and can be found in the literature, for many different diseases. It is possible to design epitope strings to generate a CD8+ T cell response against any chosen antigen that contains such epitopes. Advantageously, the epitopes in a string of multiple epitopes are linked together without intervening sequences so that unnecessary nucleic acid and/or amino acid material is avoided. In addition to the CD8+ T cell epitopes, it may be preferable to include one or more epitopes recognized by T helper cells, to augment the immune response generated by the epitope string. Particularly suitable T helper cell epitopes are ones which are active in individuals of different HLA types, for example T helper epitopes from tetanus (against which most individuals will already be primed). A useful combination of three T helper epitopes is employed in the examples described herein. It may also be useful to include B cell epitopes for stimulating B cell responses and antibody production.

The priming and boosting compositions described may advantageously comprise an adjuvant. In particular, a priming composition comprising a DNA plasmid vector may also comprise granulocyte macrophage-colony stimulating factor (GM-CSF), or a plasmid encoding it, to act as an adjuvant; beneficial effects are seen using GM-CSF in polypeptide form.

The compositions described herein may be employed as therapeutic or prophylactic vaccines. Whether prophylactic or therapeutic immunization is the more appropriate will usually depend upon the nature of the disease. For example, it is anticipated that cancer will be immunized against therapeutically rather than before it has been diagnosed, while anti-malaria vaccines will preferably, though not necessarily be used as a prophylactic.

The compositions according to the invention may be administered via a variety of different routes. Certain routes may be favoured for certain compositions, as resulting in the generation of a more effective response, or as being less likely to induce side effects, or as being easier for administration. The present invention has been shown to be effective with gene gun delivery, either on gold beads or as a powder.

In further aspects, the invention provides:

a method for generating a protective CD8+ T cell immune response against a pathogen or tumor, which method comprises administering at least one dose of a recombinant DNA plasmid encoding at least one CD8+ T cell epitope or antigen of the pathogen or cancer, followed by at least one dose of a non-replicating or replication-impaired recombinant pox virus encoding the same epitope or antigen;

a method for generating a protective CD8+ T cell immune response against a pathogen or tumor, which method comprises administering at least one dose of a recombinant protein or particle comprising at least one epitope or antigen of the pathogen or cancer, followed by at least one dose of a recombinant MVA vector encoding the same epitope or antigen;

the use of a recombinant non-replicating or replication-impaired pox virus vector in the manufacture of a medicament for boosting a CD8+ T cell immune response;

the use of an MVA vector in the manufacture of a medicament for boosting a CD8+ T cell immune response;

a medicament for boosting a primed CD8+ T cell response against at least one target antigen or epitope, comprising a source of one or more CD8+ T cell epitopes of the target antigen, wherein the source of CD8+ T cell epitopes is a non-replicating or a replication-impaired recombinant poxvirus vector, together with a pharmaceutically acceptable carrier; and

the priming and/or boosting compositions described herein, in particulate form suitable for delivery by a gene gun; and methods of immunization comprising delivering the compositions by means of a gene gun.

Also provided by the invention are: the epitope strings described herein, including epitope strings comprising the amino acid sequences listed in table 1 and table 2; recombinant DNA plasmids encoding the epitope strings; recombinant Ty-VLPs comprising the epitope strings; a recombinant DNA plasmid or non-replicating or replication impaired recombinant pox virus encoding the P. falciparum antigen TRAP; and a recombinant polypeptide comprising a whole or substantially whole protein antigen such as TRAP and a string of two or more epitopes in sequence such as CTL epitopes from malaria.

CD4+ and Combined CD4+/CD8+ Immune Responses

In a first aspect, the present invention relates to a method of inducing a CD4+ T cell response. The method may also coinduce a CD8+ immune response.

T cells fall into two major groups which are distinguishable by their expression of either the CD4 or CD8 co-receptor molecules. CD8-expressing T cells are also known as cytotoxic T cells by virtue of their capacity to kill infected cells. CD4-expressing T cells, on the other hand, have been implicated in mainly “helping” or “inducing” immune responses.

The nature of a T cell immune response can be characterised by virtue of the expression of cell surface markers on the cells. T cells in general can be detected by the present of TCR, CD3, CD2, CD28, CD5 or CD7 (human only). CD4+ T cells and CD8+ T cells can be distinguished by their co-receptor expression (for example, by using anti-CD4 or anti-CD8 monoclonal antibodies, as is described in the Examples).

Since CD4+ T cells recognise antigens when presented by MHC class II molecules, and CD8+ recognise antigens when presented by MHC class I molecules, CD4+ and CD8+ T cells can also be distinguished on the basis of the antigen presenting cells with which they will react.

Within a particular target antigen, there may be one or more CD4+ T cell epitopes and one or more CD8+ T cell epitopes. If the particular epitope has already been characterised, this can be used to distinguish between the two subtypes of T cell, for example on the basis of specific stimulation of the T cell subset which recognises the particular epitope.

CD4+ T cells can also be subdivided on the basis of their cytokine secretion profile. The T_{H}1 subset (sometimes known as “inflammatory CD4 T cells”) characteristically secretes IL-2 and IFNγ and mediates several functions associated with cytotoxicity and local inflammatory reactions. T_{H}1 cells are capable of activating macrophages leading to cell mediated immunity. The T_{H}2 subset (sometimes known as “helper CD4 T cells”) characteristically secretes Il-4, IL-5, IL-6 and IL-10, and is thought to have a role in stimulating B cells to proliferate and produce antibodies (humoral immunity).

T_{H}1 and T_{H}2 cells also have characteristic expression of effector molecules. T_{H}1 cells expressing membrane-bound TNF and T_{H}2 cells expressing CD40 ligand which binds to CD40 on the B cell.

Preferably the CD4+ T cell response induced by the method of the present invention is a TH1-type response. Preferably the induced CD4+ T cells are capable of secreting IFNγ.

The induction of a CD4+ or CD8+ immune response will cause an increase in the number of the relevant T cell type. This may be detected by monitoring the number of cells, or a shift in the overall cell population to reflect an increasing proportion of CD4+ or CD8+ T cells). The number of cells of a particular type may be monitored directly (for example by staining using an anti-CD4/CD8 antibody, and then analysing by fluorescence activated cell scanning (FACScan)) or indirectly by monitoring the production of, for example a characteristic cytokine. In the Examples the presence of CD4+ T cells is monitored on the basis of their capacity to secrete IFNγ, in response to a specific peptide, using an ELISPOT assay. CD4 and CD8 T cell responses are readily distinguished in ELISPOT assays by specific depletion of one or other T cell subset using appropriate antibodies. CD4 and CD8 T cell responses are also readily distinguished by FACS (fluorescence activated cell sorter) analysis.

CD4+/CD8+ T Cell Epitopes

The method comprises the step of administering one or more CD4+ T cell epitopes (optionally with one or more CD8+ T cell epitopes) of a target antigen.

A T cell epitope is a short peptide derivable from a protein antigen. Antigen presenting cells can internalise antigen and process it into short fragments which are capable of binding MHC molecules. The specificity of peptide binding to the MHC depends on specific interactions between the peptide and the peptide-binding groove of the particular MHC molecule.

Peptides which bind to MHC class I molecules (and are recognised by CD8+ T cells) are usually between 6 and 12, more usually between 8 and 10 amino acids in length. The amino-terminal amine group of the peptide makes contact with an invariant site at one end of the peptide groove, and the carboxylate group at the carboxy terminus binds to an invariant site at the other end of the groove. The peptide lies in an extended confirmation along the groove with further contacts between main-chain atoms and conserved amino acid side chains that line the groove. Variations in peptide length are accomodated by a kinking in the peptide backbone, often at proline or glycine residues.

Peptides which bind to MHC class II molecules are usually at least 10 amino acids, more usually at least 13 amino acids in length, and can be much longer. These peptides lie in an extended confirmation along the MHC II peptide-binding groove which is open at both ends. The peptide is held in place mainly by main-chain atom contacts with conserved residues that line the peptide-binding groove.

For a given antigen, CD4+ and CD8+ epitopes may be characterised by a number of methods known in the art. When peptides are purified from cells, their bound peptides co-purify with them. The peptides can then by eluted from the MHC molecules by denaturing the complex in acid, releasing the bound peptide, which can be purified (for example by HPLC) and perhaps sequenced.

Peptide binding to many MHC class I and II molecules has been analysed by elution of bound peptides and by X-ray crystallography. From the sequence of a target antigen, it is possible to predict, to a degree, where the Class I and Class II peptides may lie. This is particularly possible for MHC class I peptides, because peptides that bind to a given allelic variant of an MHC class I molecule have the same or very similar amino acid residues at two or three specific positions along the peptide sequence, known as anchor residues.

Also, it is possible to elucidate CD4+ and CD8+ epitopes using overlapping peptide libraries which span the length of the target antigen. By testing the capacity of such a library to stimulate CD4+ or CD8+ T cells, one can determine the which peptides are capable of acting as T cell epitopes. In the examples, a peptide library for two antigens from M. tuberculosis are analysed using an ELISPOT assay.

Sources of T Cell Epitopes

The method of the present invention is a “prime-boost” administration regime, and involves the administration of at least two compositions:

(a) a first composition comprising a source of one or more CD4+ T cell epitopes of the target antigen; and

(b) a second composition comprising a source of one or more CD4+ T cell epitopes of the target antigen, including at least one CD4+ T cell epitope which is the same as a CD4+ T cell epitope of the first composition, wherein the source of CD4+ epitopes is a non-replicating or replication impaired recombinant poxvirus vector.

The CD4+ and optionally CD8+ T cell epitopes either present in, or encoded by the compositions, may be provided in a variety of different forms; such as a recombinant string of one or two or more epitopes, or in the context of the native target antigen, or a combination of both of these. CD4+ and CD8+ T cell epitopes have been identified and can be found in the literature, for many different diseases. It is possible to design epitope strings to generate a CD4+ and/or CD8+ T cell response against any chosen antigen that contains such epitopes. Advantageously, the epitopes in a string of multiple epitopes are linked together without intervening sequences so that unnecessary nucleic acid and/or amino acid material is avoided. In addition to the CD4+ T cell epitopes from the target antigen, it may be preferable to include one or more other epitopes recognised by T helper cells, to augment the immune response generated by the epitope string. Particularly suitable T helper cell epitopes are ones which are active in individuals of different HLA types, for example T helper epitopes from tetanus (against which most individuals will already be primed).

Preferably, the source of CD4+ (and optionally CD8+) T cell epitopes in the first composition in the method according to the invention is a non-viral vector or a non-replicating or replication-impaired viral vector, although replicating viral vectors may be used, as may different types of poxvirus—for example fowlpox with MVA or the converse.

Preferably, the source of T cell epitopes in the first composition is not a poxvirus vector, so that there is minimal cross-reactivity between the first and second compositions.

Alternative preferred viral vectors for use in the first composition according to the invention include a variety of different viruses, genetically disabled so as to be non-replicating or replication-impaired. Such viruses include for example non-replicating adenoviruses such as E1 deletion mutants. Genetic disabling of viruses to produce non-replicating or replication-impaired vectors is well known.

Other suitable viral vectors for use in the first composition are vectors based on herpes virus and Venezuelan equine encephalitis virus (VEE). Suitable bacterial vectors for the first composition include recombinant BCG and recombinant Salmonella and Salmonella transformed with plasmid DNA (Darji A et al 1997 Cell 91: 765-775).

Alternative suitable non-viral vectors for use in the priming composition include lipid-tailed peptides known as lipopeptides, peptides fused to carrier proteins such as KLH either as fusion proteins or by chemical linkage, whole antigens with adjuvant, and other similar systems.

In one preferred embodiment of the invention, the source of T cell epitopes in the first composition is a nucleic acid, which may be DNA or RNA, in particular a recombinant DNA plasmid. The DNA or RNA may be packaged, for example in a lysosome, or it may be in free form.

In another preferred embodiment of the invention, the source of T cell epitopes in the first composition is a peptide, polypeptide, protein, polyprotein or particle comprising two or more CD4+ T cell epitopes, present in a recombinant string of CD4+ T cell epitopes or in a target antigen. Polyproteins include two or more proteins which may be the same, or preferably different, linked together. Preferably the epitopes in or encoded by the first or second composition are provided in a sequence which does not occur naturally as the expressed product of a gene in the parental organism from which the target antigen may be derived.

Preferably, the source of T cell epitopes in the second composition is a vaccinia virus vector such as MVA or NYVAC. Most preferred is the vaccinia strain modified virus ankara (MVA) or a strain derived therefrom (more detail on MVA is provided below). Alternatives to vaccinia vectors include avipox vectors such as fowl pox or canarypox vectors. Particularly suitable as an avipox vector is a strain of canarypox known as ALVAC (commercially available as Kanapox), and strains derived therefrom.

There is no reason why the first and second compositions should not be identical in that they may both contain the source of CD4+ T cell epitopes. A single formulation which can be used as a primer and as a booster will simplify administration.

Poxvirus Vectors

In the “second” composition used in the method of the present invention the source of the CD4+ (and optionally CD8+) epitopes is a non-replicating or replication impaired recombinant poxvirus vector.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

Replication of a virus is generally measured in two ways: 1) DNA synthesis and 2) viral titre. More precisely, the term “nonreplicating or replication-impaired” as used herein and as it applies to poxviruses means viruses which satisfy either or both of the following criteria:

1) exhibit a 1 log (10 fold) reduction in DNA synthesis compared to the Copenhagen strain of vaccinia virus in MRC-5 cells (a human cell line);

2) exhibit a 2 log reduction in viral titre in HELA cells (a human cell line) compared to the Copenhagen strain of vaccinia virus.

Examples of poxviruses which fall within this definition are MVA, NYVAC and avipox viruses, while a virus which falls outside the definition is the attenuated vaccinia strain M7.

Modified vaccinia virus Ankara (MVA) is a strain of vaccinia virus which does not replicate in most cell types, including normal human tissues. MVA was derived by serial passage >500 times in chick embryo fibroblasts (CEF) of material derived from a pox lesion on a horse in Ankara, Turkey (Mayr et al. Infection (1975) 33: 6-14.). It was shown to be replication-impaired yet able to induce protective immunity against veterinary poxvirus infections. MVA was used as a human vaccine in the final stages of the smallpox eradication campaign, being administered by intracutaneous, subcutaneous and intramuscular routes to >120,000 subjects in southern Germany. No significant side effects were recorded, despite the deliberate targeting of vaccination to high risk groups such as those with eczema (Mayr et al. Bakteriol B. (1978)167: 375- 90).

The safety of MVA reflects the avirulence of the virus in animal models, including irradiated mice and following intracranial administration to neonatal mice. The non-replication of MVA has been correlated with the production of proliferative white plaques on chick chorioallantoic membrane, abortive infection of non-avian cells, and the presence of six genomic deletions totalling approximately 30 kb. The avirulence of MVA has been ascribed partially to deletions affecting host range genes K1 L and C7L, although limited viral replication still occurs on human TK-143 cells and African Green Monkey CV-1 cells. Restoration of the K1 L gene only partially restores MVA host range. The host range restriction appears to occur during viral particle maturation, with only immature virions being observed in human HeLa cells on electron microscopy (Sutter et al. 1992). The late block in viral replication does not prevent efficient expression of recombinant genes in MVA.

Poxviruses have evolved strategies for evasion of the host immune response that include the production of secreted proteins that function as soluble receptors for tumour necrosis factor, IL-I p, interferon (IFN)-oc/andIFN-y, which normally have sequence similarity to the extracellular domain of cellular cytokine receptors (such as chemokine rcecptors).

These viral receptors generally inhibit or subvert an appropriate host immune response, and their presence is associated with increased pathogenicity. The Il-I p receptor is an exception: its presence diminishes the host febrile response and enhances host survival in the face of infection. MVA lacks functional cytokine receptors for interferon y, interferon ap, Tumour Necrosis Factor and CC chemokines, but it does possess the potentially beneficial IL-1 receptor. MVA is the only known strain of vaccinia to possess this cytokine receptor profile, which theoretically renders it safer and more immunogenicthan other poxviruses. Another replication impaired and safe strain of vaccinia known as NYVAC is fully described in Tartaglia et al.(Virology 1992, 188: 217-232).

Poxvirus genomes can carry a large amount of heterologous genetic information. Other requirements for viral vectors for use in vaccines include good immunogenicity and safety. In one embodiment the poxvirus vector may be a fowlpox vector, or derivative thereof.

It will be evident that vaccinia virus strains derived from MVA, or independently developed strains having the features of MVA which make MVA particularly suitable for use in a vaccine, will also be suitable for use in the invention.

MVA containing an inserted string of epitopes (as described in the Example 2) has been previously described in WO 98/56919.

Vaccination Strategies

The present inventors have shown that replication-defective pox viruses are capable of inducing effector CD4 T cells (optionally with CD8+ T cells) when used in heterologous prime-boost regimes.

Surprisingly, strong responses were obtained using a heterologous immunisation regime with the first and second compositions in either order. A slightly stronger was response was observed when the second composition was administered after the first, rather than the reverse order. Preferably, therefore, the method of the second embodiment of the invention comprises administering at least one dose of the first composition, followed by at least one dose of the second composition.

The method of the second embodiment of the invention may comprise administering a plurality of doses of the first composition, followed by at least one dose of the second composition.

Alternatively, the method of the second embodiment of the invention may comprise administering a plurality of doses of the first copmposition, followed by at least one dose of the second composition.

A particularly effective immunisation protocol has been found to be the administration of three sequential doses of the first composition, followed by one dose of the second composition.

The timing of the individual doses will depend on the individual (see “Administration” below) but will commonly be in the region of one to six weeks apart, usually about three weeks apart.

Target Antigens

The target antigen may be characteristic of the target disease. If the disease is an infectious disease, caused by an infectious pathogen, then the target antigen may be derivable from the infectious pathogen.

The target antigen may be an antigen which is recognised by the immune system after infection with the disease. Alternatively the antigen may be normally “invisible” to the immune system such that the method induces a non-physiological T cell response. This may be helpful in diseases where the immune response triggered by the disease is not effective (for example does not succeed in clearing the infection) since it may open up another line of attack.

The antigen may be a tumor antigen, for example MAGE-b 1, MAGE-3 or NY-ESO.

The antigen may be an autoantigen, for example tyrosinase.

In a preferred embodiment of the invention, the antigen is derivable from M. tuberculosis. For example, the antigen may be ESAT6 or MPT63.

In another preferred embodiment of the invention, the antigen is derivable from the malaria-associated pathogen P. Falciparum.

The compositions of the present invention may comprise T cell epitopes from more than one antigen (see above under “epitopes”). For example, the composition may comprise one or more T cell epitopes from two or more antigens associated with the same disease. The two or more antigens may be derivable from the same pathogenic organism.

Alternatively, the composition may comprise epitopes from a variety of sources. For example, the ME-TRAP insert described in the examples comprises T cell epitopes from P. falciparum, tetanus toxoid, M. tuberculosis and M. bovis.

Target Diseases

The method of the present invention will be useful in the prevention of any disease for which the presence of CD4+ T cells (in particular of the T_{H}1 type) is likely to contribute to protective immunity.

In particular, the method of the present invention will be useful in the prevention of diseases such as tuberculosis, HIV, malaria. H. pylori, influenza, hepatitis (e.g., HBV, HCV, CMV, herpes virus-induced diseases (e.g., HSV), Epstein Barr Virus (EBV), respiratory syncytial virus (RSV) and other viral infections, leprosy, non-malarial protozoan parasites such as toxoplasma, and various malignancies (e.g., prostrate, breast, lung, colorectal, melanoma, renal cancers and/or tumors; virally induced tumors).

The method of the present invention will be useful in the treatment of any disease for which the presence of CD4+ T cells (in particular of the T_{H}1 type) is likely to be therapeutic. In particular the method of the present invention is likely to be useful in therapeutic vaccination against tuberculosis, persistent viral infections such as HIV and chronic hepatitis B and C and many malignancies.

The method of the present invention is particularly useful in vaccination strategies to protect against tuberculosis.

The pox virus vector described herein may be particularly useful for boosting CD4 T cell responses in HIV-positive individuals.

The compositions described herein may be employed as therapeutic or prophylactic vaccines. Whether prophylactic or therapeutic immunisation is the more appropriate will usually depend upon the nature of the disease. For example, it is anticipated that cancer will be immunised against therapeutically rather than before it has been diagnosed, while anti-malaria vaccines will preferably, though not necessarily be used as a prophylactic.

Kits

The first and second compositions used in the method of the invention may conveniently be provided in the form of a “combined preparation” or kit. The first and second compositions may be packaged together or individually for separate sale. The first and second compositions may be used simultaneously, separately or sequentially for inducing a CD4+ T cell response against a target antigen.

The kit may comprise other components for mixing with one or both of the compositions before administration (such as diluents, carriers, adjuvants etc.—see below).

The kit may also comprise written instructions concerning the vaccination protocol.

Pharmaceutical Compositions/Vaccines

The present invention also relates to a product comprising the first and second compositions as defined above, and a medicament for boosting a primed CD4+ T cell response. The product/medicament may be in the form of a pharmaceutical composition.

The pharmaceutical composition may also comprise, for example, a pharmaceutically acceptable carrier, diluent, excipient or adjuvant. The choice of pharmaceutical carrier, excipient or diluent can be selected with regard to the intended route of administration and standard pharmaceutical practice.

In particular, a composition comprising a DNA plasmid vector may comprise granulocyte macrophage-colony stimulating factor (GM-CSF), or a plasmid encoding it, to act as an adjuvant; beneficial effects are seen using GM-CSF in polypeptide form. Adjuvants such as QS21 or SBAS2 (Stoute J A et al. 1997 N Engl J Medicine 226: 86-91) may be used with proteins, peptides or nucleic acids to enhance the induction of T cell responses.

In the pharmaceutical compositions of the present invention, the composition may also be admixed with any suitable binder(s), lubricant(s), suspending agent(s), coating agent(s), or solubilising agent(s).

The pharmaceutical composition could be for veterinary (i.e. animal) usage or for human usage.

Administration

In general, a therapeutically effective daily oral or intravenous dose of the compositions of the present invention is likely to range from 0.01 to 50 mg/kg body weight of the subject to be treated, preferably 0.1 to 20 mg/kg. The compositions of the present invention may also be administered by intravenous infusion, at a dose which is likely to range from 0.001-10 mg/kg/hr.

Tablets or capsules of the agents may be administered singly or two or more at a time, as appropriate. It is also possible to administer the compositions of the present invention in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

For oral, parenteral, buccal and sublingual administration to subjects (such as patients), the daily dosage level of the agents of the present invention may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

In some applications, generally, in humans, oral administration of the agents of the present invention is the preferred route, being the most convenient and can in some cases avoid disadvantages associated with other routes of administration—such as those associated with intracavernosal (i.c.) administration. In circumstances where the recipient suffers from a swallowing disorder or from impairment of drug absorption after oral administration, the drug may be administered parenterally, e.g. sublingually or buccally.

For veterinary use, the composition of the present invention is typically administered as a suitably acceptable formulation in accordance with normal veterinary practice and the veterinary surgeon will determine the dosing regimen and route of administration which will be most appropriate for a particular animal. However, as with human treatment, it may be possible to administer the composition alone for veterinary treatments.

Example Formulations and Immunization Protocols

Formulation 1

| | |
|---|---|
| Priming Composition: | DNA plasmid 1 mg/ml in PBS |
| Boosting Composition: | Recombinant MVA, 10⁸ ffu in PBS |

Protocol: Administer two doses of 1 mg of priming composition, i.m., at 0 and 3 weeks followed by two doses of booster intradermally at 6 and 9 weeks.

Formulation 2

| | |
|---|---|
| Priming Composition: | Ty-VLP 500 μg in PBS |
| Boosting Composition: | MVA, 10⁸ ffu in PBS |

Protocol: Administer two doses of priming composition, i.m., at 0 and 3 weeks, then 2 doses of booster at 6 and 9 weeks. For tumor treatment, MVA is given i.v. as one of most effective routes.

Formulation 3

| | |
|---|---|
| Priming Composition: | Protein 500 μg + adjuvant (QS-21) |
| Boosting Composition: | Recombinant MVA, 10⁸ ffu in PBS |

Protocol: Administer two doses of priming composition at 0 and 3 weeks and 2 doses of booster i.d. at 6 and 9 weeks.

Formulation 4

| | |
|---|---|
| Priming Composition: | Adenovirus vector, 10⁹ pfu in PBS |
| Boosting Composition: | Recombinant MVA, 10⁸ ffu in PBS |

Protocol: Administer one or two doses of priming composition intradermally at 0 and 3 weeks and two doses of booster i.d. at 6 and 9 weeks.

The above doses and protocols may be varied to optimise protection. Doses may be given between for example, 1 to 8 weeks apart rather than 2 weeks apart.

The invention will now be further described in the examples which follow.

### EXAMPLES

### Example 1

### Materials and Methods

Generation of the Epitope Strings

The malaria epitope string was made up of a series of cassettes each encoding three epitopes as shown in Table 1, with restriction enzyme sites at each end of the cassette. Each cassette was constructed from four synthetic oligonucleotides which were annealed together, ligated into a cloning vector and then sequenced to check that no errors had been introduced. Individual cassettes were then joined together as required. The BamHI site at the 3′ end of cassette C was fused to the BglII site at the 5′ end of cassette A, destroying both restriction enzyme sites and encoding a two amino acid spacer (GS) between the two cassettes. Cassettes B, D and H were then joined to the string in the same manner. A longer string containing CABDHFE was also constructed in the same way.

TABLE 1

CTL Epitopes of the Malaria (M) String

| | | Amino acid | | | HLA |
|---|---|---|---|---|---|
| Cassette | Epitope | Sequence | DNA sequence | Type | restriction |
| A | Ls8 | KPNDKSLY | AAGCCGAACGACAAGTCCTTGTAT | CTL | B35 |
| | | SEQ ID NO.:2 | SEQ ID NO.:1 | | |
| | Cp26 | KPKDELDY | AAACCTAAGGACGAATTGGACTAC | CTL | B35 |
| | | SEQ ID NO.:4 | SEQ ID NO.:3 | | |
| | Ls6 | KPIVQYDNF | AAGCCAATCGTTCAATACGACAACTTC | CTL | B53 |
| | | SEQ ID NO.:6 | SEQ ID NO.:5 | | |
| B | Tr42/43 | ASKNKEKALII | GCCTCCAAGAACAAGGAAAAGGCTTTG | CTL | B8 |
| | | SEQ ID NO.:8 | ATCATC SEQ ID NO.:7 | | |
| | Tr39 | GIAGGLALL | GGTATCGCTGGTGGTTTGGCCTTGTTG | CTL | A2.1 |
| | | SEQ ID NO.:10 | SEQ ID NO.:9 | | |
| | Cp6 | MNPNDPNRNV | ATGAACCCTAATGACCCAAACAGAAAC | CTL | B7 |
| | | SEQ ID NO.:12 | GTC SEQ ID NO.:11 | | |
| C | St8 | MINAYLDKL | ATGATCAACGCCTACTTGGACAAGTTG | CTL | A2.2 |
| | | SEQ ID NO.:14 | SEQ ID NO.:13 | | |
| | Ls50 | ISKYEDEI | ATCTCCAAGTACGAAGACGAAATC | CTL | B17 |
| | | SEQ ID NO.:16 | SEQ ID NO.:15 | | |
| | Pb9 | SYIPSAEKI | TCCTACATCCCATCTGCCGAAAAGATC | CTL | mouse H2- |
| | | SEQ ID NO.:18 | SEQ ID NO.:17 | | K^{d} |
| D | Tr26 | HLGNVKYLV | CACTTGGGTAACGTTAAGTACTTGGTT | CTL | A2.1 |
| | | SEQ ID NO.:20 | SEQ ID NO.:19 | | |
| | Ls53 | KSLYDEHI | AAGTCTTTGTACGATGAACACATC | CTL | B58 |
| | | SEQ ID NO.:22 | SEQ ID NO.:21 | | |
| | Tr29 | LLMDCSGSI | TTATTGATGGACTGTTCTGGTTCTATT | CTL | A2.2 |
| | | SEQ ID NO.:24 | SEQ ID NO.:23 | | |
| E | NANP | NANPNANPNANP | AACGCTAATCCAAACGCAAATCCGAAC | B cell | |
| | | NANP | GCCAATCCTAACGCGAATCCC | | |
| | | SEQ ID NO.:26 | SEQ ID NO.:25 | | |
| | TRAP | DEWSPCSVTCGK | GACGAATGGTCTCCATGTTCTGTCACTT | Heparin | |
| | AM | GTRSRKRE | GTGGTAAGGGTACTCGCTCTAGAAAGA | binding | |
| | | SEQ ID NO.:28 | GAGAA SEQ ID NO.:27 | motif | |
| F | Cp39 | YLNKIQNSL | TACTTGAACAAAATTCAAAACTCTTTG | CTL | A2.1 |
| | | SEQ ID NO.:30 | SEQ ID NO.:29 | | |
| | La72 | MEKLKELEK | ATGGAAAAGTTGAAAGAATTGGAAAAG | CTL | B8 |
| | | SEQ ID NO.:32 | SEQ ID NO.:31 | | |
| | ex23 | ATSVLAGL | GCTACTTCTGTCTTGGCTGGTTTG | CTL | B58 |
| | | SEQ ID NO.:34 | SEQ ID NO.:33 | | |
| H | CSP | DPNANPNVDPNA | GACCCAAACGCTAACCCAAACGTTGAC | T helper | Universal |
| | | NPNV | CCAAACGCCAACCCAAACGTC | | |
| | | SEQ ID NO.:36 | SEQ ID NO: 35 | | |
| | BCG | QVHFQPLPPAVV | CAAGTTCACTTCCAACCATTGCCTCCGG | T helper | epitopes |
| | | KL | CCGTTGTCAAGTTG | | |
| | | SEQ ID NO:38 | SEQ ID NO.:37 | | |
| | TT | QFIKANSKFIGIT | CAATTCATCAAGGCCAACTCTAAGTTCA | T helper | |
| | | E | TCGGTATCACCGAA | | |
| | | SEQ D NO.:40 | SEQ ID NO.:39 | | |

Table 1

Sequences included in the malaria epitope string. Each cassette consists of the epitopes shown above, in the order shown, with no additional sequence between epitopes within a cassette. A BglII site was added at the 5′ end and a BamHI site at the 3′ end, such that between cassettes in an epitope string the BamHI/BglII junction encodes GS. All epitopes are from P. falciparum antigens except for pb9 (P. berghei), BCG (M. tuberculosis) and TT (Tetanus). The amino acid and DNA sequences shown in the table have SEQ ID NOS. 1 to 40 in the order in which they appear.

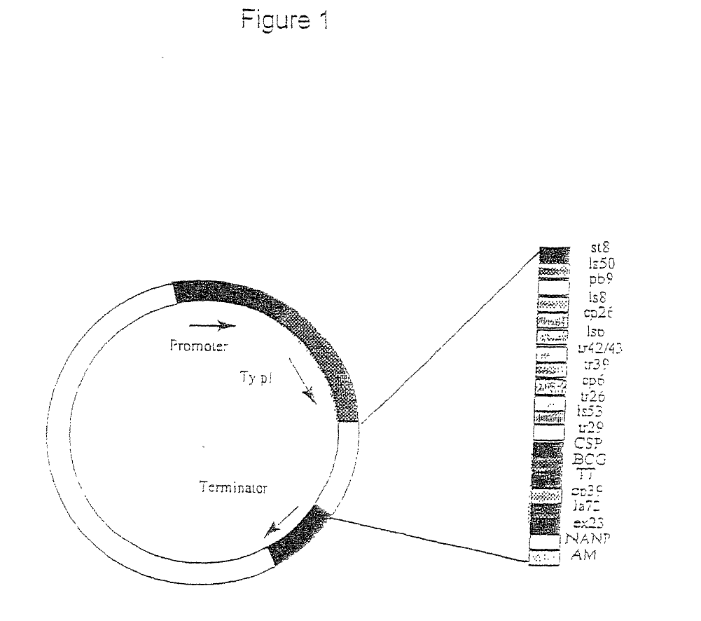

FIG. 1 shows the construct used to express Ty-VLP with the malaria epitope cassette CABDHFE. CTL epitopes are from P. falciparum STARP (sporozoite threonine- and asparagine-rich protein) (st), LSA-1 (liver stage antigen 1) (1s), CSP (circumsporozoite protein) (cp), TRAP (thrombospondin-related adhesive protein) (tr), LSA-3 (liver stage antigen 3) (1a) and Exp-1 (exported protein 1) (ex). Helper epitopes are from the P. falciparum CS protein, the M. tuberculosis 38Kd antigen and Tetanus Toxoid. NANP is the antibody epitope from CS and AM is the adhesion motif from P. falciparum TRAP (Muller et al., EMBO-J., 12: 2881-9 (1993)). The length of the complete string is 229 amino acids as shown in the table 1 legend, with the amino acid sequence:

| | |
|---|---|
| MINAYLDKLISKYEDEISYIPSAEKIGSKPNDKSLYKPKDELDYKPIVQYDNFGSA | [SEQ ID NO: 41] |
| SKNKEKALIIGIAGGLALLMNPNDPNRNVGSHLGNVKYLVKSLYDEHILLMDCS | |
| GSIGSDPNANPNVDPNANPNVQVHFQPLPPAVVKLQFIKANSKFIGITEGSYLNKI | |
| QNSLMEKLKELEKATSVLAGLGSNANPNANPNANPNANPDEWSPCSVTCGKG | |
| TRSRKREGSGK. | |

The HIV epitope string was also synthesised by annealing oligonucleotides. Finally the HIV and malaria epitope strings were fused by joining the BamHI site at the 3′ end of the HIV epitopes to the BglII site at the 5′ end of cassettes CAB to form the HM string (Table 2).

TABLE 2

CTL Epitopes of the HIV/SIV Epitope String

| | | |
|---|---|---|
| Epitope | Restriction | Origin |

| | | | |
|---|---|---|---|
| YLKDQQLL | (SEQ ID NO.:42) | A24, B8 | HIV-1 gp41 |
| ERYLKDQQL | (SEQ ID NO.: 43) | B14 | HIV-1 gp41 |
| EITPIGLAP | (SEQ ID NO.: 44) | Mamu-B*01 | SIV env |
| PPIPVGEIY | (SEQ ID NO.: 45) | B35 | HIV-1 p24 |
| GEIYKRWII | (SEQ ID NO.: 46) | B8 | HIV-1 p24 |
| KRWIILGLNK | (SEQ ID NO.: 47) | B*2705 | HIV-1 p24 |
| IILGLNKIVR | (SEQ ID NO.: 48) | A33 | HIV-1 p24 |
| LGLNKIVRMY | (SEQ ID NO.: 49) | Bw62 | HIV-1 p24 |
| YNLTMKCR | (SEQ ID NO.: 50) | Mamu-A*02 | SIV env |
| RGPGRAFVTI | (SEQ ID NO.: 51) | A2, H-2Dd | HIV-1 gp120 |
| GRAFVTIGK | (SEQ ID NO.: 52) | B*2705 | HIV-1 gp120 |
| TPYDINQML | (SEQ ID NO.: 53) | B53 | HIV-2 gag |
| CTPYDINQM | (SEQ ID NO.: 54) | Mamu-A*01 | SIV gag |
| RPQVPLRMTY | (SEQ ID NO.: 55) | B51 | HIV-1 nef |
| QVPLRPMTYK | (SEQ ID NO.: 56) | A*0301, A11 | HIV-1 nef |
| VPLRPMTY | (SEQ ID NO.: 57) | B35 | HIV-1 nef |
| AVDLSHFLK | (SEQ ID NO.: 58) | A11 | HIV-1 nef |
| DLSHFLKEK | (SEQ ID NO.: 59) | A*0301 | HIV-1 nef |
| FLKEKGGL | (SEQ ID NO.: 60) | B8 | HIV-1 nef |
| ILKEPVHGV | (SEQ ID NO.: 61) | A*0201 | HIV-1 pol |
| ILKEPVHGVY | (SEQ ID NO.: 62) | Bw62 | HIV-1 pol |
| HPDIVIYQY | (SEQ ID NO.: 63) | B35 | HIV-1 pol |
| VIYQYMDDL | (SEQ ID NO.: 64) | A*0201 | HIV-1 pol |

Table 2

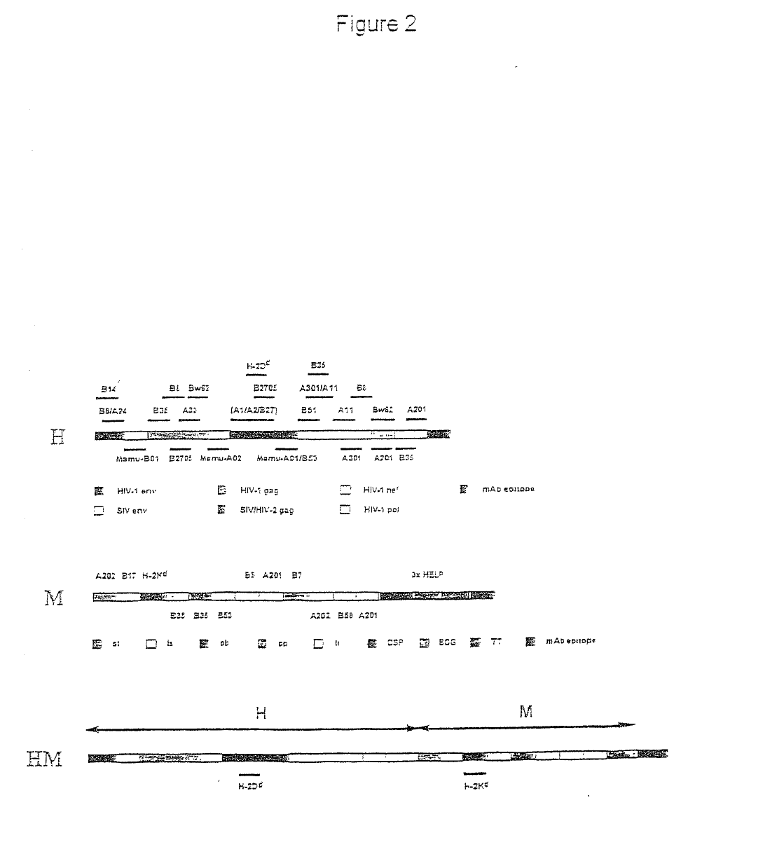

Sequences of epitopes from HIV or SIV contained in the H epitope string and assembled as shown in FIG. 2. The amino acids in the table have SEQ ID NOS: 42 to 64 in the order in which they appear.

FIG. 2 shows a schematic outline of the H, M and HM proteins. The bar patterns on the schematic representations of the polyepitope proteins indicate the origin of the sequences (see tables 1 and 2). The positions of individual epitopes and their MHC restrictions are depicted above and below the proteins. Pb is the only epitope derived from the protein of P. berghei. All other epitopes in the M protein originate from proteins of P. falciparum: cs—circumsporozoite protein, st—STARP, Is—LSA-1 and tr—TRAP. BCG—38 kDa protein of M. tuberculosis; TT—tetanus toxin.

For the anti-tumour vaccine an epitope string containing CTL epitopes was generated, similar to the malaria and HIV epitope string. In this tumour epitope string published murine CTL epitopes were fused together to create the tumour epitope string with the amino acid sequence: MLPYLGWLVF-AQHPNAELL-KHYLFRNL-SPSYVYHQF-IPNPLLGLD [SEQ ID NO: 65]. CTL epitopes shown here were fused together. The first amino acid methionine was introduced to initiate translation.

Ty Virus-Like Particles (Vlps)

The epitope string containing cassette CABDH was introduced into a yeast expression vector to make a C-terminal in-frame fusion to the TyA protein. When TyA or TyA fusion proteins are expressed in yeast from this vector, the protein spontaneously forms virus like particles which can be purified from the cytoplasm of the yeast by sucrose gradient centrifugation. Recombinant Ty-VLPs were prepared in this manner and dialysed against PBS to remove the sucrose before injection (c.f. Layton et al., Immunology, 87: 171-8 (1996)).

Adenoviruses

Replication-defective recombinant Adenovirus with a deletion of the E1 genes was used in this study (McGrory et al, Virology, 163: 614-7 (1988)). The Adenovirus expressed E. coli β-galactosidase under the control of a CMV IE promoter. For immunisations, 10⁷ pfu of virus were administered intradermally into the ear lobe.

Peptides

Peptides were purchased from Research Genetics (USA), dissolved at 10 mg/ml in DMSO (Sigma) and further diluted in PBS to 1 mg/ml. Peptides comprising CTL epitopes that were used in the experiments described herein are listed in table 3.

TABLE 3

Sequence of CTL Peptide Epitopes

| | | |
|---|---|---|
| sequence | Antigen | MHC restriction |

| | | | |
|---|---|---|---|
| LPYLGWLVF | (SEQ ID NO.:66) | P1A tumour antigen | L^{d} |
| SYIPSAEKI | (SEQ ID NO.:67) | P. berghei CSP | K^{d} |
| RGPGRAFVTI | (SEQ ID NO.:68) | HIV gag | D^{d} |
| TPHPARIGL | (SEQ ID NO.:69) | E. coli b-galactosidase | L^{d} |
| TYQRTRALV | (SEQ ID NO.:70) | Influenza A virus NP | K^{d} |
| SDYEGRLI | (SEQ ID NO.:71) | Influenza A virus NP | K^{k} |
| ASNENMETM | (SEQ ID NO.:72) | Influenza A virus NP | D^{b} |
| INVAFNRFL | (SEQ ID NO.:73) | P. falciparum TRAP | K^{b} |

The amino acid sequences in Table 3 have SEQ ID NOS: 66 to 73, in the order in which they appear in the Table.

Plasmid DNA Constructs

A number of different vectors were used for constructing DNA vaccines. Plasmid pTH contains the CMV IE promoter with intron A, followed by a polylinker to allow the introduction of antigen coding sequences and the bovine growth hormone transcription termination sequence. The plasmid carries the ampicillin resistance gene and is capable of replication in E. coli but not mammalian cells. This was used to make DNA vaccines expressing each of the following antigens: P. berghei TRAP, P. berghei CS, P. falciparum TRAP, P. falciparum LSA-1 (278 amino acids of the C terminus only), the epitope string containing cassettes CABDH and the HM epitope string (HIV epitopes followed by cassettes CAB). Plasmid pSG2 is similar to pTH except for the antibiotic resistance gene. In pSG2 the ampicillin resistance gene of pTH has been replaced by a kanamycin resistance gene. pSG2 was used to to make DNA vaccines expressing the following antigens: P. berghei PbCSP, a mouse tumour epitope string, the epitope string containing cassettes CABDH and the HM epitope string. Plasmid V1J-NP expresses influenza nucleoprotein under the control of a CMV IE promoter. Plasmids CMV-TRAP and CMV-LSA-1 are similar to pTH.TRAP and pTH. LSA-1 but do not contain intron A of the CMV promoter. Plasmids RSV.TRAP and RSV.LSA-1 contain the RSV promoter, SV40 transcription termination sequence and are tetracycline resistant. For induction of β-galactosidase-specific CTL plasmid pcDNA3/His/LacZ (Invitrogen) was used. All DNA vaccines were prepared from E. coli strain DH5α using Qiagen plasmid purification columns.

Generation of Recombinant Vaccinia Viruses

Recombinant MVAs were made by first cloning the antigen sequence into a shuttle vector with a viral promoter such as the plasmid pSC11 (Chakrabarti et al., Mol. Cell. Biol., 5: 3403-9 (1985); Morrison et al., Virology, 171: 179-88 (1989)). P. berghei CS and P. falciparum TRAP, influenza nucleoprotein and the HM and mouse tumour epitope polyepitope string were each expressed using the P7.5 promoter (Mackett et al., J. Virol., 49: 857-864 (1984)), and P. berghei TRAP was expressed using the strong synthetic promoter (SSP; Carroll et al., Biotechnology, 19: 352-4 (1995)). The shuttle vectors, pSC11 or pMCO3 were then used to transform cells infected with wild-type MVA so that viral sequences flanking the promoter, antigen coding sequence and marker gene could recombine with the MVA and produce recombinants. Recombinant viruses express the marker gene (β glucuronidase or β galactosidase) allowing identification of plaques containing recombinant virus. Recombinants were repeatedly plaque purified before use in immunisations. The recombinant NYVAC-PbCSP vaccinia was previously described (Lanar et al., Infection and Immunity, 64: 1666-71 (1996)). The wild type or Western Reserve (WR) strain of recombinant vaccinia encoding PbCSP was described previously (Satchidanandam et al., Mol. Biochem. Parasitol., 48: 89-99 (1991)).

Cells and Culture Medium

Murine cells and Epstein-Barr virus transformed chimpanzee and macaque B cells (B CL) were cultured in RPMI supplemented with 10% heat inactivated fetal calf serum (FCS). Splenocytes were restimulated with the peptides indicated (final concentration 1 μg/ml) in MEM medium with 10% FCS, 2 mM glutamine, 50 U/ml penicillin, 50 μM 2-mercaptoethanol and 10 mM Hepes pH7.2 (Gibco, UK).

Animals

Mice of the strains indicated, 6-8 weeks old were purchased from Harlan Olac (Shaws Farm, Blackthorn, UK). Chimpanzees H1 and H2 were studied at the Biomedical Primate Research Centre at Rijswick, The Netherlands. Macaques were studied at the University of Oxford.

Immunisations

Plasmid DNA immunisations of mice were performed by intramuscular immunisation of the DNA into the musculus tibialis under anaesthesia. Mouse muscle was sometimes pre-treated with 50 μl of 1 mM cardiotoxin (Latoxan, France) 5-9 days prior to immunisation as described by Davis et al (J. Virol, Human Molecular Genetics, 2:1847-51 (1993)), but the presence or absence of such pre-treatment was not found to have any significant effect on immunogenicity or protective efficacy. MVA immunisation of mice was performed by either intramuscular (i.m.), intravenous (into the lateral tail vein) (i.v.), intradermal (i.d.), intraperitoneal (i.p.) or subcutaneous (s.c.) immunisation. Plasmid DNA and MVA immunisation of the chimpanzees H1 and H2 was performed under anaesthesia by intramuscular immunisation of leg muscles. For these chimpanzee immunisations the plasmid DNA was co-administered with 15 micrograms of human GM-CSF as an adjuvant. Recombinant MVA administration to the chimpanzees was by intramuscular immunisation under veterinary supervision. Recombinant human GM-CSF was purchased from Sandoz (Camberley, UK). For plasmid DNA immunisations using a gene gun, DNA was precipitated onto gold particles. For intradermal delivery, two different types of gene guns were used, the Acell and the Oxford Bioscience device (PowderJect Pharmaceuticals, Oxford, UK).

Elispot Assays

CD8+ T cells were quantified in the spleens of immunised mice without in vitro restimulation using the peptide epitopes indicated and the ELISPOT assay as described by Miyahara et al (J Immunol Methods,18: 45-54 (1993)). Briefly, 96-well nitrocellulose plates (Miliscreen MAHA, Millipore, Bedford UK) were coated with 15 μg/ml of the anti-mouse interferon-γ monoclonal antibody R4 (EACC) in 50 μl of phosphate-buffered saline (PBS). After overnight incubation at 4° C. the wells were washed once with PBS and blocked for 1 hour at room temperature with 100 μl RPMI with 10% FCS. Splenocytes from immunised mice were resuspended to 1×10⁷ cells/ml and placed in duplicate in the antibody coated wells and serially diluted. Peptide was added to each well to a final concentration of 1 μg/ml. Additional wells without peptide were used as a control for peptide-dependence of interferon-γ secretion. After incubation at 37° C. in 5% CO₂ for 12-18 hours the plates were washed 6 times with PBS and water. The wells were then incubated for 3 hours at room temperature with a solution of 1 μg/ml of biotinylated anti-mouse interferon-γ monoclonal antibody XMG1.2 (Pharmingen, Calif., USA) in PBS. After further washes with PBS, 50 μl of a 1 μg/ml solution of streptavidin-alkaline-phosphatase polymer (Sigma) was added for 2 hours at room temperature. The spots were developed by adding 50 μl of an alkaline phosphatase conjugate substrate solution (Biorad, Hercules, Calif., USA). After the appearance of spots the reaction was stopped by washing with water. The number of spots was determined with the aid of a stereomicroscope.

ELISPOT assays on the chimpanzee peripheral blood lymphocytes were performed using a very similar method employing the assay and reagents developed to detect human CD8 T cells (Mabtech, Stockholm).

CTL Assays

CTL assays were performed using chromium labelled target cells as indicated and cultured mouse spleen cells as effector cells as described by Allsopp et al., European Journal of Immunology, 26:1951-1959 (1996)). CTL assays using chimpanzee or macaque cells were performed as described for the detection of human CTL by Hill et al. (Nature 352: 595-600 (1991)) using EBV-transformed autologous chimpanzee chimpanzee or macaque B cell lines as target cells.

P. Berghei Challenge

Mice were challenged with 2000 (BALB/c) or 200 (C57BL/6) sporozoites of the P. berghei ANKA strain in 200 ml RPMI by intravenous inoculation as described (Lanar et al., Infection and Immunity, 64:1666-71 (1996)). These sporozoites were dissected from the salivary glands of Anopheles stephensi mosquitoes maintained at 18° C. for 20-25 days after feeding on infected mice. Blood-stage malaria infection, indicating a failure of the immunisation, was detected by observing the appearance of ring forms of P. berghei in Giemsa-stained blood smears taken at 5-12 days post-challenge.

P. Falciparum Challenge

The chimpanzees were challenged with 20,000 P. falciparum sporozoites of the NF54 strain dissected from the salivary glands of Anopheles gambiae mosquitoes, by intravenous inoculation under anaesthesia. Blood samples from these chimpanzees were examined daily from day 5 after challenge by microscopy and parasite culture, in order to detect the appearance of low levels of P. falciparum parasites in the peripheral blood.

P815 Tumour Challenges

Mice were challenged with 1×10⁵ P815 cells in 200 μl of PBS by intravenous inoculation. Animals were monitored for survival.

Influenza Virus Challenges

Mice were challenged with 100 haemagglutinating units (HA) of influenza virus A/PR/8/34 by intranasal inoculation. Following challenge the animals were weighed daily and monitored for survival.

Determining Peptide Specific CTL Using Tetramers

Tetrameric complexes consisting of Mamu-A*01-heavy chain and β₂-microglobulin were made as described by Ogg et al., Science, 279: 2103-6 (1998)). DNA coding for the leaderless extracellular portion of the Mamu-A*01 MHC class I heavy chain was PCR-amplified from cDNA using 5′ primer MamuNdeI: 5′-CCT GAC TCA GAC CAT ATG GGC TCT CAC TCC ATG [SEQ ID NO: 74] and 3′ primer: 5′-GTG ATA AGC TTA ACG ATG ATT CCA CAC CAT TTT CTG TGC ATC CAG AAT ATG ATG CAG GGA TCC CTC CCA TCT CAG GGT GAG GGG C [SEQ ID NO: 75]. The former primer contained a NdeI restriction site, the latter included a HindIII site and encoded for the bioinylation enzyme BirA substrate peptide. PCR products were digested with NdeI and HindIII and ligated into the same sites of the polylinker of bacterial expression vector pGMT7. The rhesus monkey gene encoding a leaderless β₂-microglobulin was PCR amplifed from a cDNA clone using primers B2MBACK: 5′-TCA GAC CAT ATG TCT CGC TCC GTG GCC [SEQ ID NO: 76] and B2MFOR: 5′-TCA GAC AAG CTT TTA CAT GTC TCG ATC CCA C [SEQ ID NO: 77] and likewise cloned into the NdeI and HindIII sites of pGMT7. Both chains were expressed in E. coil strain BL-21, purified from inclusion bodies, refolded in the presence of peptide CTPYDINQM [SEQ ID NO: 54], biotinylated using the BirA enzyme (Avidity) and purified with FPLC and monoQ ion exchange columns. The amount of biotinylated refolded MHC-peptide complexes was estimated in an ELISA assay, whereby monomeric complexes were first captured by conformation sensitive monoclonal antibody W6/32 and detected by alkaline phosphatase (AP)—conjugated streptavidin (Sigma) followed by colorimetric substrate for AP. The formation of tetrameric complexes was induced by addition of phycoerythrin (PE)—conjugated streptavidin (ExtrAvidin; Sigma) to the refolded biotinylated monomers at a molar ratio of MHC-peptide : PE-streptavidin of 4:1. The complexes were stored in the dark at 4° C. These tetramers were used to analyse the frequency of Mamu-A*01/gag-specific CD8+ T cells in peripherial blood lymphocytes (PBL) of immunised macaques.

### Example 2

Immunogenicity Studies in Mice

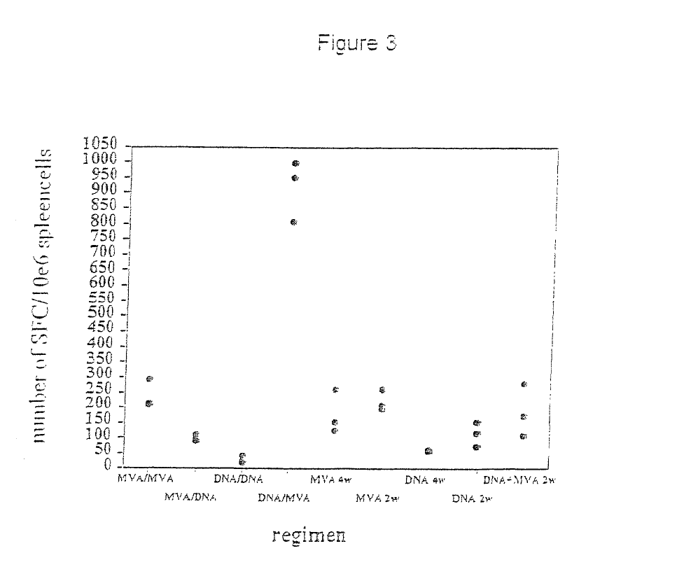

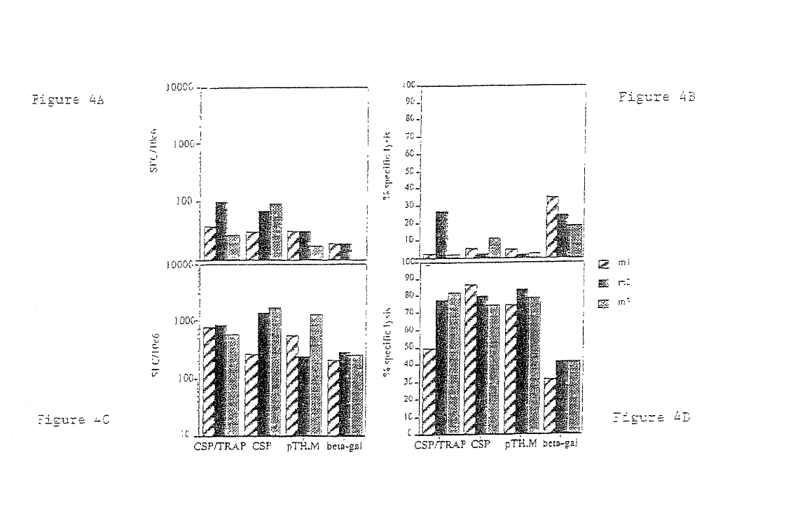

Previous studies of the induction of CTL against epitopes in the circumsporozoite (CS) protein of Plasmodium berghei and Plasmodium yoelii have shown variable levels of CTL induction with different delivery systems. Partial protection has been reported with plasmid DNA (Sedegah et al., Proc. Natl. Acad. Sci USA, 91: 9866-70 (1994)), influenza virus boosted by replicating vaccinia virus (Li et al., Proc. Natl. Acad. Sci. USA, 90:5214-8 (1991)), adenovirus (Rodrigues et al., Journal of Immunology, 158: 1268-74 (1997)) and particle delivery systems (Schodel et al., J. Exp. Med.,180: 1037-46 (1994)). Immunisation of mice intramuscularly with 50 micrograms of a plasmid encoding the CS protein produced moderate levels of CD8+ cells and CTL activity in the spleens of these mice after a single injection (FIGS. 3, 4A-4D).

For comparison groups of BALB/c mice (n=5) were injected intravenously with 10⁶ ffu/pfu of recombinant vaccinia viruses of different strains (WR, NYVAC and MVA) all expressing P. berghei CSP. The frequencies of peptide-specific CD8+ T cells were measured 10 days later in an ELISPOT assay. MVA.PbCSP induced 181+/−48, NYVAC 221+/−27 and WR 94+/−19 (mean +/− standard deviation) peptide-specific CD8+ T cells per million splenocytes. These results show that surprisingly replication-impaired vaccinia viruses are superior to replicating strains in priming a CD8+ T cell response. We then attempted to boost these moderate CD8+ T cell responses induced by priming with either plasmid DNA or MVA using homologous or heterologous vectors. A low level of CD8+ T cells was observed after two immunisations with CS recombinant DNA vaccine alone, the recombinant MVA vaccine alone or the recombinant MVA followed by recombinant DNA (FIG. 3). A very much higher level of CD8+ T cells was observed by boosting the DNA-primed immune response with recombinant MVA. In a second experiment using ten mice per group the enhanced immunogenicity of the DNA/MVA sequence was confirmed: DNA/MVA 856+/−201; MVA/DNA 168+/−72; MVA/MVA 345+/−90; DNA/DNA 92+/−46. Therefore the sequence of a first immunisation with a recombinant plasmid encoding the CS protein followed by a second immunisation with the recombinant MVA virus yielded the highest levels of CD8+ T lymphocyte response after immunisation.

FIG. 3 shows malaria CD8 T cell ELISPOT data following different immunisation regimes. Results are shown as the number of peptide-specific T cells per million splenocytes. Mice were immunised either with the PbCSP-plasmid DNA or the PbCSP-MVA virus or combinations of the two as shown on the X axis, at two week intervals and the number of splenocytes specific for the pb9 malaria epitope assayed two weeks after the last immunisation. Each point represents the number of spot-forming cells (SFCs) measured in an individual mouse. The highest level of CD8+ T cells was induced by priming with the plasmid DNA and boosting with the recombinant MVA virus. This was more immunogenic than the reverse order of immunisation (MVA/DNA), two DNA immunisations (DNA/DNA) or two MVA immunisations (MVA/MVA). It was also more immunogenic than the DNA and MVA immunisations given simultaneously (DNA+MVA 2w), than one DNA immunisation (DNA 4w) or one MVA immunisation given at the earlier or later time point (MVA 2w and MVA 4w).

FIGS. 4A-4D shows that malaria CD8 T cell ELISPOT and CTL levels are substantially boosted by a recombinant MVA immunisation following priming with a plasmid DNA encoding the same antigen. A AND C. CD8+ T cell responses were measured in BALB/c mice using the g-interferon ELISPOT assay on fresh splenocytes incubated for 18 h with the K^{d} restricted peptide SYIPSAEKI [SEQ ID NO: 67] from P. berghei CSP and the L^{d} restricted peptide TPHPARIGL [SEQ ID NO: 69] from E. coli β-galactosidase. Note that the ELISPOT counts are presented on a logarithmic scale. B and D. Splenocytes from the same mice were also assayed in conventional ⁵¹Cr-release assays at an effector: target ration of 100:1 after 6 days of in vitro restimulation with the same peptides (1 μg/ml).

The mice were immunised with plasmid DNA expressing either P. berghei CSP and TRAP, PbCSP alone, the malaria epitope cassette including the P. berghei CTL epitope (labelled pTH.M), or β-galactosidase. ELISPOT and CTL levels measured in mice 23 days after one DNA immunisation are shown in A and B respectively. The same assays were performed with animals that received additionally 1×10⁷ ffu of recombinant MVA expressing the same antigen(s) two weeks after the primary immunisation. The ELISPOT and CTL levels in these animals are shown in C and D respectively. Each bar represents data from an individual animal.

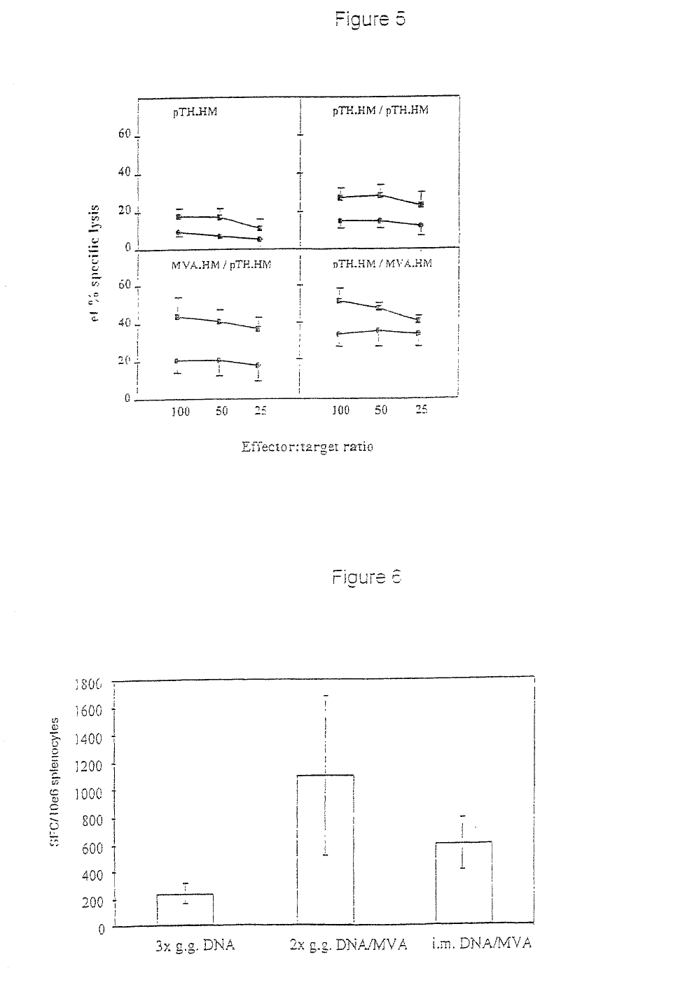

Studies were also undertaken of the immunogenicity of the epitope string IBM comprising both HIV and malaria epitopes in tandem. Using this epitope string again the highest levels of CD8+ T cells and CTL were generated in the spleen when using an immunisation with DNA vaccine followed by an immunisation with a recombinant MVA vaccine (Table 4, FIG. 5).

TABLE 4
Immunogenicity of Various DNA/MVA Combinations as Determined by
Elispot Assays

| Immunisation 1 | Immunisation 2 | HIV epitope | Malaria epitope |
|---|---|---|---|
| DNA-HM | DNA-HM | 56 ± 26 | 4 ± 4 |
| MVA-HM | MVA-HM | 786 ± 334 | 238 ± 106 |
| MVA-HM | DNA-HM | 306 ± 78 | 58 ± 18 |
| DNA-HM | MVA-HM | 1000 ± 487 | 748 ± 446 |
| None | DNA-HM | 70 ± 60 | 100 ± 10 |
| None | MVA-HM | 422 ± 128 | 212 ± 94 |

Table 4 shows the results of ELISPOT assays performed to measure the levels of specific CD8+ T cells to HIV and malaria epitopes following different immunisation regimes of plasmid DNA and MVA as indicated. The numbers are spot-forming cells per million splenocytes. The HM epitope string is illustrated in FIG. 2. BALB/c mice were used in all cases. The malaria epitope was pb9 as in FIGS. 2 and 3. The HIV epitope was RGPGRAFVTI [SEQ ID NO: 51]. The immunisation doses were 50 μg of plasmid DNA or 10⁷ focus-forming units (ffu) of recombinant MVA. All immunisations were intramuscular. The interval between immunisations 1 and 2 was from 14-21 days in all cases.

FIG. 5 shows the CTL responses induced in BALB/c mice to malaria and HIV epitopes by various immunisation regimes employing plasmid DNA and recombinant MVA. Mice were immunised intramuscularly as described in the legend to table 3 and in methods. High levels of CTL (>30% specific lysis at effector/target ration of 25:1) were observed to both the malaria and HIV epitopes only after priming with plasmid DNA and boosting with the recombinant MVA. The antigen used in this experiment is the HIV-malaria epitope string. The recombinant MVA is denoted MVA.HM and the plasmid DNA expressing this epitope string is denoted pTH.HM. Levels of specific lysis at various effector to target ratios are shown. These were determined after 5 days in vitro restimulation of splenocytes with the two peptides pb9 and RGPGRAFVTI [SEQ ID NO: 51].

Comparison of numerous delivery systems for the induction of CTL was reported by Allsopp et al., European Journal of Immunology, 26:1951-1959 (1996)). Recombinant Ty-virus like particles (Ty-VLPs) and lipid-tailed malaria peptides both gave good CTL induction but Ty-VLPs were better in that they required only a single immunising dose for good CTL induction. However, as shown here even two doses of Ty particles fail to induce significant protection against sporozoite challenge (Table 7, line 1). Immunisation with a recombinant modified vaccinia Ankara virus encoding the circumsporozoite protein of P. berghei also generates good levels of CTL. However, a much higher level of CD8+ T cell response is achieved by a first immunisation with the Ty-VLP followed by a second immunisation with the MVA CS vaccine (Table 5).

TABLE 5
Immunogenicity of Various Ty-VLP/MVA Combinations as Determined
by ELISPOT and CTL Assays

| Immunisation 1 | Immunisation 2 | ELISPOT No | % Specific Lysis |
|---|---|---|---|
| Ty-CABDH | Ty-CABDH | 75 | 15 |
| MVA.PbCSP | MVA.PbCSP | 38 | 35 |
| Ty-CABDH | MVA.PbCSP | 225 | 42 |
| Ty-CABDH | MVA.HM | 1930 | nd |

Table 5

Results of ELISPOT and CTL assays performed to measure the levels of specific CD8+ T cells to the malaria epitope pb9 following different immunisation regimes of Ty-VLPs and recombinant MVA virus as indicated. The CTL and ELISPOT data are from different experiments. The ELISPOT levels (spots per million splenocytes) are measured on un-restimulated cells and the CTL activity, indicated as specific lysis at an effector to target ratio of 40:1, on cells restimulated with pb9 peptide in vitro for 5-7 days. Both represent mean levels of three mice. BALB/c mice were used in all cases. The immunisation doses were 50 μg of Ty-VLP or 10⁷ ffu (foci forming units) of recombinant MVA. All immunisations were intramuscular. The interval between immunisations 1 and 2 was from 14-21 days. MVA.HM includes cassettes CAB.

Priming of an Immune Response with DNA Delivered by a Gene Gun and Boosting with Recombinant MVA

Immunogenicity and Challenge

The use of a gene gun to deliver plasmid DNA intradermally and thereby prime an immune response that could be boosted with recombinant MVA was investigated. Groups of BALB/c mice were immunised with the following regimen:

I) Three gene gun immunisations with pTH.PbCSP (4 mg per immunisation) at two week intervals

II) Two gene gun immunisations followed by MVA i.v. two weeks later

III) One intramuscular DNA immunisation followed by MVA i.v. two weeks later.

The immunogenicity of the three immunisation regimens was analysed using ELISPOT assays. The highest frequency of specific T cells was observed with two gene gun immunisations followed by an MVA i.v. boost and the intramuscular DNA injection followed an MVA i.v. boost (FIG. 6).

FIG. 6 shows the results of ELISPOT assays performed to measure the levels of specific CD8+ T cells to the malaria epitope pb9 following different immunisation regimes. Groups of BALB/c mice (n=3) were immunised as indicated (g.g.=gene gun). The time between all immunisations was 14 days. ELISPOT assays were done two weeks after the last immunisation.

CTL Induction to the Same Antigen in Different Mouse Strains

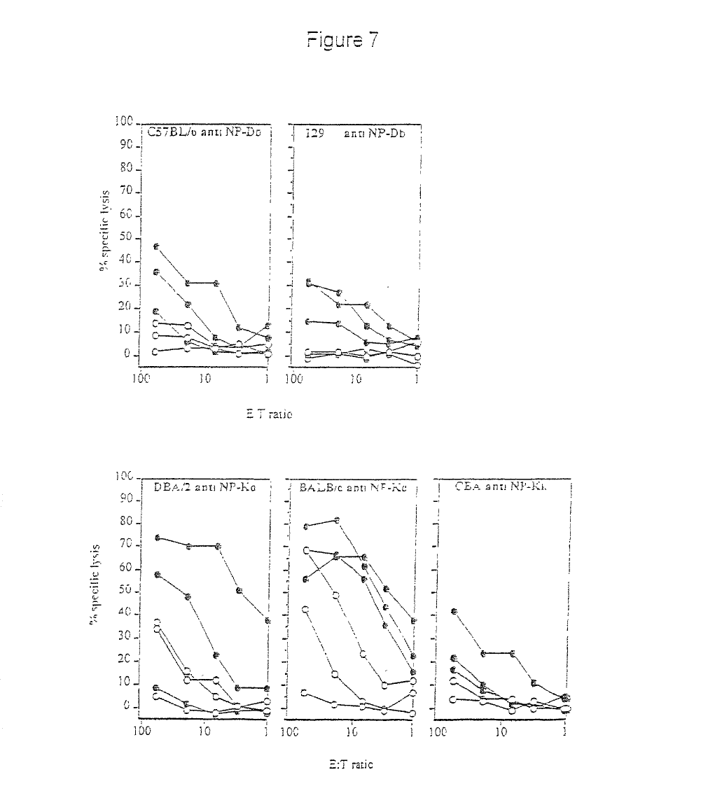

To address the question whether the boosting effect described above in BALB/c mice with two CTL epitopes SYIPSAEKI [SEQ ID NO: 67] derived from P. berghei CSP and RGPGRAFVTI [SEQ ID NO: 68] derived from HIV is a universal phenomenon, two sets of experiments were carried out. CTL responses to the influenza nucleoprotein were studied in five inbred mouse strains. In a first experiment three published murine CTL epitopes derived from the influenza nucleoprotein were studied (see Table 3). Mice of three different H-2 haplotypes, BALB/c and DBA/2 (H-2^{d}), C57BL/6 and 129 (H-2^{b}); CBA/J (H-2^{k}), were used. One set of animals was immunised twice at two week intervals with the plasmid V1J-NP encoding the influenza nucleoprotein. Another set of identical animals was primed with V1J-NP and two weeks later boosted intravenously with 10⁶ ffu of MVA.NP, expressing influenza virus NP. The levels of CTL in individual mice were determined in a ⁵¹Cr-release assay with peptide re-stimulated splenocytes. As shown in FIG. 7, the DNA priming/MVA boosting immunisation regimen induced higher levels of lysis in all the mouse strains analysed and is superior to two DNA injections.

FIG. 7 shows the CTL responses against influenza NP in different mouse strains. Mice of different strains were immunised twice two weeks apart with a DNA vaccine V1J-NP encoding for the influenza nucleoprotein (open circles) or primed with the same DNA vaccine and two weeks later boosted with recombinant MVA expressing influenza virus nucleoprotein (closed circles). Two weeks after the last immunisation splenocytes were restimulated in vitro with the respective peptides (Table 3). The CTL activity was determined in a standard ⁵¹-Cr-release assay with MHC class I-matched target cells.

CTL Induction to Different Antigens in Different Mouse Strains

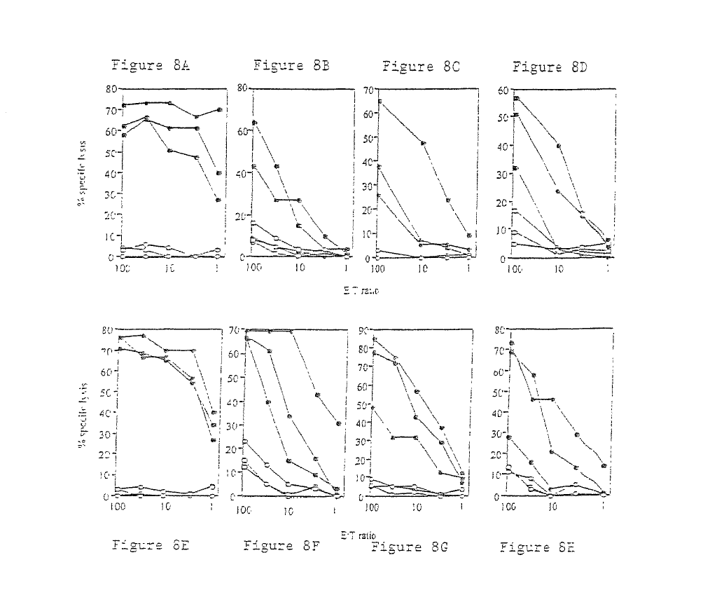

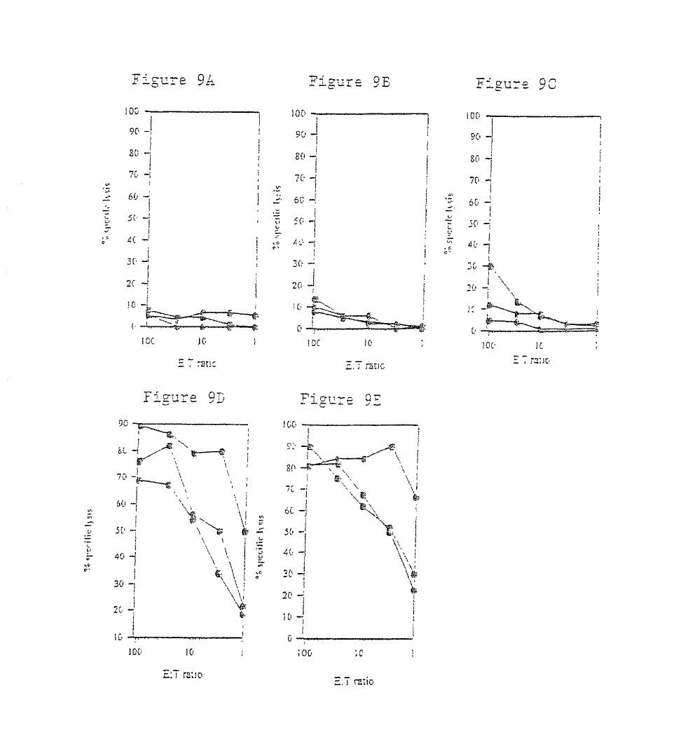

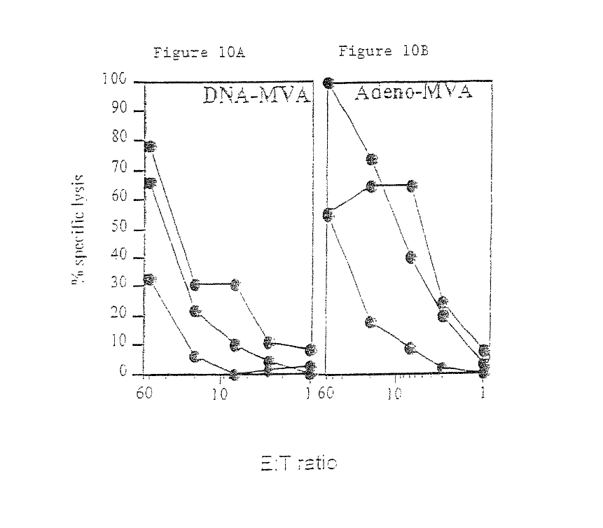

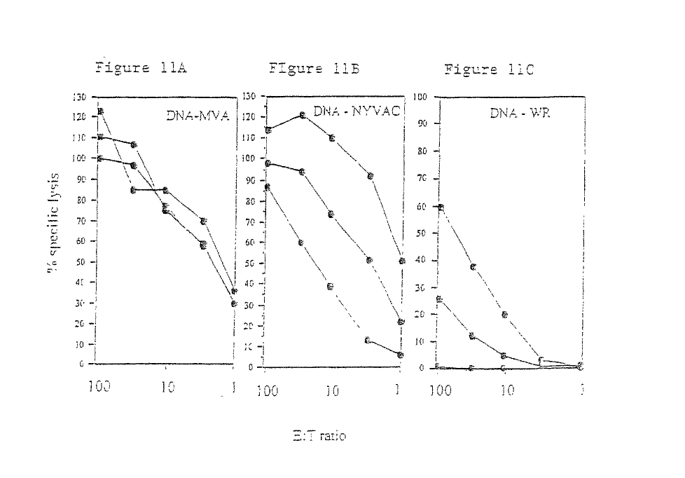

The effect of MVA boosting on plasmid DNA-primed immune responses was further investigated using different antigens and different inbred mouse strains. Mice of different strains were immunised with different antigens using two DNA immunisations and compared with DNA/MVA immunisations. The antigens used were E. coli β-galactosidase, the malaria/HIV epitope string, a murine tumour epitope string and P. falciparum TRAP. Compared with two DNA immunisations the DNA-priming/MVA-boosting regimen induced higher levels of CTL in all the different mouse strains and antigen combinations tested (FIGS. 8A-8H).

FIGS. 8A-8H show CTL responses against different antigens induced in different inbred mouse strains. Mice were immunised with two DNA vaccine immunisations two weeks apart (open circles) or primed with a DNA vaccine and two weeks later boosted with a recombinant MVA expressing the same antigen (closed circles). The strains and antigens were: C57BL/6; P. falciparum TRAP in A. DBA/2; E. coli b-galactosidase in B. BALB/c; HM epitope string CTL activity against malaria peptide (pb9) in C. DBA/2; HM epitope string CTL activity against pb9 in D. BALB/c; HM epitope string CTL activity against HIV peptide in E. DBA/2; HM epitope string CTL activity against HIV peptide in F. BALB/c; tumour epitope string CTL activity against P1A-derived peptide in G. DBA/2; tumour epitope string CTL activity against P1A-derived peptide in H. Sequences of peptide epitopes are shown in table 3. Each curve shows the data for an individual mouse.

Sporozoites Can Efficiently Prime an Immune Response That Is Boostable by MVA

Humans living in malaria endemic areas are continuously exposed to sporozoite inoculations. Malaria-specific CTL are found in these naturally exposed individuals at low levels. To address the question whether low levels of sporozoite induced CTL responses can be boosted by MVA, BALB/c mice were immunised with irradiated (to prevent malaria infection) P. berghei sporozoites and boosted with MVA. Two weeks after the last immunisation splenocytes were re-stimulated and tested for lytic activity. Two injections with 50 or 300+500 sporozoites induced very low or undetectable levels of lysis. Boosting with MVA induced high levels of peptide specific CTL. MVA alone induced only moderate levels of lysis (FIGS. 9A-9E).

FIGS. 9A-9E show sporozoite-primed CTL responses are substantially boosted by MVA. Mice were immunised with two low doses (50+50) of irradiated sporozoites in FIG. 9A; two high doses (300+500) of sporozoites in FIG. 9B; mice were boosted with MVA.PbCSP following low-dose sporozoite priming in FIG. 9D; high dose sporozoite priming in FIG. 9E. CTL responses following immunisation with MVA.PbCSP are shown in FIG. 9C.

Recombinant Adenoviruses as Priming Agent

The prime-boost immunisation regimen has been exemplified using plasmid DNA and recombinant Ty-VLP as priming agent. Here an example using non-replicating adenoviruses as the priming agent is provided. Replication-deficient recombinant Adenovirus expressing E. coli β-galactosidase (Adeno-GAL) was used. Groups of BALB/c mice were immunised with plasmid DNA followed by MVA or with Adenovirus followed by MVA. All antigen delivery systems used encoded E. coli β-galactosidase. Priming a CTL response with plasmid DNA or Adenovirus and boosting with MVA induces similar levels of CTL (FIGS. 10A-10B).

FIGS. 10A-10B show CTL responses primed by plasmid DNA or recombinant Adenovirus and boosted with MVA. Groups of BALB/c mice (n=3) were primed with plasmid DNA (FIG. 10A); or recombinant Adenovirus expressing β-galactosidase (FIG. 10B). Plasmid DNA was administered intramuscularly, MVA intravenously and Adenovirus intradermally. Splenocytes were restimulated with peptide TPHPARIGL [SEQ ID NO: 69] two weeks after the last immunisation. CTL activity was tested with peptide-pulsed P815 cells.

Immunogenicity of the DNA Prime Vaccinia Boost Regimen Depends on the Replication Competence of the Strain of Vaccinia Virus Used

The prime boosting strategy was tested using different strains of recombinant vaccina viruses to determine whether the different strains with strains differing in their replication competence may differ in their ability to boost a DNA-primed CTL response. Boosting with replication-defective recombinant vaccinia viruses such as MVA and NYVAC resulted in the induction of stronger CTL responses compared to CTL responses following boosting with the same dose of replication competent WR vaccinia virus (FIGS. 11A-11C).

FIGS. 11A-11C show CTL responses in BALB/c mice primed with plasmid DNA followed by boosting with different recombinant vaccinia viruses. Animals were primed with pTH.PbCSP 50 μg/mouse i.m. and two weeks later boosted with different strains of recombinant vaccina viruses (10⁶ pfu per mouse i.v.) expressing PbCSP. The different recombinant vaccinia virus strains were MVA in FIG. 11A; NYVAC in FIG. 11B and WR in FIG. 11C. The superiority of replication-impaired vaccinia strains over replicating strains was found in a further experiment. Groups of BALB/c mice (n=6) were primed with 50 μg/animal of pSG2.PbCSP (i.m.) and 10 days later boosted i.v. with 10⁶ ffu/pfu of recombinant MVA, NYVAC and WVR expressing PbCSP. The frequencies of peptide-specific CD8+ T cells were determined using the ELISPOT assay. The frequencies were: MVA 1103+/−438, NYVAC 826+/−249 and WR 468+/−135. Thus using both CTL assays and ELISPOT assays as a measure of CD8 T cell immunogenicity a surprising substantially greater immunogenicity of the replication-impaired vaccinia strains was observed compared to the replication competent strain.

The Use of Recombinant Canary or Fowl Pox Viruses for Boosting Cd8+ T Cell Responses

Recombinant canary pox virus (rCPV) or fowl pox virus (rFVP) are made using shuttle vectors described previously (Taylor et al. Virology 1992, 187: 321-328 and Taylor et al. Vaccine 1988, 6: 504-508). The strategy for these shuttle vectors is to insert the gene encoding the protein of interest preceded by a vaccinia-specific promoter between two flanking regions comprised of sequences derived from the CPV or FPV genome. These flanking sequences are chosen to avoid insertion into essential viral genes. Recombinant CPV or FPV are generated by in vivo recombination in permissive avian cell lines i.e. primary chicken embryo fibroblasts. Any protein sequence of antigens or epitope strings can be expressed using fowl pox or canary pox virus. Recombinant CPV or FPV is characterised for expression of the protein of interest using antigen-specific antibodies or including an antibody epitope into the recombinant gene. Recombinant viruses are grown on primary CEF. An immune response is primed using plasmid DNA as described in Materials and Methods. This plasmid DNA primed immune response is boosted using 10⁷ ffu/pfu of rCPV or rFPV inoculated intravenously, intradennally or intramuscularly. CD8+ T cell responses are monitored and challenges are performed as described herein.

### Example 3

Malaria Challenge Studies in Mice

To assess the protective efficacy of the induced levels of CD8+ T cell response immunised BALB/c or C57BL/6 mice were challenged by intravenous injection with 2000 or 200 P. berghei sporozoites. This leads to infection of liver cells by the sporozoites. However, in the presence of a sufficiently strong T lymphocyte response against the intrahepatic parasite no viable parasite will leave the liver and no blood-stage parasites will be detectable. Blood films from challenged mice were therefore assessed for parasites by microscopy 5-12 days following challenge.

BALB/c mice immunised twice with a mixture of two plasmid DNAs encoding the CS protein and the TRAP antigen, respectively, of P. berghei were not protected against sporozoite challenge. Mice immunised twice with a mixture of recombinant MVA viruses encoding the same two antigens were not protected against sporozoite challenge. Mice immunised first with the two recombinant MVAs and secondly with the two recombinant plasmids were also not protected against sporozoite challenge. However, all 15 mice immunised first with the two plasmid DNAs and secondly with the two recombinant MVA viruses were completely resistant to sporozoite challenge (Table 6 A and B).

To assess whether the observed protection was due to an immune response to the CS antigen or to TRAP or to both, groups of mice were then immunised with each antigen separately (Table 6 B). All 10 mice immunised first with the CS plasmid DNA and secondly with the CS MVA virus were completely protected against sporozoite challenge. Fourteen out of 16 mice immunised first with the TRAP plasmid DNA vaccine and secondly with the TRAP MVA virus were protected against sporozoite challenge. Therefore the CS antigen alone is fully protective when the above immunisation regime is employed and the TRAP antigen is substantially protective with the same regime.

The good correlation between the induced level of CD8+ T lymphocyte response and the degree of protection observed strongly suggests that the CD8+ response is responsible for the observed protection. In previous adoptive transfer experiments it has been demonstrated that CD8+ T lymphocyte clones against the major CD8+ T cell epitope in the P. berghei CS protein can protect against sporozoite challenge. To determine whether the induced protection was indeed mediated by CD8+ T cells to this epitope we then employed a plasmid DNA and a recombinant MVA encoding only this nine amino acid sequence from P. berghei as a part of a string of epitopes (Table 6 B). (All the other epitopes were from micro-organisms other than P. berghei). Immunisation of 10 mice first with a plasmid encoding such an epitope string and secondly with a recombinant MVA also encoding an epitope string with the P. berghei CTL epitope led to complete protection from sporozoite challenge (Table 6 B). Hence the induced protective immune response must be the CTL response that targets this nonamer peptide sequence.

TABLE 6
Results of Mouse Challenge Experiments Using Different Combinations
of DNA and MVA Vaccine

| | | | |
|---|---|---|---|
| | | No. Infected/ | |
| Immunisation 1 | Immunisation 2 | No. challenged | % Protection |

A. Antigens used: PbCSP + PbTRAP

| | | | |
|---|---|---|---|
| DNA | DNA | 5/5 | 0% |
| MVA | MVA | 9/10 | 10% |
| DNA | MVA | 0/5 | 100% |
| MVA | DNA | 5/5 | 0% |

Control mice immunised with p-galactosidase

| | | | |
|---|---|---|---|
| DNA | DNA | 5/5 | 0% |
| MVA | MVA | 5/5 | 0% |
| DNA | MVA | 5/5 | 0% |
| MVA | DNA | 5/5 | 0% |

B.

| | | | |
|---|---|---|---|
| DNA (CSP ± | MVA (CSP ± TRAP) | 0/10 | 100% |
| TRAP) | | | |
| DNA (CSP) | MVA (CSP) | 0/10 | 100% |
| DNA (TRAP) | MVA (TRAP) | 2/16 | 88% |
| DNA (epitope) | MVA (epitope) | 0/11 | 100% |
| DNA (beta-gal) | MVA (beta-gal) | 6/7 | 14% |
| none | none | 9/10 | 10% |

Table 6

Results of Two Challenge Experiments (A and B) Using Different Immunisation regimes of plasmid DNA and MVA as indicated. BALB/c mice were used in all cases. The immunisation doses were 50 μg of plasmid DNA or 10⁶ ffu of recombinant MVA. The interval between immunisations 1 and 2 was from 14-21 days in all cases. Challenges were performed at 18-29 days after the last immunisation by i.v. injection of 2000 P. berghei sporozoites and blood films assessed at 5, 8 and 10 days post challenge. CSP and TRAP indicate the entire P. berghei antigen and ‘epitope’ indicates the cassettes of epitopes shown in table 1 containing only a single P. berghei K^{d}-restricted nonamer CTL epitope. Note that in experiment B immunisation with the epitope string alone yields 100% protection.

Mice immunised twice with recombinant Ty-VLPs encoding pb9 were fully susceptible to infection. Similarly mice immunised twice with the recombinant MVA encoding the full CS protein were fully susceptible to infection. However, the mice immunised once with the Ty-VLP and subsequently once with the recombinant MVA showed an 85% reduction in malaria incidence when boosted with MVA expressing the full length CS protein, and 95% when MVA expressing the HM epitope string which includes pb9 was used to boost (Table 7).

TABLE 7
Results of Challenge Experiments Using Different Immunisation
Regimes of Ty-VLPs and MVA

| | | No. Infected/ | |
|---|---|---|---|
| Immunisation 1 | Immunisation 2 | No. challenged | % Protection |
| Ty-CABDHFE | Ty-CABDHFE | 7/8 | 13% |
| Ty-CABDH | MVA.PbCSP | 2/13 | 85% |
| Ty-CABDHFE | MVA-NP | 5/5 | 0% |
| MVA.PbCSP | MVA.PbCSP | 6/6 | 0% |
| MVA.HM | Ty-CABDHFE | 14/14 | 0% |
| Ty-CABDHFE | MVA.HM | 1/21 | 95% |
| none | MVA.HM | 8/8 | 0% |
| none | none | 11/12 | 9% |

Table 7

Results of Challenge Experiments Using Different Immunisation Regimes of Ty-VLPs and MVA as Indicated. BALb/c Mice Were Used in All Cases.

Immunisations were of 50 μg of Ty-VLP or 10⁷ ffu of recombinant MVA administered intravenously. The interval between immunisations 1 and 2 was from 14-21 days in all cases. Challenges were performed at 18-29 days after the last immunisation by i.v. injection of 2000 P. berghei sporozoites and blood films assessed at 5, 8 and 10 days post challenge. CSP indicates the entire P. berghei antigen. Ty-VLPs carried epitope cassettes CABDH or CABDHFE as described in table 1. MVA.HM includes cassettes CAB.

To determine whether the enhanced immunogenicity and protective efficacy observed by boosting with a recombinant MVA is unique to this particular vaccinia virus strain or is shared by other recombinant vaccinias the following experiment was performed. Mice were immunised with the DNA vaccine encoding P. berghei CS protein and boosted with either (i) recombinant MVA encoding this antigen; (ii) recombinant wild-type vaccinia virus (Western Reserve strain) encoding the same antigen (Satchidanandam et al., Plasmodium berghei. Mol. Biochem. Parasitol., 48: 89-99 (1991)), or (iii) recombinant NYVAC (COPAK) virus (Lanar et al., Infection and Immunity, 64: 1666-71 (1996)) encoding the same malaria antigen. The highest degree of protection was observed with boosting by the MVA recombinant, 80% (Table 8). A very low level of protection (10%) was observed by boosting with the wild-type recombinant vaccinia virus and a significant level of protection, 60%, by boosting with the NYVAC recombinant. Hence the prime-boost regime we describe induces protective efficacy with any non-replicating vaccinia virus strain. Both the MVA recombinant and NYVAC were significantly (P<0.05 for each) better than the WR strain recombinant.

TABLE 8
Challenge Data Results for DNA Boosted with
Various Vaccinia Strain Recombinants.

| | | No. Infected/ | |
|---|---|---|---|
| Immunisation 1 | Immunisation 2 | No. challenged | % Protection |
| DNA-beta gal. | MVA.NP | 8/8 | 0% |
| DNA-CSP | MVA-CSP | 2/10 | 80% |
| DNA-CSP | WR-CSP | 9/10 | 10% |
| DNA-CSP | NYVAC-CSP | 4/10 | 60% |

Table 8

Results of a challenge experiment using different immunisation regimes of plasmid DNA and various vaccinia recombinants as indicated. BALB/c mice were used in all cases. The immunisation doses were 50 μg of plasmid DNA or 10⁶ ffu/pfu of recombinant MVA or 10⁴ ffu/pfu of recombinant wild type (WR) vaccinia or 10⁶ ffu/pfu of recombinant NYVAC. Because the WR strain will replicate in the host and the other strains will not, in this experiment a lower dose of WR was used. The interval between immunisations 1 and 2 was 23 days. Challenges were performed at 28 days after the last immunisation by i.v. injection of 2000 P. berghei sporozoites and blood films assessed at 7, 9 and 11 days post challenge. pbCSP indicates the entire P. berghei antigen and NP the nucleoprotein antigen of influenza virus (used as a control antigen). The first immunisation of group A mice was with the plasmid DNA vector expressing beta galactosidase but no malaria antigen.

In a further experiment shown in Table 8, mice were immunised with the DNA vaccine encoding P. berghei CS protein and boosted with either (i) recombinant MVA encoding this antigen; (ii) recombinant WR vaccinia virus encoding the same antigen or (iii) recombinant NYVAC (COPAK) virus encoding the same malaria antigen, all at 10⁶ ffu/pfu. A high and statistically significant degree of protection was observed with boosting with recombinant NYVAC (80%) or recombinant MVA (66%). A low and non-significant level of protection (26%) was observed by boosting with the WR recombinant vaccinia virus (Table 9). MVA and NYVAC boosting each gave significantly more protection than WR boosting (P=0.03 and P=0.001 respectively). These data reemphasise that non-replicating pox virus strains are better boosting agents for inducing high levels of protection.

TABLE 9
Influence of Different Recombinant Vaccinia Strains on Protection.

| Immunisation 1 | | No. inf./ | % |
|---|---|---|---|
| DNA | Immunisation 2 | No. chall. | protection |
| CSP | MVA.PbCSP | 5/15 | 66 |
| CSP | NYVAC.PbCSP | 2/15 | 80 |
| CSP | WR.PbCSP | 11/15 | 26 |
| β-galactosidase | MVA.NP | 8/8 | 0 |

Table 9

Results of challenge experiments using different immunisation regimes of plasmid DNA and replication incompetent vaccinia recombinants as boosting immunisation. BALB/c mice were used in all cases. The immunisation doses were 50 μg of plasmid DNA or 10⁶ ffu/pfu of recombinant MVA or recombinant wild type (WR) vaccinia or recombinant NYVAC. The interval between immunisations 1 and 2 was 23 days. Challenges were performed at 28 days after the last immunisation by i.v. injection of 2000 P. berghei sporozoites and blood films assessed at 7, 9 and 11 days post challenge. PbCSP indicates the entire P. berghei antigen and NP the nucleoprotein antigen of influenza virus (used as a control antigen). The control immunisation was with a plasmid DNA vector expressing β-galactosidase followed by MVA.NP.

Alternative Routes for Boosting Immune Responses with Recombinant MVA

Intravenous injection of recombinant MVA is not a preferred route for immunising humans and not feasible in mass immunisations. Therefore different routes of MVA boosting were tested for their immunogenicity and protective efficacy.

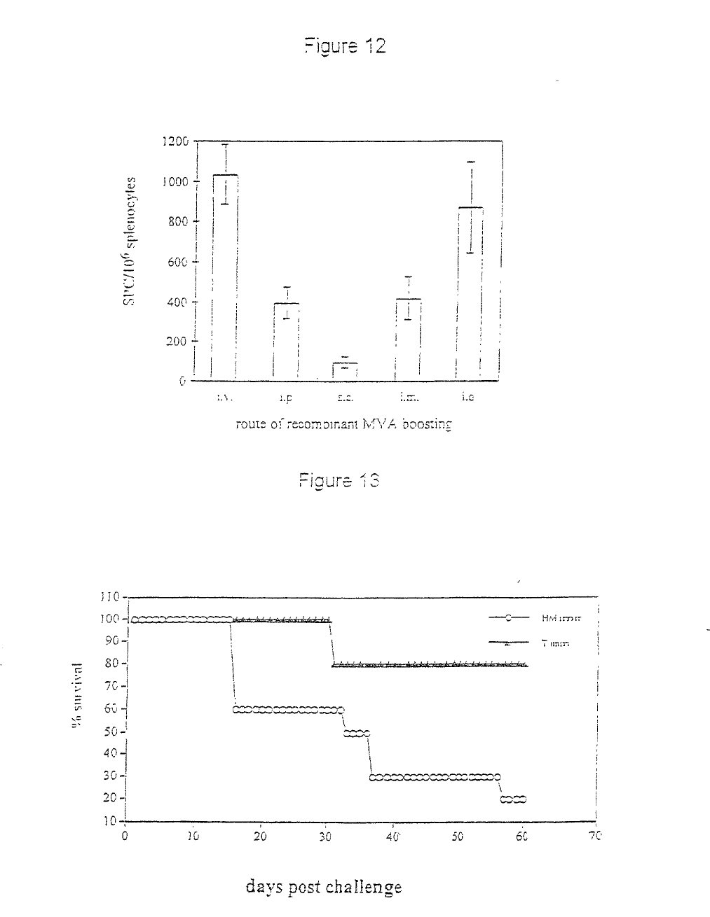

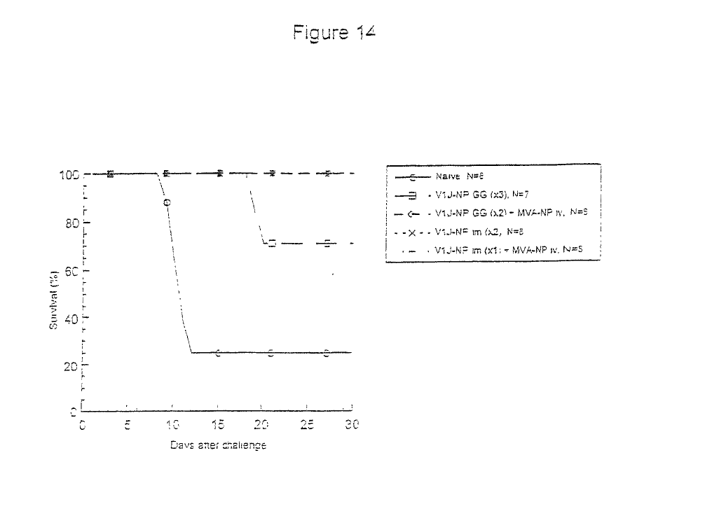

Mice were primed with plasmid DNA i.m. Two weeks later they were boosted with MVA administered via the following routes: intravenous (i.v.), subcutaneous (s.c.), intraperitoneal (i.p.), intramuscular (i.p.) and intradermal (i.d.). Two weeks after this boost peptide-specific CD8+ T cells were determined in an ELISPOT assay. The most effective route which induced the highest levels were i.v. and i.d inoculation of MVA. The other routes gave moderate to poor responses (FIG. 12).

FIG. 12 shows frequencies of peptide-specific CD8+ T cells following different routes of MVA boosting. Results are shown as the number of spot-forming cells (SFC) per one million splenocytes. Mice were primed with plasmid DNA and two weeks later boosted with MVA via the indicated routes. The number of splenocytes specific for the SYIPSAEKI [SEQ ID NO: 67] peptide was determined in INF-γ ELISPOT assays two weeks after the last immunisation. Each bar represents the mean number of SFCs from three mice assayed individually.

Boosting via the i.v. route was compared with the i.d. and i.m route in a challenge experiment. The i.d route gave high levels of protection (80% protection). In the group of animals that were boosted via the i.m. route, 50% of the animals were protected. Complete protection was achieved with MVA boost administered i.v. (Table 10)

TABLE 10
Influence of the Route of MVA Administration on Protective Efficacy

| | | No. | |
|---|---|---|---|
| Immunisation | Immunisation | infected/ | |
| 1 | 2 | No. | |
| DNA | MVA | challenged | % protection |
| CSP | CSP i.v. | *0/20 | 100 |
| CSP | CSP i.d | 2/10 | 80 |
| CSP | CSP i.m. | 5/10 | 50 |
| Epitope | epitope i.v. | 1/10 | 90 |
| NP | NP i.v. | 10/10 | 0 |

*culminative data from two independent experiments

Table 10

Results from challenge experiments using different routes of MVA boosting immunisation. Animals were primed by intramuscular plasmid DNA injection and two weeks later boosted with the indicated recombinant MVA (10⁶ ffu/mouse) administered via the routes indicated. The mice were challenged 16 days after the last immunisation with 2000 P. berghei sporozoites and screened for blood stage parasitemia at day 8 and 10 post challenge. Epitope indicates the polypeptide string HM.

Alternative Routes of DNA Priming: The Use of a Gene Gun to Prime Peptide Specific Cd8+ T Cells

Gene gun delivery is described in detail in for example in Eisenbraun et al. DNA Cell Biol. 1993, 12: 791-797 and Degano et al. Vaccine 1998, 16: 394-398.

The mouse malaria challenge experiments described so far using plasmid DNA to prime an immune response used intramuscular injection of plasmid DNA. Intradermal delivery of plasmid DNA using a biolistic device is another route to prime specific CTL responses. Plasmid DNA is coated onto gold particles and delivered intradermally with a gene gun. Groups of mice (n=10) were immunised three times at two weeks intervals with the gene gun alone (4 μg/immunisation), immunised two times with the gene gun followed by an intravenous MVA.PbCSP boost or immunised intramuscularly with 50 μg of pTH.PbCSP and two weeks later boosted with MVA.PbCSP intravenously. Two weeks after the last immunisation the animals were challenged with 2000 sporozoites to assess protective efficacy of each immunisation regimen. In the group that received the intravenous MVA boost following two gene gun immunisations one out of ten animals developed blood stage parasitemia (90% protection). Complete protection was observed with intramuscular DNA priming followed by MVA i.v boosting. Seven out of 10 animals that were immunised three times with the gene gun were infected. (30% protection) (Table 11).

| Immunisation 1 | | | No. inf./ | % |
|---|---|---|---|---|
| DNA | Immunisation 2 | Immunisation 3 | No. chall. | protection |
| gene gun DNA | gene gun DNA | gene gun DNA | 7/10 | 30 |
| gene gun DNA | gene gun DNA | MVA.PbCSP | 1/10 | 90 |
| — | DNA i.m | MVA.PbCSP | 0/10 | 100 |
| Naïve | | | 10/10 | 0 |

Table 11

Results of challenge experiments comparing different routes of DNA priming (intradermally by gene gun versus intramuscular needle injection). Groups of BALB/c mice (n=10) were immunised as indicated. Each gene gun immunisation delivered 4 μg of plasmid DNA intraepidermally. For i.m. immunisations 50 mg of plasmid DNA were injected. Twenty days after the last immunisation mice were challenged as described previously.

Highly Susceptible C57BL/6 Mice Are Protected

C57BL/6 mice are very susceptible to P. berghei sporozoite challenge. C57BL/6 mice were immunised using the DNA-MVA prime boost regime with both pre-erythrocytic antigens PbCSP and PbTRAP, and challenged with either 200 or 1000 infectious sporozoites per mouse. (Two hundred sporozoites corresponds to more than twice the dose required to induce infection in this strain). All ten mice challenged with 200 sporozoites showed sterile immunity. Even the group challenged with 1000 sporozoites, 60% of the mice were protected (Table 12). All the naïve C57BL/6 mice were infected after challenge.

TABLE 12
Protection of C57BL/6 Mice from Sporozoite Challenge

| | No. animals inf./ | % |
|---|---|---|
| | No. challenged | protection |
| 1000 sporozoites | | |
| DNA followed by MVA | 4/10 | 60 |
| Naïve | 5/5 | 0 |
| 200 sporozoites | | |
| DNA followed by MVA | 0/10 | 100 |
| Naïve | 5/5 | 0 |

Table 12

Results of a challenge experiment using C57BL/6 mice. Animals were immunised with PbCSP and PbTRAP using the DNA followed by MVA prime boost regime. Fourteen days later the mice were challenged with P. berghei sporozoites as indicated.

### Example 4

Protective Efficacy of the DNA-priming/MVA-Boosting Regimen in Two Further Disease Models in Mice

Following immunogenicity studies, the protective efficacy of the DNA-priming MVA-boosting regimen was tested in two additional murine challenge models. The two challenge models were the P815 tumour model and the influenza A virus challenge model. In both model systems CTL have been shown to mediate protection.

P815 Tumour Challenges:

Groups (n=10) of DBA/2 mice were immunised with a combination of DNA followed by MVA expressing a tumour epitope string or the HM epitope string. Two weeks after the last immunisation the mice were challenged intravenously with 10⁵ P815 cells. Following this challenge the mice were monitored regularly for the development of tumour-related signs and survival.

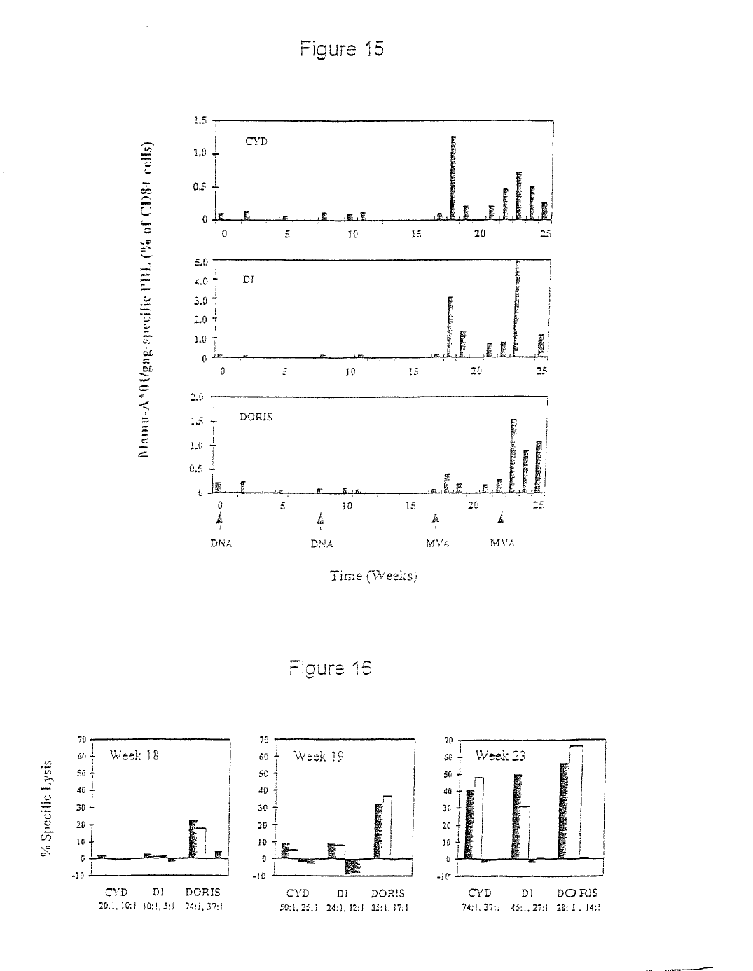

FIG. 13 shows the survival rate of the two groups of mice. Sixty days after challenge eight out of ten mice were alive in the group immunised with the tumour epitopes string. In the group immunised with the HM epitope string only 2 animals survived. This result is statistically significant: 2/10 vs 8/10 chi-squared=7.2. P=0.007. The onset of death in the groups of animals immunised with the tumour epitope string is delayed compared to the groups immunised with the HM epitope string.

Influenza Virus Challenges:

Groups of BALB/c mice were immunised with three gene gun immunisations with plasmid DNA, two intramuscular plasmid DNA injections, one i.m. DNA injection followed by one MVA.NP boost i.v. or two gene gun immunisations followed by one MVA.NP boost i.v. Plasmid DNA and recombinant MVA expressed the influenza virus nucleoprotein. Two weeks after the last immunisation the mice were challenged intranasally with 100 HA of influenza A/PR/8/34 virus. The animals were monitored for survival daily after challenge.

Complete protection was observed in the following groups of animals:

two DNA gene gun immunisations followed by one MVA.NP boost i.v.;

one i.m. DNA injection followed by one MVA.NP boost i.v.; and

two i.m. DNA injections.

In the group of animals immunised three times with the gene gun 71% of the animals survived (5/7) and this difference from the control group was not significant statistically (P>0.05). In the naive group 25% of the animals survived (FIG. 14) and this group differed significantly (P<0.05) for the two completely protected groups.

FIG. 14 shows results of an influenza virus challenge experiment. BALB/c mice were immunised as indicated. GG=gene gun immunisations, im=intramuscular injection, iv=intravenous injection. Survival of the animals was monitored daily after challenge. In a second experiment groups of 10 BALB/c mice were immunised with MVA.NP i.v. alone, three times with the gene gun, two times with the gene gun followed by one MVA.NP boost i.v. and two i.m injections of V1J-NP followed by one MVA.NP boost. Two weeks after the last immunisation the mice were challenged with 100 HA units of influenza A/PR/8/34 virus.

Complete and statistically significant protection was observed in the following groups of animals:

two gene gun immunisations followed by one MVA.NP boost; and

two i.m injections of V1J-NP followed by one MVA.NP boost.

In the group receiving one MVA.NP i.v., 30% (3 out of 10) of animals survived. In the group immunised with a DNA vaccine delivered by the gene gun three times, 70% of the animals were protected but this protection was not significantly different from the naïve controls. In this challenge experiment 40% (4 out of 10) of the naive animals survived the challenge.

### Example 5

Immunogenicity Studies in Non-human Primates

Immunogenicity and Protective Efficacy of the Prime Boost Regimen in Non-human Primates

In order to show that the strong immunogenicity of the DNA priming/MVA boosting regime observed in mice translates into strong immunogenicity in primates, the regimen was tested in macaques. The vaccine consisted of a string of CTL epitopes derived from HIV and SIV sequences (FIG. 2), in plasmid DNA or MVA, denoted DNA.H and MVA.H respectively. The use of defined CTL epitopes in a polyepitope string allows testing for SIV specific CTL in macaques. Due to the MHC class I restriction of the antigenic peptides, macaques were screened for their MHC class I haplotype and Mamu-A*01-positive animals were selected for the experiments described.

Three animals (CYD, DI and DORIS) were immunised following this immunisation regimen:

| | |
|---|---|
| week 0 | DNA (8 μg, i.d., gene gun) |
| week 8 | DNA (8 μg, i.d., gene gun) |
| week 17 | MVA (5 × 10⁸ pfu, i.d.) |
| week 22 | MVA (5 × 10⁸ pfu, i.d.) |

Blood from each animal was drawn at weeks 0, 2, 5, 8, 10, 11, 17, 18, 19, 21, 22, 23, 24 and 25 of the experiment. The animals were monitored for induction of CTL using two different methods. PBMC isolated from each bleed were re-stimulated in vitro with a peptide encoded in the epitope string and tested for their ability to recognise autologous peptide-loaded target cells in a chromium release cytotoxicity assay. Additionally, freshly isolated PBMC were stained for antigen specific CD8+ T cells using tetramers.

Following two gene gun immunisations very low levels of CTL were detected using tetramer staining (FIG. 15). Two weeks after the first MVA boosting, all three animals developed peptide specific CTL as detected by tetramer staining (FIG. 15). This was reflected by the detection of moderate CTL responses following in vitro restimulation (FIG. 16, week 19). The second boost with MVA.H induced very high levels of CD8+, antigen specific T cells (FIG. 15) and also very high levels of peptide specific cytotoxic T cells (FIG. 16, week 23).

FIG. 15 shows detection of SIV-specific MHC class I-restricted CD8+ T cells using tetramers. Three Mamu-A*A01-positive macaques were immunised with plasmid DNA (gene gun) followed by MVA boosting as indicated. Frequencies of Mamu-A*A01/CD8 double-positive T cells were identified following FACS analysis. Each bar represents the percentage of CD8+ T cells specific for the Mamu-A*01/gag epitope at the indicated time point. One percent of CD8 T cells corresponds to about 5000/10⁶ peripheral blood lymphocytes. Thus the levels of epitope-specific CD8 T cells in the peripheral blood of these macaques are at least as high as the levels obvserved in the spleens of immunised and protected mice in the malaria studies.

FIG. 16 shows CTL induction in macaques following DNA/MVA immunisation. PBMC from three different macaques (CYD, DI and DORIS) were isolated at week 18, 19 and 23 and were restimulated with peptide CTPYDINQM [SEQ ID NO: 54] in vitro. After two restimulations with peptide CTPYDINQM [SEQ ID NO: 54] the cultures were tested for their lytic activity on peptide-pulsed autologous target cells. Strong CTL activity was observed.

### Example6

Immunogenicity and Challenge Studies in Chimpanzees

To show that a similar regime of initial immunisation with plasmid DNA and subsequent immunisation with recombinant MVA can be effective against Plasmodium falciparum malaria in higher primates an immunisation and challenge study was performed with two chimpanzees. Chimp H1 received an initial immnunisation with 500 μg of a plasmid expressing Plasmodium falciparum TRAP from the CMV promoter without intron A, CMV-TRAP. Chimp H2 received the same dose of CMV-LSA-1, which expresses the C-terminal portion of the LSA-1 gene of P. falciparum. Both chimps received three more immunisations over the next 2 months, but with three plasmids at each immunisation. H1 received CMV-TRAP as before, plus pTH-TRAP, which expresses TRAP using the CMV promoter with intron A, leading to a higher expression level. H1 also received RSV-LSA-1, which expresses the C-terminal portion of LSA-1 from the RSV promoter. H2 received CMV-LSA-1, pTH-LSA-1 and RSV-TRAP at the second, third and fourth immunisations. The dose was always 500 μg of each plasmid.

It was subsequently discovered that the RSV plasmids did not express the antigens contained within them, so H1 was only immunised with plasmids expressing TRAP, and H2 with plasmids expressing LSA-1.

Between and following these DNA immunisations assays of cellular immune responses were performed at several time points, the last assay being performed at three months following the fourth DNA immunisation, but no malaria-specific T cells were detectable in either ELISPOT assays or CTL assays for CD8+ T cells.

Both animals were subsequently immunised with three doses of 10⁸ ffu of a recombinant MVA virus encoding the P. falciparum TRAP antigen over a 6 week period. Just before and also following the third recombinant MVA immunisation T cell responses to the TRAP antigen were detectable in both chimpanzees using an ELISPOT assay to whole TRAP protein bound to latex beads. This assay detects both CD4+ and CD8+ T cell responses. Specific CD8+ T responses were searched for with a series of short 8-11 amino acid peptides in both immunised chimpanzees. Such analysis for CD8+ T cell responses indicated that CD8+ T cells were detectable only in the chimpanzee H1. The target epitope of these CD8+ T lymphocytes was an 11 amino acid peptide from TRAP, tr57, of sequence KTASCGVWDEW [SEQ ID NO: 78]. These CD8+ T cells from H1 had lytic activity against autologous target cells pulsed with the tr57 peptide and against autologous target cells infected with the recombinant PfTRAP-MVA virus. A high precursor frequency of these specific CD8+ T cells of about 1 per 500 lymphocytes was detected in the peripheral blood of this chimpanzee H1 using an ELISPOT assay two months following the final MVA immunisation. No specific CD8+ T cell response was clearly detected in the chimpanzee H2, which was not primed with a plasmid DNA expressing TRAP.

Two months after the third PfTRAP-MVA immunisation challenge of H1 and H2 was performed with 20,000 sporozoites, a number that has previously been found to yield reliably detectable blood stage infection in chimpanzees 7 days after challenge (Thomas et al., Mem. Inst. Oswaldo Cruz., 89 Suppl 2: 111-4 (1994) and unpublished data). The challenge was performed with the NF54 strain of Plasmodium falciparum. This is of importance because the TRAP sequence in the plasmid DNA and in the recombinant MVA is from the T9/96 strain of P. falciparum which has numerous amino acid differences to the NF54 TRAP allele (Robson et al., 1990). Thus, this sporozoite challenge was performed with a heterologous rather than homologous strain of parasite. In the chimpanzee H2 parasites were detectable in peripheral blood as expected 7 days after sporozoite challenge using in vitro parasite culture detection. However, in H1 the appearance of blood stage parasites in culture from the day 7 blood samples was delayed by three days consistent with some immune protective effect against the liver-stage infection. In studies of previous candidate malaria vaccines in humans a delay in the appearance of parasites in the peripheral blood has been estimated to correspond to a substantial reduction in parasite density in the liver (Davis et al., Transactions of the Royal Society for Tropical Medicine and Hygiene., 83: 748-50 (1989)). Thus the chimpanzee H1, immunised first with P. falciparum TRAP plasmid DNA and subsequently with the same antigen expressed by a recombinant MVA virus showed a strong CD8+ T lymphocyte response and evidence of some protection from heterologous sporozoite challenge.

Discussion

These examples demonstrate a novel regime for immunisation against malaria which induces high levels of protective CD8+ T cells in rodent models of human malaria infection. Also demonstrated is an unprecedented complete protection against sporozoite challenge using subunit vaccines (36 out of 36 mice protected in Table 6 using DNA priming and MVA boosting with the CS epitope containing vaccines). Induction of protective immune responses using the DNA priming/MVA boosting regimen was demonstrated in two additional mouse models of viral infection influenza A model and cancer (P815 tumour model). More importantly for vaccines for use in humans this immunisation regimen is also highly immunogenic for CD8+ T cells in primates. Strong SIV-gag-specific CTL were induced in 3 out of 3 macaques with plasmid DNA and MVA expressing epitope strings. The levels induced are comparable to those found in SIV-infected animals. The data from the chimpanzee studies indicate that the same immunisation regime can induce a strong CD8+ T lymphocyte response against P. falciparum in higher primates with some evidence of protection against P. falciparum sporozoite challenge.

Ty-VLPs have previously been reported to induce good levels of CD8+ T cell responses against the P. berghei rodent malaria (Allsopp et al., European Journal of Immunology, 26:1951-1959 (1995)) but alone this construct is not protective. It has now been found that subsequent immunisation with recombinant MVA boosts the CD8+ T cell response very substantially and generates a high level of protection (Table 7).

Recombinant MVA viruses have not been assessed for efficacy as malaria vaccines previously. Recombinant MVA alone was not significantly protective, nor was priming with recombinant MVA followed by a second immunisation with recombinant plasmid DNA. However, a second immunisation with the recombinant MVA following an initial immunisation with either Ty-VLPs or plasmid DNA yielded impressive levels of protection. Non-recombinant MVA virus has been safely used to vaccinate thousands of human against smallpox and appears to have an excellent safety profile. The molecular basis of the increased safety and immunogenicity of this strain of vaccinia virus is being elucidated by detailed molecular studies (Meyer et al., J Gen Virol., 72: 1031-8 (1991); Sutter et al., J. Virol., 68: 4109-16. (1994); Sutter et al., Vaccine, 12: 1032-40 (1994)).

Plasmid DNA has previously been tested as a malaria vaccine for the P. yoelii rodent malaria. High levels of, but not complete, protection is seen in some strains but in other strains of mice little or no protection was observed even after multiple immunisations (Doolan et al., J. Exp. Med., 183: 1739-46 (1996)). Although plasmid DNA has been proposed as a method of immunisation against P. falciparum, success has not previously been achieved. The evidence provided here is the first evidence to show that plasmid DNA may be used in an immunisation regime to induce protective immunity against the human malaria parasite P. falciparum.

A similar regime of immunisation to the regime demonstrated herein can be expected to induce useful protective immunity against P. falciparum in humans. It should be noted that five of the vaccine constructs employed in these studies to induce protective immunity in rodents or chimpanzees contain P. falciparum sequences and could therefore be used for human immunisation against P. falciparum. These are: 1. The P. falciparum TRAP plasmid DNA vaccine. 2. The P. falciparum TRAP recombinant MVA virus. 3. The Ty-VLP encoding an epitope string of numerous P. falciparum epitopes, as well as the single P. berghei CTL epitope. 4. The plasmid DNA encoding the same epitope string as 3. 5. The recombinant MVA encoding the longer HM epitope string including many of the malaria epitopes in 3 and 4. Similarly the plasmid DNAs and MVA encoding HIV epitopes for human class I molecules could be used in either prophylactic or therapeutic immunisation against HIV infection.

These studies have provided clear evidence that a novel sequential immunisation regime employing a non-replicating or replication-impaired pox virus as a boost is capable of inducing a strong protective CD8+ T cell response against the malaria parasite. The examples demonstrate clearly a surprising and substantial enhancement of CD8+ T cell responses and protection compared to replicating strains of pox viruses. Because there is no reason to believe that the immunogenicity of CD8+ T cell epitopes from the malaria parasite should differ substantially from CD8+ T cell epitopes in other antigens it is expected that the immunisation regime described herein will prove effective at generating CD8+ T cell responses of value against other diseases. The critical step in this immunisation regimen is the use of non-replicating or replication-impaired recombinant poxviruses to boost a pre-existing CTL response. We have shown that CTL responses can be primed using different antigen delivery systems such as a DNA vaccine i.d. and i.m, a recombinant Ty-VLP, a recombinant adenovirus and irradiated sporozoites. This is supported by the data presented on the generation of a CD8+ T cell response against HIV, influenza virus and tumours. Amongst several known examples of other diseases against which a CD8+ T cell immune response is important are the following: infection and disease caused by the viruses HIV, herpes simplex, herpes zoster, hepatitis C, hepatitis B, influenza, Epstein-Barr virus, measles, dengue and HTLV-1; by the bacteria Mycobacterium tuberculosis and Listeria sp.; and by the protozoan parasites Toxoplasma and Trypanosoma. Induction of protective CTL responses against influenza A virus has been demonstrated in FIG. 14. Furthermore, the immunisation regime described herein is expected to be of value in immunising against forms of cancer where CD8+ T cell responses plays a protective role. The induction of protective CTL responses using the DNA prime MVA boost regime against tumours is shown in FIG. 13. Specific examples in humans include melanoma, cancer of the breast and cancer of the colon.

### Example 7

### Immunogenicity and Protective Efficacy Against Tuberculosis

### Example 7A

### ESAT6 and MPT63 Antigens

Since secreted antigens that are released from live mycobacteria are thought to be important in the generation of protective immunity, the present inventors selected two secreted antigens from M. tuberculosis for inclusion in the vaccines. The first, ESAT6 (early secreted antigenic target 6), is relatively specific for M. tuberculosis, and importantly, is not present in M. bovis BCG (Harboe, M., et al., Infect.Immun. (1996) 64: 16-22). ESAT 6 is a key antigenic target early in murine infection (Brandt, L., et al., J.Immunol. (1996) 157: 3527-3533) and is a human CTL target (Lalvani, A., et al., Proc.Natl.Acad.Sci. U.S.A. (1998) 95: 270-275). The second antigen, MPT63 (mycobacterial protein tuberculosis 63), is present in some strains of M. bovis BCG (Manca, C., et al., Infect.Immun. (1997) 65: 16-23)). A polyprotein DNA construct and recombinant MVA virus containing both antigens were generated and we assessed the immunogenicity of these constructs, individually and in combination. The most immunogenic vaccine combinations were then assessed in murine challenge experiments with M. tuberculosis.

Construction of Plasmid DNA and Recombinant MVA Tuberculosis Vaccines

A single coding sequence containing the TPA leader sequence, ESAT6 and MPT63 genes and the Pk antibody epitope (TEMPk) was constructed and ligated into the plasmid vector pSG2, creating the DNA vaccine pSG2.TEMPk. Expression of the fusion protein was shown to be in the cytoplasm.

The recombinant MVA was purified from a transfection of wild type MVA and a vaccinia shuttle vector containing the sequence TEMPk.

DNA and MVA vaccines both induce peptide-specific IFNγ producing CD4+ T cells. C57B1/6 mice were immunised with DNA(i.m), MVA(i.d.) or a combination of the two. Using overlapping peptides which span the length of both antigens and an IFN-γ ELISPOT assay, we identified responses to several peptides in the splenocytes of immunised mice. Responses were seen to two peptides within ESAT 6 (E1 and E2) and four peptides within MPT63 (M3,15,27 and 28) (Table 13). To assess the phenotype of the cells responding to these peptides, CD4+ and CD8+ cells were depleted using magnetic beads. The responses to all six peptides were fully abrogated when CD4+ T cells were depleted, and unaffected when CD8+ T cells were depleted (Table 13). Assays were performed both ex-vivo (peptides E1 and E2) and after culturing the cells with peptide for 5-7 days (all peptides, see methods).

TABLE 13

Peptides showing T cell epitopes identified in
ESAT 6 and MPT63

| | | | | |
|---|---|---|---|---|
| Antigen | Peptide | Sequence | CD4 | CD8 |

| | | | | |
|---|---|---|---|---|
| ESAT6 | 1 | MTEQQ WNFAG IEAAA | + | − |
| | | (SEQ ID NO: 79) | | |
| | 2 | WNFAG IEAAA SAIQG | + | − |
| | | (SEQ ID NO: 80) | | |
| MPT63 | 3 | VAVVA MAAIA TFAAP | + | − |
| | | (SEQ ID NO: 81) | | |
| | 15 | VAGQV WEATA TVNAI | + | − |
| | | (SEQ ID NO: 82) | | |
| | 27 | GKIYF DVTGP SPTIV | + | − |
| | | (SEQ ID NO: 83) | | |
| | 28 | DVTGP SPTIV AMNGM | + | − |
| | | SEQ ID NO: 84) | | |

Homologous Boosting of DNA and MVA Induced Responses

The highest frequency of IFN-γ secreting T cells (SFC) was to the first ESAT 6 peptide, E1 (Table 14). A single dose of DNA failed to generate any detectable responses, but when repeated twice, or three times at two weekly intervals, consistent responses were seen. After three immunisations, the number of SFC was more than double that seen after two immunisations; the mean response to E1 after two doses was 30 SFC per 10⁶ splenocytes, and after three doses was 75 SFC. A single immunisation with MVA generated responses with a mean frequency of spot forming cells to E1 of 20 per million, which were modestly improved following a second dose of MVA (mean frequency of SFC to E1=30 ). Thus gamma-interferon secreting CD4 T cell responses are induced to an encoded CD4 T cell epitope by immunisation with this replication-impaired poxvirus.

TABLE 14
Summary of peptide specific T cell responses to the two constructs.

| | SFC to individual peptides | | | | | |
|---|---|---|---|---|---|---|
| Condition | E1 | E2 | M3 | M15 | M27 | M28 |
| DNA × 1 | — | — | — | — | — | — |
| DNA × 2 | 30 | 5 | — | 5 | 5 | 5 |
| DNA × 3 | 75 | 50 | — | 15 | 40 | 45 |
| MVA × 1 | 20 | 10 | 5 | — | 5 | 5 |
| MVA × 2 | 30 | 10 | 5 | — | 10 | 10 |
| DNA/MVA | 130 | 26 | 12 | 17 | 5 | 10 |
| MVA/DNA | 130 | 70 | 10 | 10 | 10 | 5 |
| DNA × 3/MVA | 360 | 250 | 25 | 100 | 30 | 50 |

Numbers represent mean of 3-10 mice per 10⁶ splenocytes. Standard error is <20%.

Heterologous Prime-Boost Regimes Increased the Magnitude of the Observed CD4 T Lymphocyte Responses

Having detected these responses using the vaccines individually, the role of heterologous prime-boost regimes, i.e. using either the DNA or MVA construct to prime the response and the second construct to boost two weeks later, was assessed. Heterologous boosting, either DM or MD, produced stronger responses than homologous boosting of either DD or MM (Table 14). The mean response to peptide E1 was increased by more than 4-fold to 130 SFC and, surprisingly—in view of the finding on induced CD8 T cell responses (Schneider, J., et al., Nat.Med. (1998) 4: 397-402), this occurred regardless of which order the two vaccines were given. The response to peptide E2 was slightly stronger when MVA was followed by DNA rather than the reverse order. The responses to peptides M3 and M15 were stronger with a heterologous boost, regardless of the order in which the vaccines were given, whilst the responses to peptides M27 and M28 were weak and not boosted. The strongest response was seen when DNA was given three times and then boosted with MVA once (DDDM). In this case the mean response to E1 was increased almost 5-fold from 75SFC to 360SFC. The responses to the other peptides were also higher after DDDM compared with DM.

In further studies using plasmid DNAs expressing the M. tuberculosis antigen, Ag85A and a recombinant MVA expressing the same antigen the induction of CD4 T cells to a CD4 T cell epitope was observed in Balb/c mice. Depletion studies using antibody coated beads confirmed that the response to the P15 peptide in this antigen was CD4-dependent. In the same experiment CD8 T cells were also induced to the P15 CTL epitope in Ag85 by both DNA immunisation and by recombinant MVA immunisation. Stronger response to both the CD4 and CD8 epitope were observed after prime-boost immunisation, priming with DNA and boosting with MVA. Thus both recombinant MVA immunisation and heterologous prime-boost immunisation can generate CD4 and CD8 gamma-interferon-secreting T cell responses to epitopes in the same antigen.

Challenge Experiments

Heterologous prime-boost regimes generated the highest levels of IFN-γ secreting CD4+ T cells, and therefore the protective efficacy of these regimes was assessed in challenge experiments using the ESAT-MPT63 constructs. The first challenge experiment compared the protective efficacy of DNA prime/MVA boost (DM), with MVA prime/DNA boost (MD) supplemented in each case with a second MVA boost. The second challenge experiment assessed the protection conferred by three sequential immunisations with DNA followed by a single MVA immunisation (DDDM). In both experiments, BCG was used as a positive control. The immunogenicity of each vaccine regime was assessed in 2-3 mice before the remainder of the group were challenged. In the first experiment, the immune responses were not as strong as previously seen (average response to E1 25 SFC). Therefore in this case a second dose of MVA was administered to both groups prior to challenge. In the second challenge experiment, the average response to the dominant peptide, E1, was 225 SFC. The challenge was conducted two weeks after the last subunit vaccine immunisation, shortly after the T cell response had reached a plateau (unpublished data).

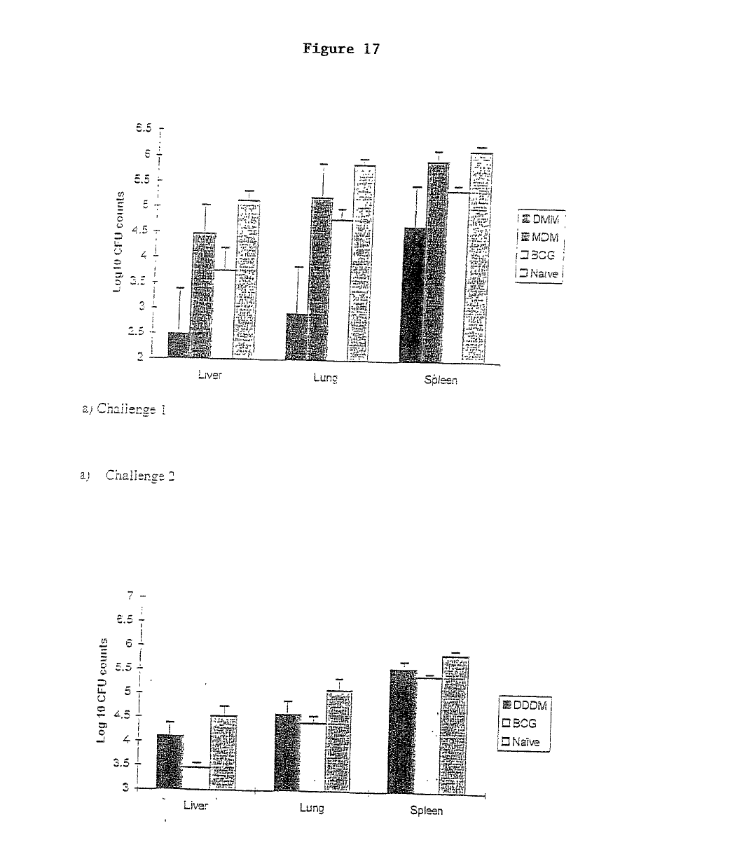

To assess the efficacy of the immunisation regimes at 8 weeks, organs from all mice remaining at 8 weeks were harvested, and CFU counts determined. In both challenge experiments, as expected, the CFU counts in the BCG group were significantly lower than in the naive group in all three organs (p<0.05, FIG. 17). In the first challenge experiment, the CFU counts in all three organs in the DMM group were significantly lower than the naive group (p<0.05). The CFU counts in the MDM group in all three organs were not significantly different from the naive control group. However, in the second challenge experiment, the lung was the only organ in which the CFU counts in the DDDM group were significantly lower than the naive control group (p<0.05). The liver and spleen counts were not significantly different between these two groups. The DMM/MDM/DDDM group CFU counts were not significantly different from the BCG group in any organ, in either experiment.

These results demonstrate the immunogenicity and protective efficacy against M. tuberculosis of a MVA and DNA vaccine vectors that induce gamma-interferon secreting CD4 T lymphocyte responses and also of heterologous prime-boost immunisation regimes using these vaccines. DNA vaccination is known to induce a T_{H}1 type immune response, and therefore we chose the quantification of a T_{H}1 cytokine, IFN-γ, in an ELISPOT assay as our functional outcome measure. This assay is a very sensitive method of quantifying T cell function (Lalvani, A., et al., J.Exp.Med. (1997) 186: 859-865). Proliferation assays are an alternative measure of CD4+ T cell response, but this is not a readout of an effector response and importantly gamma-interferon secretion and proliferation responses are often negatively correlated (Troye-Blomberg et al., Flanagan et al 2000). There are two reasons why measuring IFN-γ production is a more relevant outcome measure in an M. tuberculosis challenge model. IFN-γ is an essential component of the protective immune response to tuberculosis, as IFN-γ knockout mice are much more susceptible to challenge with M. tuberculosis than their wild type counterparts (Cooper, A. M., et al., J.Exp.Med. (1993)178: 2243-2247). In addition, a mutation in the human IFN-γ receptor gene confers susceptibility to atypical mycobacterial infection (Newport, M. J., et al., N.Engl.J.Med. (1996) 335: 1941-1949.).

The recombinant MVA as well as the DNA vaccine each individually generated specific IFN-γ secreting CD4+ T cells to the same peptides. There were no IFN-γ secreting CD8+ T cell responses observed to these constructs, presumably as a results of the absence of a peptide with high binding affinity for the relevant MHC class I molecules in this strain of mice (C57/BL6). As the peptides used to assess the responses spanned the length of both antigens this effectively excludes the presence of a CD8 epitope for this mouse strain. These constructs therefore allowed us to assess the effect of each vaccine type and of prime-boost regimes on CD4+ T cell responses. Although each vaccine type clearly induced CD4 T cell responses heterologous prime-boost regimes with the two vaccines generated stronger CD4+ T cell responses than homologous boosting. Interestingly, the order in which the two vaccines were given made no clear difference to the strength of the immune responses generated. Priming with DNA and boosting with MVA, or priming with MVA and boosting with DNA both produced a 3-4 fold increase in the number of IFN-γ secreting CD4+ T cells specific for the first ESAT 6 peptide. This contrasts with published work on CD8+ T cell responses, where DNA prime followed by MVA boost is the only order in which high levels of immunogenicity and protection are seen (Schneider, J., et al., Nat.Med. (1998) 4: 397-402).

At eight weeks, levels of protection with DNA/MVA immunisation regimes were equivalent to those obtained with BCG and the protection in the BCG immunised group is in the same order as that previously published (Tascon, R. E., et al., Nat.Med. (1996) 2: 888-892).

In the first challenge experiment, the group immunised with DNA/MVA showed levels of protection equivalent to BCG in all three organs. In the second experiment, protection in the DNA/MVA immunised group was only seen in the lungs at eight weeks. Previous authors have observed varying protective effects in different organs depending on the time from challenge to harvesting. Zhu et al. reported protection after DNA immunisation in the lungs four weeks after challenge, but only observed protection in the lungs and spleen 12 weeks after challenge (Zhu, X., et al., J.Immunol. (1997) 158: 5921-5926). As the primary route of infection in humans is the pulmonary route, the lung is the most relevant organ in which to identify protection. More relevant aerosol models of M. tuberculosis challenge have been developed, and it will be important to see whether vaccines that confer protection against a systemic route of challenge remain protective against an aerosol challenge, and whether protection in the lungs is maintained.

DNA priming seemed to be necessary for protection to occur in the challenge experiments as in the first challenge experiment, protection was seen in the DMM group but not the MDM group. Note that the lack of protection in the MDM group at 24 hours and at eight weeks effectively rules out a non-specific protective effect of the subunit vaccines administered up to two weeks before challenge. It is uncertain why protection was achieved in the DMM but not the MDM groups when the immunisation order (Table 14) appeared not to affect immunogenicity. The difference however, may relate to the timing of the second MVA boost, as the two MVA doses were given a month apart in the M/D/M group. It may be that within this interval an antibody response to the MVA abrogated the boosting effect.

The mechanism by which the response to a DNA priming vaccination can be boosted by a subsequent immunisation with a recombinant virus encoding the same antigen has not been fully elucidated. Without wishing to be bound by theory, the present inventors predict that it may relate to the induction by DNA of memory T cells to an immunodominant epitope(s), that expand rapidly on exposure to a recombinant virus carrying the same epitope (Schneider, J., et al., Immunological Reviews (1999) 170: 29-38). It is possible that the mechanisms involved in the boosting of CD8+ T cells are different to those involved in the boosting of CD4+ T cells.

### Example 7B

### Antigen 85A

DNA and MVA constructs expressing antigen 85A were used to immunise two strains of mice: BALB/c and C57BL/6. Several peptide responses were detected in the splenocytes from immunised mice using the IFN-γ Elispot assay and the overlapping peptides spanning the length of antigen 85A. Mice were immunised with DNA and/or MVA, alone and in combination, in order to determine the optimal immunisation regimens.

All the strongest responses identified were in BALB/c mice. All further work with these constructs was restricted to this mouse strain. The DNA and MVA constructs induced responses to the same peptides. Responses to four of the overlapping peptides were identified using the splenocytes of immunised mice; p11, p15, p24 and p27. The strongest responses were to peptides p11 and p15.

Depletion Studies

To assess the phenotype of the T cell responses to these peptides, CD4+ and CD8+ cells were depleted using magnetic beads. The response to p11 was almost completely abrogated by CD8+ T cell depletion. The response to p15 was completely abrogated by CD4+ T cell depletion. The weaker responses to peptides 24 and p27 were both completely abrogated by CD4+ T cell depletion. These results are summarised in Table 15.

In summary, immunisation with the DNA and MVA constructs expressing antigen 85A induced an immunodominant CD8+ epitope (p11), an immunodominant CD4+ epitope (p15) and 2 weaker CD4+ epitopes (p24 and p27).

TABLE 15

Depletion studies on responses identified within
antigen 85A.

| | | | | |
|---|---|---|---|---|
| | | Un- | CD4⁺ | CD8⁺ |
| | | depleted | De- | De- |
| Pep- | | response | pletion | pletion |
| tide | Sequence | (SFC) | (SFC) | (SFC) |

| | | | | |
|---|---|---|---|---|
| P11 | EWYDQSGLSVVMPVGGQSSF | 250 | 200 | 10 |
| | (SEQ ID NO: 85) | | | |
| P15 | TFLTSELPGWLQANRHVKPT | 300 | 0 | 250 |
| | (SEQ ID NO: 86) | | | |
| P24 | QRNDPLLNVGKLIANNTRVW | 30 | 0 | 20 |
| | (SEQ ID NO: 87) | | | |
| P27 | LGGNNLPAKFLEGFVRTSNI | 30 | 0 | 25 |
| | (SEQ ID NO: 88) | | | |

The responses generated by a single immunisation with either construct were weak and only slightly increased by homologous boosting with the same construct. Heterologous boosting of DNA with MVA produced significantly higher frequencies of both the CD4+ (p15) and CD8+ (p11) T cells. Heterologous boosting of MVA with DNA boosted the frequency of CD4+ T cells (15) but did not increase the CD8+ (p11) T cell response. Three DNA immunisations followed by a single MVA boost (DDDM) generated the highest frequency of IFN-γ secreting T cells. This is consistent with the results using the ESAT6/MPT63-expressing constructs, where the most immunogenic regime was DDDMM.

These results are summarised in table 16.

TABLE 16
Summary of peptide specific T cell responses
to the antigen 85A constructs.

| | SFC per 10⁶ splenocytes to individual peptides^{a} | | | |
|---|---|---|---|---|
| Immunisation | Peptide number | | | |
| regime | 11 | 15 | 24 | 27 |
| DNA × 1 | 29 | 24 | 32 | 19 |
| DNA × 2 | 11 | 14 | 10 | 16 |
| DNA × 3 | 70 | 31 | 15 | 16 |
| MVA × 2 | 9 | 12 | 10 | 6 |
| DNA/MVA | 80 | 77 | 9 | 32 |
| MVA/DNA | 8 | 103 | 9 | 10 |
| DNA × 3/MVA | 312 | 325 | 14 | 141 |

^{a}Numbers represent means of SEC per 10⁶ splenocytes for 3-10 mice per group. Standard error is <20%

Challenge Experiments

Once optimal immunisation regimes with the antigen 85A constructs had been determined, the protective efficacy of these regimes was evaluated in challenge studies with M.tb. Initially, 1×10⁶ cfu M.tb was used as a challenge dose, half a log lower than the challenge dose used for the C57BL/6 mice. This was because the literature suggests that BALB/c mice are slightly more susceptible to challenge with M.tb than C57BL/6 mice. Eight weeks was chosen as a time point for harvest, to enable comparison with the challenge results using the ESAT6/MPT63 constructs. Previous authors have shown a protective effect in BALB/c mice at 6 weeks after an i.p challenge with M.tb.

Comparison of Heterologous vs Homologous Prime-Boost Regimes

A challenge experiment was set up to compare the protective efficacy of heterologous and homologous prime-boost regimes using the antigen 85A expressing constructs. The immunogenicity results using these constructs had confirmed that heterologous boosting produced higher levels of specific CD4+ and CD8+ T cells than homologous boosting. A control group of mice that received 3 doses of antigen 85A DNA followed by a single dose of non-recombinant MVA were included to assess the specificity of the boosting effect of MVA.

There were 5 groups in this experiment:

(ii) Naïve

(iii) BCG

(iv) DDD

(v) DDDM

(vi) DDD(Non-recombinant MVA[NRM])

There were 10 mice in all groups except the DDDM group, which had 7 mice. Two to three mice were harvested from each group for immunogenicity at the time of challenge. BCG was given at the same time-point as the first DNA immunisation. Mice were challenged with 10⁶cfu M.tb i.p., 2 weeks after the final immunisation, and harvested 8 weeks after challenge.

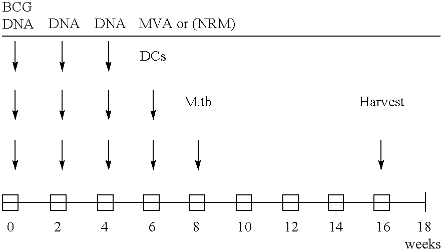

The Elispot results for the DDDM group showed high levels of T cell responses consistent with the previous immunogenicity results for this regime. The results for the DDD and DDD(NRM) groups showed low level responses. There was no boosting effect seen with non-recombinant MVA. These results are summarised in Table 17.

TABLE 17
Challenge 4: Mean SFC per 10⁶ splenocytes
for heterologous and homologous immunisation regimes

| | P11 | P15 | P24 | P27 |
|---|---|---|---|---|
| DDD | 9 | 0 | 0 | 0 |
| DDD (NRM) | 0 | 8 | 0 | 7 |
| DDDM | 217 | 245 | 0 | 217 |

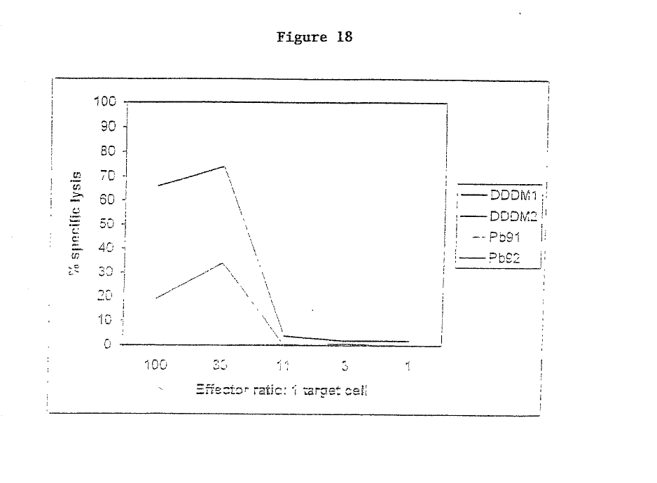

A ⁵¹Cr release assay was performed on the splenocytes from mice in the DDDM group. The results of this assay showed that in one of the two mice harvested in the DDDM group, high levels of specific lysis (60-70%) could be demonstrated. In the other mouse, the level of lysis was much lower (20-30%). These results are summarised in FIG. 18.

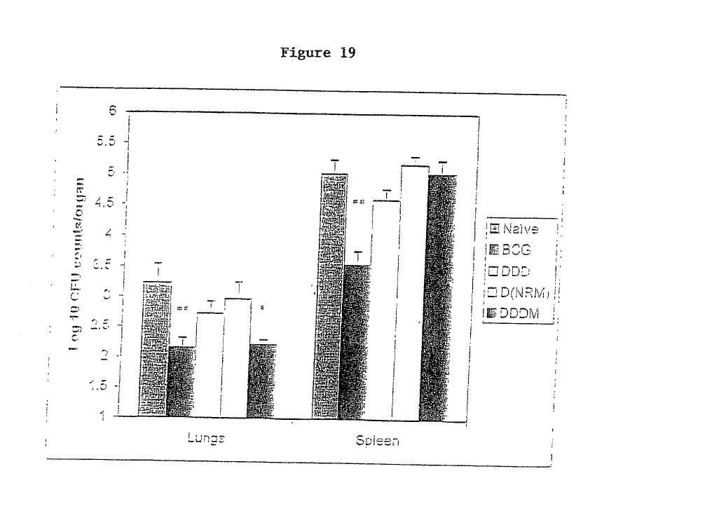

The heterologous prime-boost regime (DDDM) confers protection in the lungs equivalent to BCG when compared to the naïve control group (p=0.010). No protection is seen in the spleen in the DDDM group. The homologous regime, DDD, both alone and boosted with non-recombinant MVA, DDD(NRM), did not confer any significant protection against challenge. As expected, there is a significant protective effect of BCG in both the lungs and spleen, when this group is compared to the naïve control group (lungs: p=0.009; spleen: p<0.001). These results are summarised in FIG. 19.

Discussion

The DNA vaccine and recombinant MVA expressing antigen 85A generated specific IFN-γ secreting CD4+ and CD8+ T cells to the same four peptides. Heterologous prime-boost regimes with the two vaccines generated higher frequencies of T cell responses than homologous boosting. CD4+ T cell responses were increased regardless of the order of immunisation. This is consistent with the results obtained with the ESAT6/MPT63 expressing constructs (Example 9A). The CD8+ T cell response induced by the antigen 85A expressing constructs was only boosted with the DNA prime-MVA boost immunisation regime and not when the constructs were given in the opposite order. This is consistent with previously published work on boosting CD8+ T cell responses.

The processing pathways for the induction of class-I restricted CD8+ T cell responses and class-II restricted CD4+ T cell responses are different. This may explain why DNA immunisation boosts CD4+, but not CD8+ T cell responses. DNA vaccination is known to be good at priming class-I restricted CD8+ T cells, as the endogenously produced antigen can access the class I pathway. Recombinant viral vectors probably also use the endogenous class I pathway. It may be that the MVA immunisation induces different cytokines to the DNA immunisation. One possible cytokine is IL4. The boosting effect of MVA can be abrogated by the co-administration of IL4 antibodies with the MVA (Sheu, personal communication). IL4 may be necessary to boost a memory CD8+ response but not a CD4+ response.

In the challenge experiment, the enhanced CD4+ and CD8+ T cell responses induced with the heterologous prime-boost immunisation regimes conferred protection in the lungs equivalent to, but not greater than BCG.

### Example 8

### Induction of γ-IFN Secreting CD4 T Cell Responses to Candidate Malaria Vaccines in Mice and Humans

A polyepitope string of mainly malaria (P. falciparum) CD8 T cell peptide epitopes has been described previously. This construct also expresses CD4 T cell epitopes from tetanus toxoid and from the 38 Kd mycobacterial antigen of various strains of M. tuberculosis and M. bovis (labelled BCG in Gilbert S C, et al., Nat Biotechnol. (1997) November;15(12):1280-4). The DNA encoding this polyepitope string has been ligated to DNA encoding the entire coding sequence of the P. falciparum (strain T9/96) thrombospondin-related adhesion protein (TRAP) antigen. This so-called ME-TRAP (multi-epitope-TRAP) insert has been cloned into a plasmid DNA expression vector and into MVA. These constructs were immunogenic for the induction of gamma-interferon-secreting T cells in Balb/c and C57/BL6 strains of mice. These latter candidate DNA and MVA malaria vaccines have been manufactured according to GMP guidelines.

Healthy volunteers were immunised with three vaccinations consisting of either the plasmid DNA intramuscularly (imDNA) at 0.5 mg or 1 mg or by gene gun (ggDNA) at 4 μg, or the recombinant MVA (5×10⁷ plaque forming units (pfu) intradermally). Four vaccination regimes were used:

1. 1 vaccination with imDNA followed by two with MVA (DM2) (n=3).

2. 2 vaccinations with imDNA followed by one with MVA (D2M) (n=3).

3. 3 vaccinations with imDNA followed by one with MVA (D3M) (n=6).

4. 2 vaccinations with ggDNA followed by one with MVA (G2M) (n=6).

5. 3 vaccinations with ggDNA followed by one with MVA (G3M) (n=2).

6. 3 vaccinations with MVA (M3) (n=10).

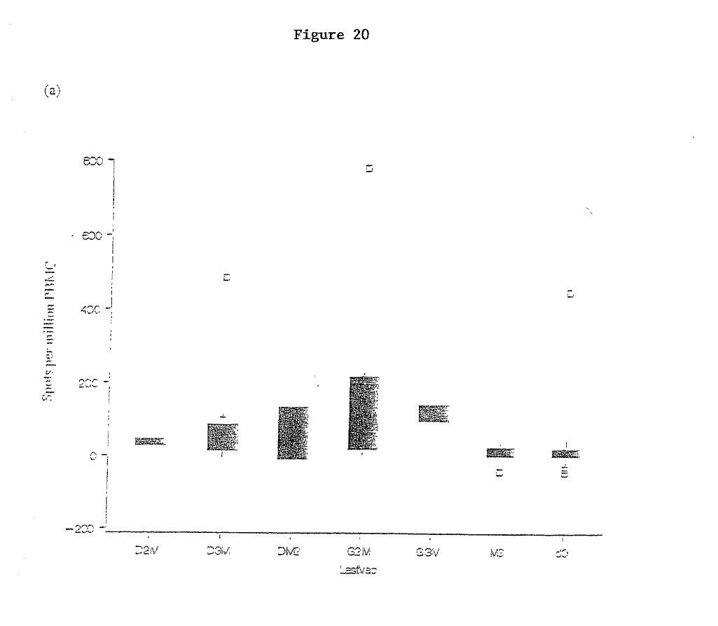

Also shown in FIG. 20 are the prevaccination (d0) responses (n=30).

Peripheral blood mononuclear cells (PBMC) were assayed using INF-ELISPOT 7 days after the last immunisation for their responses to a pool of peptides spanning the first 110 amino acids of TRAP from P. falciparum strain T9/96 (TT1-10—Sequences shown in Table 18). Heterologous vaccination of G2M induced the strongest responses to this set of peptides, significantly higher than the responses induced by the homologous vaccination (M3) alone (FIG. 20—P=0.0172, Mann-Whitney test) or the pre-vaccination (d0) samples (P=0.0235). Overall, the responses in the prime-boosted volunteers were significantly higher than in the homologous vaccinated volunteers (P=0074), indicating that heterologous prime-boost vaccination induces responses to this pool of peptides.

TABLE 18

Sequences of the peptides in the TT1-10 pool:

| | |
|---|---|
| Pep no. | Sequence |

| | |
|---|---|
| 1 | MMHLGNVKYLVIVFLIFFDL |
| | (SEQ ID NO: 89) |
| 2 | VIVFLIFFDLFLVNGRDVQN |
| | (SEQ ID NO: 90) |
| 3 | FLVNGRDVQNNIVDEIKYSE |
| | (SEQ ID NO: 91) |
| 4 | NIVDEIKYSEEVCNDQVDLY |
| | (SEQ ID NO: 92) |
| 5 | EVCNDQVDLYLLMDCSGSIR |
| | (SEQ ID NO: 93) |
| 6 | LLMDCSGSIRRHNWVNHAVP |
| | (SEQ ID NO: 94) |
| 7 | RHNWVNHAVPLAMKLIQQLN |
| | (SEQ ID NO: 95) |
| 8 | LAMKLIQQLNLNDNAIHLYV |
| | (SEQ ID NO: 96) |
| 9 | LNDNAIHLYVNVFSNNAKEI |
| | (SEQ ID NO: 97) |
| 10 | NVFSNNAKEIIRLHSDASKN |
| | (SEQ ID NO: 98) |

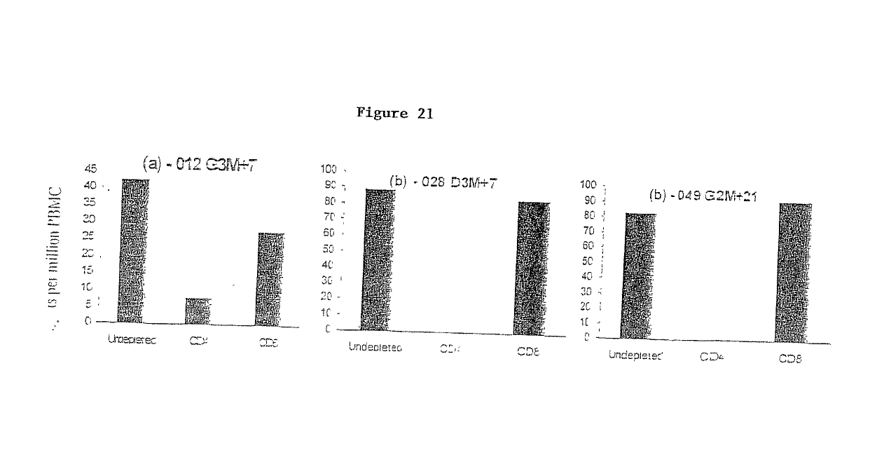

Responses to the TT1-10 pool from the TRAP antigen that were induced by vaccination were shown to be dependent on CD4⁺ T cells and not on CD8⁺ T cells in all three volunteers tested (FIG. 21). PBMC from three volunteers were frozen either 7 days after vaccination (donors 012 and028) or 21 days after vaccination (donor 049); these were thawed and tested against pool TT1-10, after removing either CD4⁺ T cells or CD8⁺ T cells using the Dynal Dynabead system. The responses to this pool in all three cases were dependent on CD4⁺ cells, but not CD8⁺ cells (FIG. 21).

Thus for the TRAP pool TT1-10, heterologous prime-boost vaccination induces responses that are significantly higher than homologous vaccination, and these responses are dependent on CD4⁺ T cells.

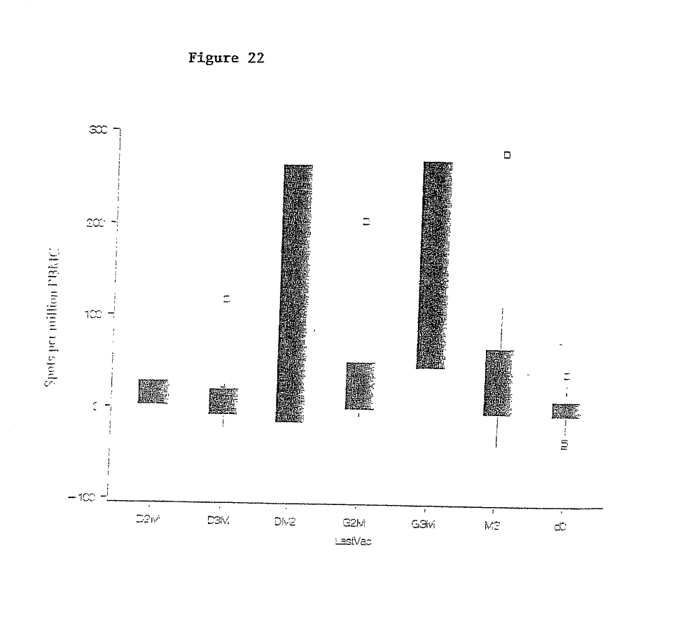

In the volunteers shown in FIG. 20, responses were also induced by heterologous prime-boost vaccination to the well characterised CD4+ T cell tetanus toxoid epitope (FTTp—sequence QFIKANSKFIGITE) (SEQ ID NO: 99) (FIG. 22). While none of the individual groups were significantly above the d0 responses by a Mann-Whitney test, pooling the results from all groups showed that volunteers that received heterologous prime-boost vaccinations showed significantly induced responses to this CD4⁺ T cell epitope (P=0.0064). As expected based on the preclinical data shown above responses in prime-boost vaccinated volunteers were higher than the responses in homologous vaccinated volunteers (FIG. 22).

Thus recombinant MVA induces IFNγ secreting CD4⁺ T cells in humans, and does so more efficiently in a heterologous prime-boost vaccination strategy than in a homologous vaccination strategy.

The capacity of recombinant replication-impaired poxvirus vectors to induce such functional CD4 T cell responses both used alone and in prime-boost combinations, both in animals and in man, will have widespread utility both for prophylactic and for therapeutic vaccination. Such application include but are not limited to prophylactic vaccination against tuberculosis, HIV, malaria. H. pylori, influenza, hepatitis, CMV, herpes virus-induced diseases and other viral infections, leprosy, non-malarial protozoan parasites such as toxoplasma, and various malignancies, and to therapeutic vaccination against tuberculosis, persistent viral infections such as HIV and chronic hepatitis B and C and many malignancies.

Materials and Methods

M. tuberculosis Stocks

M. tuberculosis (H37Rv) was grown in Dubos medium and incubated at 37° C. for 21-28 days. The solution was centrifuged, resuspended in TSB/glycerol and stored at −70° C. after titration. Stock solutions were sonicated before use.

Plasmid DNA Constructs

M. tuberculosis (H37Rv) was heat inactivated and DNA extracted (QIAamp,Qiagen, Hilden, Germany). Oligonucleotide primers (Genosys Biotechnologies Ltd, Pampisford, Cambs) were used to amplify the ESAT6 and MPT63 gene. The PCR products were extracted from agarose gel and purified (QIAquick kit, Qiagen). The tissue plasminogen activator (TPA) leader sequence was also amplified. The three PCR products were sequenced, then ligated together to form a single coding sequence with the Pk antibody epitope at the 3′ end (TEMPk). The TEMPk fragment was ligated into the plasmid vector pSG2, creating pSG2.TEMPk. This plasmid has the CMV promoter with intron A, the bovine growth hormone poly A sequence, and the kanamycin resistance gene as a selectable marker. Expression of the TEMPk fusion protein in COS-1 cells was detected by immunofluorescence using antibodies to the Pk tag (Serotech, UK) followed by fluoroscein isothiocyanate isomer (FITC) labelled secondary antibodies (Sigma). Nuclear staining showed the protein to be in the cytoplasm. Plasmid DNA for injections was purified using anion exchange chromatography (Qiagen) and diluted in endotoxin free phosphate buffered saline (Sigma).

Construction of Recombinant Modified Vaccinia Ankara (MVA)

The DNA sequence TEMPk was cloned into the Vaccinia shuttle vector pSC11. BHK cells were infected with wild type MVA (A Mayr, Veterinary Faculty, University of Munich, Germany) at a multiplicity of infection of 0.05, then transfected with the recombinant shuttle vector. Recombinant virus was selected for with bromodeoxyuridine and then plaque purified on CEF cells.

Animals and Immunisations

Female C57/BL6 mice aged 4-6 weeks (Harlan Orlac, Shaws Farm, Blackthorn, UK) were injected with plasmid DNA (25 μg/muscle) into both tibialis muscles, under anaesthesia. Recombinant MVA (10⁶ pfu) was injected intradermally. Mice were immunised at two week intervals and harvested for immunogenicity two weeks after the last immunisation. For the challenge experiments mice were infected two weeks after the last immunisation. A BCG control group was immunised with 4×10⁵ cfu M. bovis BCG (Glaxo) intradermally at the time of the first DNA/MVA immunisation.

Preparation of Splenocytes

Mice were sacrificed and spleens removed using aseptic technique. Spleens were crushed and the resulting single cell suspension filtered through a strainer (Falcon, 70 μm, Becton Dickson, N.J.). Cells were pelleted and the red blood cells lysed using a hypotonic lysis buffer. Cells were then washed and counted. Splenocytes were resuspended in alpha-MEM medium with 10% FCS, 2 mM glutamine, 50 U/ml penicillin, 50 mg/ml streptomycin, 50 μM 2-mercaptoethanol and 10 mM Hepes pH 7.2 (all from Gibco).

Peptides

Overlapping peptides spanning the length of both antigens were purchased from Research Genetics (Huntsville, Ala., USA). The peptides were 15 amino acids in length and overlapped by 10 amino acids (Table 18).

ELISPOT Assays

The number of IFN-γ secreting peptide-specific T cells was determined using the overlapping peptides in an ELISPOT assay (Schneider, J., et al., Nat.Med. (1998) 4: 397-402). Briefly, 96 well nitro-cellulose plates (Milliscreen MAHA, Millipore, Bedford, Mass.) were coated with 15 μg/ml of the anti-mouse IFN-γ monoclonal antibody R4-6A2 (hybridoma purchased from the European Collection of Animal Cell Cultures). After incubating at 4° C. overnight, the wells were washed with PBS and blocked with 100 μl RPMI:10% FCS for one hour at room temperature. Splenocytes were added to the wells (10⁶ cells/well) with peptide (final concentration 2 μg/ml). Conconavalin A (Sigma-Aldrich Co Ltd, Poole, UK) was used as a positive control for the assay. Control wells had no peptide. After incubating the plate overnight at 37° C. with 5% CO₂ in air, it was developed as previously described (Schneider, J., et al., Nat.Med. (1998)4:397-402). The spots were counted using a dissecting microscope. Numbers refer to spot forming cells per million effector cells (SFC).

Cell Depletions

CD4+ and CD8+ T cell depletions were performed using anti-CD4 or anti-CD8 monoclonal antibodies conjugated to ferrous beads (Dynal, Oslo). Splenocytes from immunised mice were restimulated in six well tissue culture plates with 1 μg/ml of the relevant peptide, and on day 3 of culture 10 U/ml of human IL2 (Lymphocult-T, Biotest, Dreieich, Germany) was added. At days 5-7 the restimulated splenocytes were washed twice and incubated on ice for 30 minutes with one of the 2 antibodies (bead:cell=5:1). An ELISPOT assay was then performed as before, using the depleted cell populations. Assays for peptides E1 and E2 were also performed ex-vivo. Depletion studies for each peptide response were performed twice.

Challenge Experiments

Mice were infected with 5×10⁶ cfu M. tuberculosis (H37Rv) by intraperitoneal injection, in a Category III isolator unit. To assess the baseline level of infection, the liver, lungs and spleen from 2-5 mice from each group were harvested and weighed, twenty-four hours after infection. The organs from the remaining 7-10 mice in each group were harvested eight weeks after challenge. Organs were homogenised by vortexing with 5 mm glass beads in 1 ml of sterile PBS and serial dilutions were plated onto Middlebrook plates. Plates were incubated for 21 days at 37° C. and colony counts/gram tissue were then calculated. The Mann-Whitney U test was used to compare CFU counts between groups.

Cell Preparation

Peripheral blood mononuclear cells (PBMC) were prepared from peripheral blood by Ficoll separation. Assays were either performed on fresh blood, or frozen in 10% DMSO/90% FCS before being assayed, as detailed in the text. All culture medium was supplemented with 10% human AB serum, 2 mM Glutamine and 100 U ml-1 Penicillin/Streptomycin. Cells were depleted using the Dynal Dynabead system at 5-10 beads/cell.

Ex Vivo ELISPOT Assays

The culture medium was RPMI 1640. ELISPOTs were performed on Millipore MAIP S45 plates with MabTech antibodies according to the manufacturer's instructions: 4×105 PBMC were incubated for 18-20 h on the ELISPOT plates in the presence of peptides each at 25 μg ml-1. The plates were then washed in Phosphate Buffered Saline (PBS) containing 0.5% Tween-20 (PBST), and a biotinylated anti-IFNγ antibody diluted in PBS was added, and incubated for 2-24 h, the plates were then washed in PBST, and streptavidin alkaline phosphatase diluted 1:1000 in PBS was added. After 1-2 h at room temperature, the plates were washed and developed using the BioRad precipitating substrate kit. Plates were counted by the AutoImmun Diagnostika system. Results are expressed as spot forming units (sfu) per million cells added to the well and are calculated as the difference between the test and the response to medium alone.

### Example 9

### Induction of Melanoma-Specific CD8+ T Cells in Melanoma Patients Immunised with Recombinant DNA and MVA

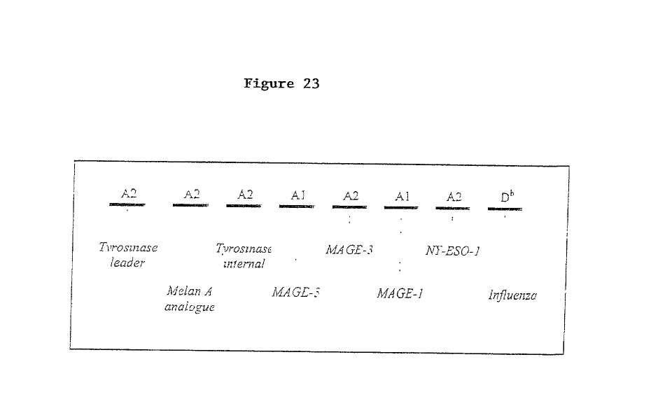

There is evidence that a CD8+ T cell response can control tumours in melanoma patients. In order to treat melanoma patients by inducing melanoma-reactive CD8+ T cells using the invention described, a polyepitope string (Mel3) encoding a series of seven known cytotoxic T lymphocyte (CTL) epitopes from five human melanoma antigens (tyrosinase, Melan A, MAGE-1, MAGE-3 and NY-ESO-1) was constructed and inserted into plasmid DNA (pSG2) and MVA. The recombinant constructs are termed pSG2.Me13 and MVA.Me13. A schematic diagram of the melanoma poly-epitope gene is shown in FIG. 23. The individual CD8+ T cell epitopes are presented by two common human leukocyte antigen (HLA) types (HLA-A1 and HLA-A2) and are included to ensure an immune response to the vaccine in a significant proportion of the population. As the human melanoma CTL epitopes are not recognised in mice, a CTL epitope from the influenza nucleoprotein was included in the polyepitope gene to enable the immunogenicity of the plasmid to be assessed in preclinical murine models.

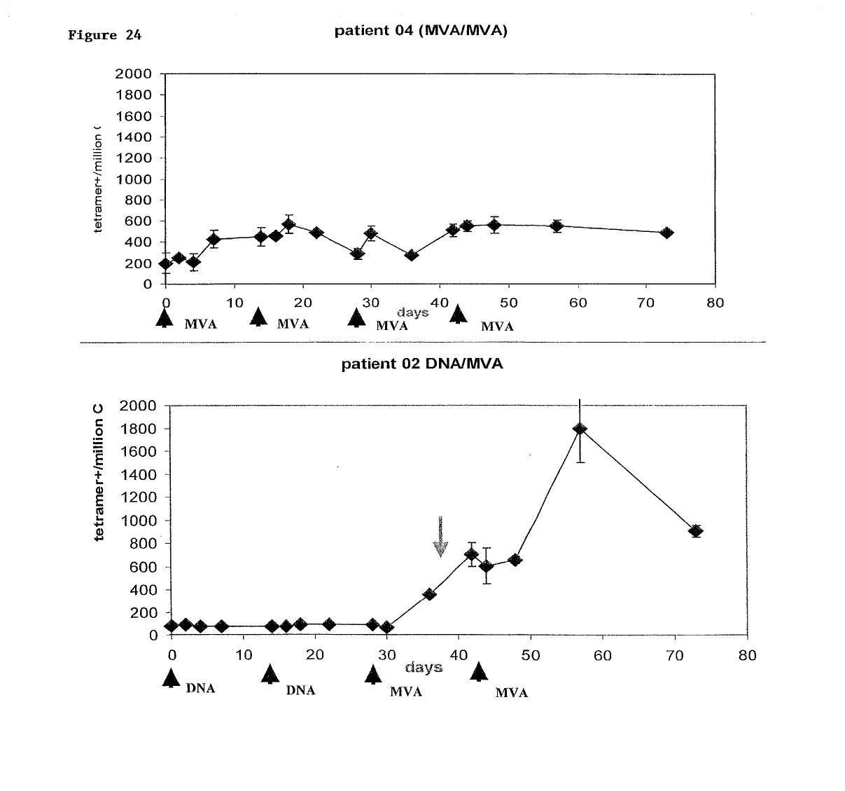

Clinical grade material of pSG2.Me13 and MVA.Me13 was produced and tested in a phase IIa trial. Stage II/III melanoma patients were treated with the following immunisations: Two injections of 1 mg of pSG2.Me13 administered intramuscularly two weeks apart followed by two intradermal injections of 5×10⁷ pfu of MVA.Me13 given two weeks apart. Melanoma-specific CD8+ T cells were detected in the peripheral blood of these melanoma patients using tetramers of HLA-A2 and melan A peptide. FIG. 24 shows the kinetics of melan-A specific CD8+ T cell expansion during the course of DNA/MVA prime-boost vaccination of a stage II melanoma patient, compared to the kinetics of melan-A specific CD8+ T cell expansion in a patient that received four intradermal injections of 5×10⁷ pfu of MVA.Me13 over the same time period. Nine days following the first MVA boosting a clinical response manifested as inflammation of naevi was noticed. These data demonstrate that a melanoma-specific CD8+ T cell response can be induced using the immunisation regime disclosed in the invention and that these CD8+ T cells do have a clinical effect.

### Example 10

### Induction of Hepatitis B Virus (HBV)-Specific CD8+ T Cells in Healthy Volunteers and Healthy Chronic HBV Carrier Using Recombinant DNA and MVA

There is evidence that a CD8+ T cell response can control or eliminate chronic viral infections. In order to treat HBV infected individuals by inducing HBV-reactive CD8+ T cells using the invention described herein, the HBV preS2-S antigen was constructed and inserted into plasmid DNA (pSG2) and MVA. The recombinant constructs were termed pSG2.HBs and MVA.HBs.

Clinical grade material of pSG2.HBs and MVA.HBs was produced and tested in a phase I trial. Healthy volunteers were treated with the following immunisations: Two injections of 1 mg of pSG2.HBs administered intramuscularly three weeks apart followed by two intradermal injections of 5×10⁷ pfu of MVA.HBs given three weeks apart.

### Example 11

### Safety and Immunogenicity of DNA/Modified Vaccinia Virus Ankara Malaria Vaccination in African Adults

Background

2-3,000 African children die each day from P. falciparum malaria and an effective vaccine is urgently needed. Several lines of evidence suggest that γ-interferon secreting T cells specific for liver-stage proteins may protect against P. falciparum. Recent advances in DNA and recombinant viral subunit vaccine studies have highlighted the DNA/MVA (modified vaccinia virus Ankara) prime-boost approach as capable of protectively immunogenic T cell induction in animal models of malaria, tuberculosis and HIV.

Methods

We investigated the safety and immunogenicity of DNA and MVA candidate vaccines against the liver-stage of P. falciparum in 20 healthy semi-immune Gambian adult males aged 18-45. The vaccines, DNA ME-TRAP and MVA ME-TRAP, both encode a whole pre-erythrocytic stage P. falciparum antigen, TRAP, and a series of T and B cell epitopes, known as the ME (multiple epitope) string. Adverse events were documented and tabulated. Immunogenicity and cross-reactivity were compared between Gambians receiving MVA ME-TRAP with (12 volunteers) and without (8 volunteers) prior DNA ME-TRAP immunisation and between Gambian and UK volunteers receiving the same regimes. Immunogenicity was assessed primarily by the γ-interferon ELISPOT assay, enumerating peptide-specific effector T cells.

Findings

As in UK volunteers there were no serious adverse events and no volunteer withdrawals due to adverse events. There were no laboratory safety abnormalities and reactogenicity was minimal. Induction of T cell responses to the ME-TRAP construct occurred in all MVA ME-TRAP immunised volunteers. DNA ME-TRAP induced low mean TRAP-specific frequencies of effector T cells in Gambian adults. MVA ME-TRAP immunisation (with or without prior DNA ME-TRAP immunisations) induced far higher mean TRAP-specific frequencies in Gambian adults. Furthermore MVA ME-TRAP is much more immunogenic in Gambian adults compared to in UK adults who received the same regime. There was much greater cross-recognition in the vaccine-induced effector T cell response for a non-vaccine strain of TRAP in malaria-exposed Gambians immunised with MVA ME-TRAP compared to malaria-naïve British volunteers. Both CD4+ and CD8+ T cells were induced by these vaccines.

Interpretation

DNA ME-TRAP and MVA ME-TRAP are safe and immunogenic in Gambian adults. T cell immunogenicity and cross-reactivity induced by MVA ME-TRAP are greater in malaria-exposed individuals than in malaria-naives, suggesting that recombinant MVA vaccines are particularly promising for malaria vaccine development for exposed populations. DNA ME-TRAP alone is weakly immunogenic and no more immunogenic in Gambian than UK adults.

Introduction

P. falciparum malaria kills one child in Africa every 30 seconds (World Health Organisation. Malaria Fact Sheet No. 94. 1996). 2 billion live in exposed regions. There are 300-500 million clinical cases and 1-2 million deaths due to malaria annually (World Health Organisation. Malaria—a global crisis. 2000; Sturchler D, Parasitology Today 1989;5:39-40). An effective malaria vaccine is urgently needed. The demonstration that DNA vaccination could induce recombinant protein expression in mouse myocytes in 1990 (Wolff J A, et al., Science 1990;247(4949 Pt 1):1465-8) heralded the prospect of DNA subunit vaccines. However DNA vaccines are suboptimally immunogenic for protection in primates (Wang R, et al, Science 1998;282(5388):476-80; Wang R, et al., Proc Natl Acad Sci USA 2001;98(19)10817-22.). If a so-called priming immunisation with a plasmid DNA encoding a pre-erythrocytic malaria antigen is followed by a boosting immunisation with a recombinant virus encoding the same antigen there is tenfold amplification of CD8+ T cell immunogenicity over DNA vaccination alone and complete protection in a mouse model of malaria (Schneider J, et al., Nat Med 1998;4(4):397-402; Plebanski M, et al, Eur J Immunol 1998;28(12):4345-55). In these experiments, protection correlated with secretion of γ-interferon by splenocytes in an ex vivo ELISPOT (enzyme-linked immunospot) assay. γ-interferon secreted by T cells induces death of intracellular liver-stage parasites by induction of reactive nitrogen intermediates within the infected cell (Ferreira A, et al., Science 1986;232(4752):881-4). The most immunogenic viral vector, in experiments by our group and others, for boosting is modified vaccinia virus Ankara (MVA), a vector suitable for human use. MVA is a highly attenuated vaccinia virus with an exceptionally good safety profile, which was developed specifically for vaccination of immunocompromised individuals during the smallpox eradication campaign (Mayr A., Berl Munch Tierarztl Wochenschr 1999; 112(9):322-8).

We have developed two novel vaccine candidates, DNA ME-TRAP and MVA ME-TRAP. The ME-TRAP construct (Gilbert S C, et al., Nat Biotechnol 1997; 15(12):1280-4) consists of the ME or multiple epitope string consisting of 18 T cell epitopes and 2 B cell epitopes and an entire well-characterised pre-erythrocytic antigen, TRAP (Robson K J, et al., Nature 1988;335(6185):79-82; Muller H M, et al., Embo J 1993;12(7):2881-9; Sultan A A, et al., Cell 1997;90(3):511-22; Flanagan K L, et al., Eur J Immunol 1999;29(6): 1943-54) (Thrombospondin-related adhesion protein). Since August 1999, in the first prime-boost trials of DNA-based vaccines in humans, we have investigated the safety, immunogenicity and efficacy of DNA ME-TRAP and MVA ME-TRAP in Phase I trials in Oxford. Over 100 volunteers have received one or both vaccines with no serious adverse events (Moorthy V S, et al., Submitted 2001).

The goal of our clinical malaria vaccine programme is the development of an effective pre-erythrocytic malaria vaccine for use in African children, who suffer greater than 90% of total worldwide mortality. In order to progress as rapidly as possible towards this goal, we are following a linked development programme in which promising regimes are evaluated in Phase I trials in African adults, staggered after Phase I evaluation in the UK. An African phase I trial in our programme to assess the potential of this new vaccine technology is described herein.

Study Setting and Volunteers

Volunteers were recruited from the peri-urban community of Bakau, on the coast of The Gambia. Approval was obtained from the Joint Gambian Government/Medical Research Council Ethics Committee and the Central Oxford Research Ethics Committee.

Potential volunteers underwent thorough clinical evaluation including a full medical history and clinical examination and were screened for haematological (fall blood count), renal (plasma creatinine, urinalysis) and hepatic (plasma alanine aminotransferase (ALT)) dysfunction. 30 adults were screened and 20 semi-immune healthy adults aged 18-45 were enrolled. All vaccinees received a standard adult dose of pyrimethamine/sulphadoxine to clear parasitaemia 14 days before first vaccination. The study was monitored throughout by an independent safety monitor based in The Gambia.

For comparison between malaria-exposed and malaria-naïve individuals, ELISPOT data from assays performed on UK volunteers are included in the analysis. The UK DNA/MVA group consists of 9 volunteers (3 volunteers in each group) who received either one, two or three 1 mg DNA ME-TRAP immunisations followed by one 5×10⁷ plaque forming units (pfu) MVA ME-TRAP immunisation (Moorthy V S, et al., Submitted 2001). The UK MVA alone group conists of 5 volunteers who received three 5×10⁷ pfu MVA immunisations. These 14 volunteers were immunised between April and August 2000. ELISPOT immunogenicity assays were performed identically by the same individuals in The Gambia and the UK.

Vaccines

The two study vaccines were DNA ME-TRAP and MVA ME-TRAP. The individual epitopes making up the ME string are described in Gilbert S C, et al., Nat Biotechnol 1997;15(12):1280-4. The strain of TRAP included in the vaccine is T9/96. The clinical vaccines were manufactured to Good Manufacturing Practice by contract manufacturers (DNA ME-TRAP by Qiagen, Germany and MVA ME-TRAP by IDT, Germany). DNA ME-TRAP was prepared in single dose vials of 1 mg in 1 ml. MVA ME-TRAP was prepared in single dose vials of 10⁸ pfu in 200 μl. The cold chain was maintained and monitored until vaccine administration.

Study Design

All vaccinations were administered at 3 week intervals. Twelve volunteers received two 1 mg doses of DNA ME-TRAP intramuscularly at weeks 0 and 3 followed by two 5×10⁷ pfu doses of MVA ME-TRAP intradermally at weeks 6 and 9. Eight volunteers received three 5×10⁷ pfu doses of MVA ME-TRAP intradermally at weeks 0, 3 and 6. Each volunteer was observed for at least one hour after vaccination. Pulse rate, blood pressure and axillary temperature were recorded immediately before and 60 minutes after vaccination. Reactogenicity assessment by a medical officer and safety and immunogenicity blood sampling occurred 7, 21 and 28 days after each vaccination and 8-10 weeks after final vaccination. A field worker visited each volunteer at home for a reactogenicity assessment on days 1-3 after each immunisation. On each of the reactogenicity assessments, whether by field worker or medical officer, the volunteer was assessed for local (pain, itching, swelling, induration, blister formation, limitation of shoulder abduction) and systemic (headache, fever, malaise, nausea or vomiting, arthralgia, myalgia) adverse events with documentation onto proforma. Each adverse event was scored using a 0-3 scoring system (0=none, 3=severe i.e. prevention of activities of daily living). All local adverse events were considered to be related to vaccination. The principal investigator (VM) assessed each systemic adverse event for relationship to vaccination as either not related, unlikely, possible or probable relationship to vaccination according to criteria in the study protocol. Any adverse event occurring within 28 days of vaccination was documented and assessed by the principal investigator for relationship to vaccination.

Laboratory Analysis

Each volunteer had 30 mls venous blood drawn from an ante-cubital vein on five occasions as follows: screening (day −28 to day −7); one week after first vaccination in MVA group only (day 7); one week after second vaccination in both groups (day 28); one week after third vaccination in both groups (day 49); one week after fourth vaccination in the DNA/MVA group only (day 70) and 8-10 weeks (day 142-156) after final vaccination. The total volume of blood taken did not exceed 150 mls over a minimum of 4 months.

Haematology. Full blood counts were measured at each timepoint using a Celloscope (Analysis Instrument AB, Bromma, Sweden). A haemoglobin concentration of >11.0 gm/dl, a white blood count between 3.5 and 11×10⁹ cells/L and a platelet count >150×10⁹/L were considered normal.

Biochemistry. Plasma creatinine (μmol/L) and ALT (IU/L) concentrations were measured at each timepoint using a chemistry analyser (Cobas Mira, ABX Diagnostics, UK). Plasma Creatinine>120 μmol/L and ALT>42 IU/L were considered abnormal.

Malaria smears. Duplicate blood films were prepared at screening and if a volunteer displayed suggestive clinical features during the trial period. These were stained with Giemsa and 200 high-power fields were read by an experienced microscopist at a final magnification of ×1,000.

Ex Vivo Enzyme-Linked Immunospot (ELISPOT) Assay: Detection of Antigen-Specific Effector T Cells

ELISPOTs were performed on Millipore MAIP S45 plates with MabTech antibodies according to the manufacturer's instructions. 4×10⁵ PBMC were incubated for 18-20 hours on the ELISPOT plates in the presence of 25 μg ml⁻¹ peptides, before being developed. The number of spot forming cells (SFCs) were counted by the AutoImmun Diagnostika system. Individual 8-mer to 17-mer epitopes were used for epitopes from the ME string, whereas 20-mers overlapping by 10 were used to span TRAP with both T9/96 and 3D7 strains of TRAP spanned in their entirety. Peptides were assayed in pools due to cell number limitations and cells were assayed in duplicate for each pool.

Characterisation Of Effectors By Specific Cell Depletions

Cell separations were performed on cells frozen in 90% Foetal Bovine Serum/10% Dimethylsulfoxide. Once thawed, washed and counted, the cells were incubated for 15 minutes at 4° C. with CD4 or CD8 Miltenyl Biotech MACS beads and then passed through a magnetic separation column. Undepleted, CD4 depleted and CD8 depleted cell populations were then assayed as above in ex vivo ELISPOT assays. Cell separations were checked by co-staining aliquots of separated (and unseparated) populations in cold PBS containing 0.5% BSA and 0.05% sodium azide with fluorescein isothiocyanate-conjugated anti-CD4 and phycoerythrin-conjugated anti-CD8 antibodies (Sowsan Atabani to add). Cells were analysed by a FACSCalibur (Becton Dickinson, San Jose, Calif.) and Cell Quest software. 10,000 live events were collected. All populations shown were separated to >90% purity.

Statistics

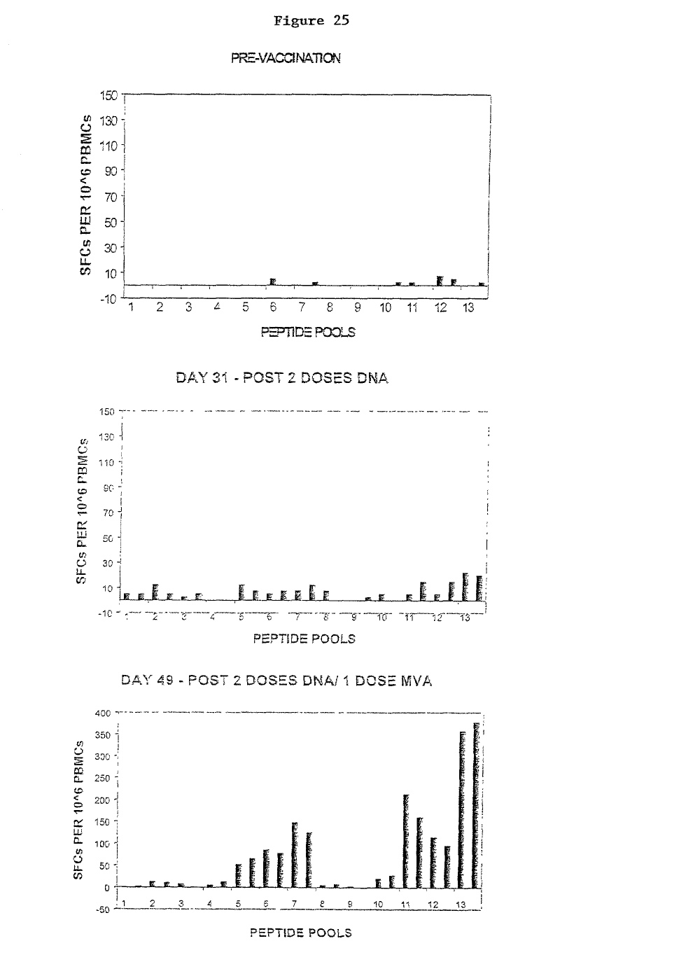

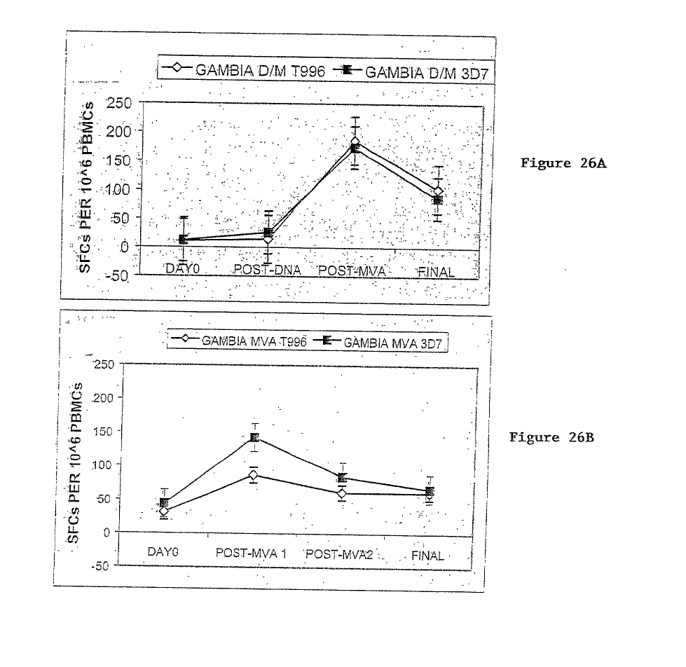

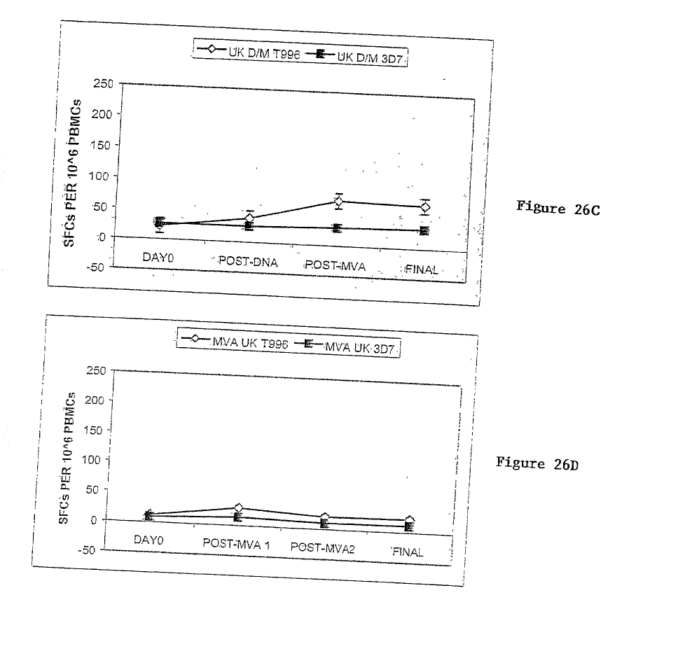

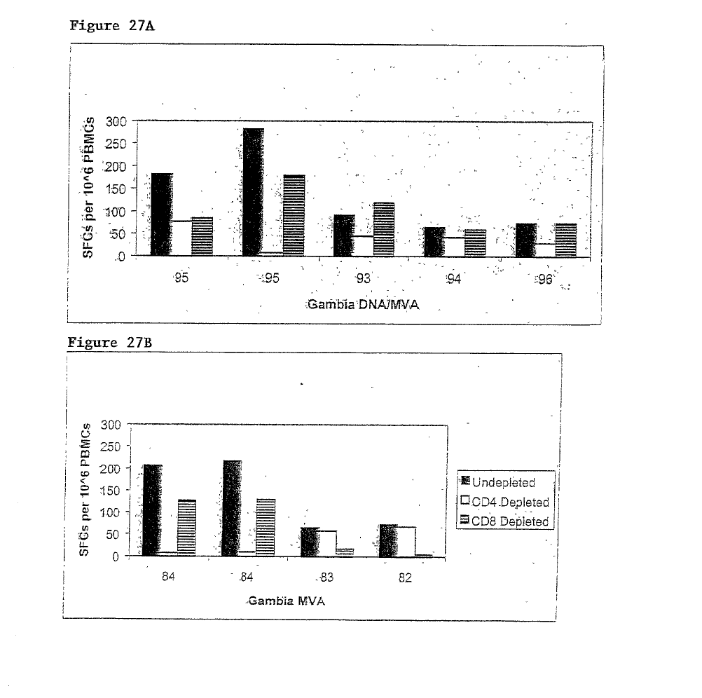

Mean T9/96- and 3D7-specific effector T cell frequencies were obtained as follows. Peptide pools 5-9 spanned 3D7 TRAP and 10-13 spanned T9/96 TRAP (FIG. 25). SFCs enumerated by automated counting were adjusted to SFCs per million PBMCs. Spots were summed across relevant pools and the “no peptide” negative control spot counts subtracted the requisite number of times. Arithmetic means were obtained of these values (FIGS. 26A-26D). For significance testing, 2-tailed student's t-tests were performed on groups of logged individual summed responses.

Results

Safety and Reactogenicity

The study was conducted between September 2000 and August 2001. Two volunteers had asymptomatic P. falciparum parasitaemia (55 and 32 parasites in 200 high power fields respectively) on screening, received treatment with chloroquine and pyrimethamine/sulphadoxine and had subsequent negative blood smears prior to vaccination. Eighteen of 20 vaccinees completed the study protocol. Two withdrew their consent after second DNA immunisation. Neither volunteer experienced any adverse events. The vaccines were well-tolerated (Table 19). All the tabulated adverse events were mild (no interference with activities of daily living). There was one moderate adverse event (some interference with but not prevention of activities of daily living) as follows: pain on abduction of the ipsilateral arm from day 3 to day 6 after first MVA immunisation after two previous DNA immunisations. Abduction was limited to 80° on examination on day 5. No axillary or supraclavicular lymph nodes were palpable. By day 7 arm movement had returned to >120°. This volunteer received simple analgesics. There were no severe or serious adverse events. There were no local adverse events after a total of 24 DNA ME-TRAP doses and no systemic adverse events assessed as being probably related to vaccination. MVA causes a characteristic local reaction after intradermal administration with discolouration and induration peaking at 48-72 hours. There is no warmth. In 5/18 MVA ME-TRAP first doses, there was a blister<2 mm at the centre of the indurated lesion, which healed without complications over 1-3 weeks in all cases. Of note, local reactions were markedly less prominent on second or third doses compared to first doses, possibly due to secondary immunity to the highly immunogenic viral vector.

Analysis of the haematology and biochemistry safety assays performed reveal no adverse events. Two volunteers experienced an episode of clinical malaria (13 and 9 asexual parasites per high power field) between screening and first DNA dose in one case and between first and second DNA doses in another case. In both instances, parasitaemia and symptoms resolved entirely after standard treatment courses of chloroquine and pyrimethamine/sulphadoxine. No other episodes of clinical malaria occurred during the study.

High-Frequency Induction of Effector T Cells Specific for T9/96 TRAP

Gambian volunteers had variable and generally very low frequencies, pre-vaccination, of circulating effector T cells specific for peptides spanning the ME-TRAP construct, although frequencies of TRAP-specific T cells were higher than in the UK. Effector T cells were induced with specificity for all peptide pools; TRAP-specific frequencies were higher than ME-specific frequencies (FIG. 25). Following DNA ME-TRAP immunisation effector cell frequency showed a small but detectable increase (p value, not significant). MVA ME-TRAP immunisation induced very much greater effector T cell frequencies than DNA ME-TRAP immunisation. Immunogenicity of MVA ME-TRAP following preceding DNA ME-TRAP (in terms of vaccine-induced T9/96 TRAP-specific effectors) was over three-fold higher in Gambian than in UK volunteers (FIG. 26A vs 26C, 175.4 vs 51.4 SFCs per million PBMCs, p value 0.04). This enhanced immunogenicity was also striking in Gambians who received MVA ME-TRAP immunisation without prior DNA ME-TRAP compared to UK adults who received the same regime (FIG. 26B vs 26D, 55.4 vs 17.2 SFCs per million PBMCs, p value 0.15). Prior DNA ME-TRAP increased T9/96 TRAP effector T cell frequency after MVA boosting in Gambians compared to Gambians who received MVA without prior DNA (FIG. 26A vs 26B, 175.4 vs 55.4 SFCs per million PBMCs, p value 0.02). Mean TRAP-specific frequencies were lower after second and third MVA immunisations than after the first and are therefore not shown. The immunogenicity of DNA ME-TRAP prior to subsequent MVA ME-TRAP boosting in Gambian adults was no higher than the weak immunogenicity of DNA ME-TRAP seen in UK adults. Effector frequencies at the 8-10 week follow-up in Gambian adults were 54-102% of the peak frequencies (94.9 and 56.7 SFCs per million PBMCs for DNA/MVA and MVA groups respectively).

Enhanced Cross-Recognition by Effector Cells for 3D7 TRAP

To assess the ability of a malaria vaccine to induce a cross-reactive T cell response we evaluated the effector response to the 3D7 strain of TRAP, a heterologous strain with 5% sequence variance at the amino acid level compared to T9/96. In the UK, there is little cross-recognition of this strain by effector cells induced by vaccination with the T9/96 TRAP encoding vaccines (FIG. 26C). Cross-recognition of responses induced in Gambians with the same vaccination regime was far in excess of that seen in UK volunteers (vaccine-induced response to 3D7 after DNA then MVA immunisation—162.3 in Gambians vs 3.2 in UK, p value 0.01, FIG. 26A vs 26C). Several volunteers demonstrated complete cross-recognition of responses in The Gambia whereas in the UK many volunteers had no detectable cross-recognition. Gambian volunteers were not stratified to vaccination group by pre-vaccination effector frequency and the group who received MVA immunisations without DNA immunisations had the higher pre-vaccination effector frequencies (p value, not significant). Prior to vaccination T cell responses in this group were higher to 3D7 than T9/96. In this group immunogenicity was correspondingly greater for 3D7 than T9/96 (FIG. 26B, vaccine-induced responses of 98.6 vs 55.4 SFCs per million PBMCs, p value not significant).

Characterisation of Vaccine-Induced Effectors

The induced effectors are of both CD4+ and CD8+ T cell subsets (FIGS. 27A-27B). Most responses were CD4+ or mixed CD4+ and CD8+ but pure CD8+ responses were also seen.

Anti-TRAP Antibodies

12 out of 20 Gambian volunteers had titres of anti-TRAP antibodies (to either or both of T9/96 and 3D7 strains) statistically significantly above titres in malaria-naives. There was no evidence of antibody induction after vaccination (unpublished data).

Discussion

Most vaccines in widespread use are formulated and delivered for optimum antibody induction. The prime-boost approach outlined in this paper is an example of a new approach targeted at maximisation of T cell immunogenicity. Potent T cell induction is likely to be necessary to effectively vaccinate against intracellular organisms such as HIV, M. tuberculosis and liver-stage P. falciparum and for cancer immunotherapy.

The ability of MVA vaccines to amplify pre-existing T cell responses induced by priming with DNA vaccines in animal models suggested that they may be more immunogenic in African volunteers who have been previously primed by natural exposure to malaria. Previously in a mouse model, it has been shown that immunogenicity of a single dose of recombinant vaccinia is not protective but prior exposure to malaria sporozoites boosts this immunogenicity to protective levels (Li S, et al., Proc Natl Acad Sci U S A 1993;90(11):5214-8), protection which is T-cell mediated. Our findings confirm that this is indeed the case. The immunogenicity of a MVA malaria vaccine (with or without DNA priming) was of greater magnitude in previously exposed Gambian individuals than in malaria-naïve British individuals. Testing efficacy of pre-erythrocytic vaccines by experimental induction of malaria in malaria-naive vaccinated volunteers may therefore underestimate protection afforded by DNA/MVA vaccines in endemic countries. This enhanced immunogenicity in malaria-exposed individuals has not been shown to occur for antibody induction with protein subunit vaccines. Immunogenicity with the candidate malaria vaccine RTS,S/AS02 was no higher in malaria-exposed adults than malaria-naives (Reece W H H, et al., Submitted 2001.). Interestingly even with sporozoite priming, further priming by DNA immunisation is still necessary for maximal T cell induction by MVA immunisation.

The greatly increased cross-recognition demonstrated in Africans in this study provides encouragement for further work with T-cell inducing DNA and recombinant viral vaccines. Lack of strain-transcendence has long been thought to be a key obstacle to malaria vaccination. The volunteers who received MVA without prior DNA vaccination in The Gambia (FIG. 26B) had higher pre-vaccination effector T cell frequencies to 3D7 TRAP than the vaccine strain T9/96. After immunisation frequencies of T cells specific for 3D7 TRAP rose more than those to T9/96, an example of original antigenic sin (Klenerman P, Zinkemagel R M. Nature 1998;394(6692):482-5).

Not only CD8+ T cells, but also high frequencies of CD4+ T cells, were induced by DNA/MVA and MVA immunisation. Whilst CD4+ T cells have traditionally been considered helper T cells for either antibody production or CD8+ T cell cytotoxicity, this distinction is no longer clear. Directly cytotoxic CD4+ T cell clones confer protection in murine adoptive transfer experiments, (Tsuji M, et al., J Exp Med 1990;172(5):1353-7.) and such CD4+ cytotoxic T cell clones are present in attenuated-sporozoite immunized protected humans (Moreno A, et al., Int-Immunol 1991;3(10):997-1003.).

In mouse models, contraction of approximately 90-95% of the effector T-cell pool by apoptosis occurs after infectious challenge over 2 weeks (for CD8+ T cells) or 7 weeks (for CD4+ T cells) (Homann D, et al., Nature Med. 2001;7:913-919.). We examined the kinetics of such contraction in humans. We demonstrated the persistence of a residual memory pool with rapid effector function 8-10 weeks after final vaccination with frequencies at this time greater than 50% of the peak frequencies.

There has been difficulty in the past in strong effector T cell induction through vaccination. We present a safe strategy which is highly immunogenic for effector T cell induction. This approach is likely to be applicable to other fields. The confirmation of the potent ability of MVA to boost pre-existing T cell responses in humans implies that MVA and the DNA/MVA combination maybe particularly effective for immunotherapy to clear or control chronic infections and halt disease progression. Examples which merit evaluation are tuberculosis infection prior to onset of disease, hepatitis B infected individuals at risk of disease progression and immunotherapy of HIV-positive and cancer patients. This does not preclude the use of DNA/MVA vaccines for prophylactic immunisation. The prospects of DNA vaccination in humans are improved by the confirmation of their ability to act as priming agents for subsequent boosting by MVA.

TABLE 19
Reactogenicity after each dose of MVA ME-TRAP

| | Dose 1 | Dose 2 | Dose 3 |
|---|---|---|---|
| Local Adverse Events | (n = 18) | (n = 18) | (n = 7) |
| Discolouration | 9 (6-18) | 7 (4-11) | 7 (5-9) |
| Itching | 7 | 3 | 1 |
| Pain | 5 | 0 | 0 |
| Blisters | 5 | 0 | 0 |

| Systemic Adverse Events | Total | PB | Total | PB | Total | PB |
|---|---|---|---|---|---|---|
| Temperature ≧ 37.5° C. | 1 | 1 | 0 | 0 | 0 | 0 |
| Headache | 2 | 1 | 1 | 1 | 2 | 1 |
| Malaise | 1 | 1 | 0 | 0 | 1 | 0 |
| Myalgia | 0 | 0 | 1 | 0 | 0 | 0 |
| Arthalgia | 1 | 1 | 0 | 0 | 0 | 0 |
| Nausea | 0 | 0 | 0 | 0 | 0 | 0 |

Data for discolouration are median (range) in mm measured on day 2. PB=probably related. Data for other fields are numbers of volunteers experiencing adverse event during 3-day follow-up. n is the number of volunteers who received each dose for whom diary cards were completed.

TABLE 20

| Volunteer | Unseparated | CD4 | CD8 |
|---|---|---|---|
| ID | PBMC | Depleted | Depleted |
| 093 DM | 92.5 | 45 | 120 |
| 094 DM | 65 | 42.5 | 60 |
| 095 DM | 182.5 | 77.5 | 85 |
| 095 DM | 282.5 | 7.5 | 180 |
| 096 DM | 75 | 30 | 75 |
| 082 M | 72.5 | 67.5 | 5 |
| 083 M | 65 | 57.5 | 17.5 |
| 084 M | 207.5 | 7.5 | 127.5 |
| 084 M | 217.5 | 10 | 130 |

### Example 12

### A DNA/MVA Prime-Boost Vaccination Induces Strong Immune Responses and Partial Protection Against Plasmodium falciparum in Humans

Animal models of vaccination against malaria indicate that the induction of CD8+ and CD4+ IFNγ T cell responses against liver stage proteins of P. falciparum can be protective. We show here that a prime-boost regimen of DNA followed by recombinant Modified Vaccinia Ankara strain (MVA) induces these T cell responses and is partially protective in humans.

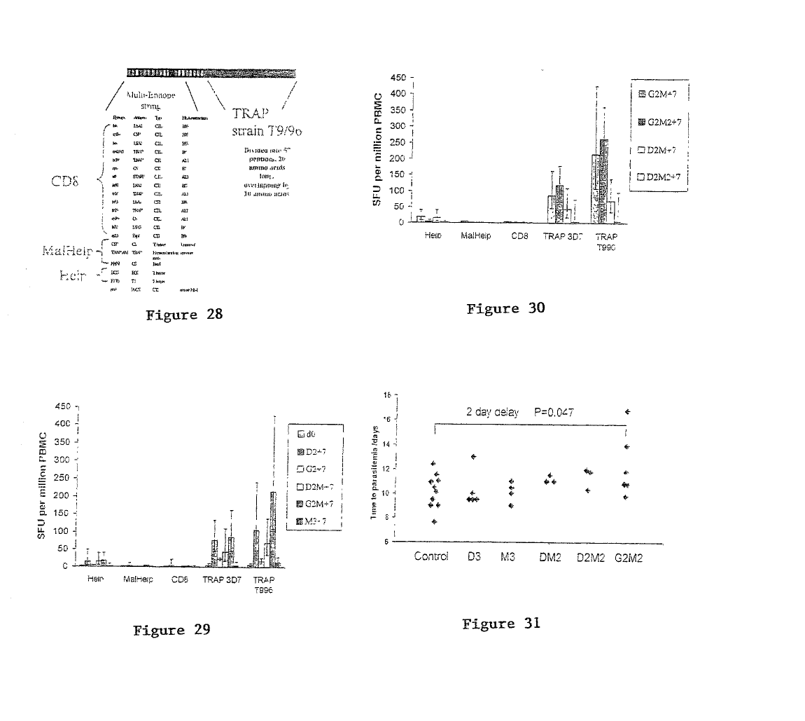

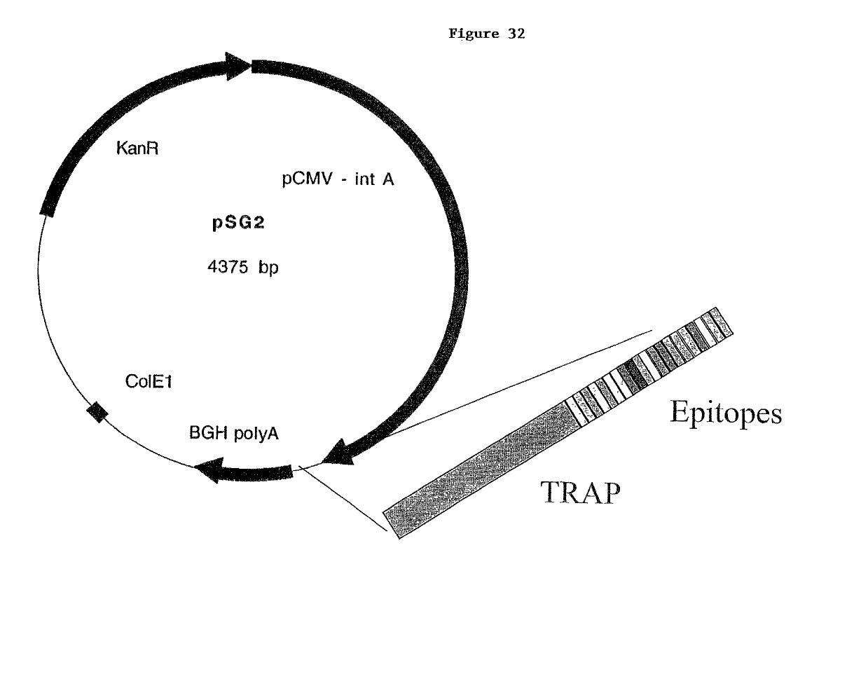

The vaccine construct (FIG. 28) was a multiepitope (ME) string fused to the Thrombospondin Related Adhesive Protein (TRAP) from P. falciparum strain T996. All epitopes were from pre-erythrocytic stages of the parasite.

Responses in an ex vivo IFNγ ELISPOT assay were weak after DNA or MVA alone, but were strongly induced when the prime-boost regime was used (FIG. 29). Successful priming was observed when 0.5 or 1 mg DNA was delivered intramuscularly, though better priming was observed with 4 μg DNA delivered by Powderject's needleless delivery device. There was no evidence of an increased response with a second booster vaccination.

Responses were induced both against TRAP from both the homologous P. falciparum strain T996 and the heterologous strain 3D7 (albeit slightly lower) (FIG. 29). The responses in 4 volunteers were CD4⁺ dependent, while in 2 they were CD8⁺ dependent.

On challenge with strain 3D7, there was a significant delay in the onset of parasitemia in two out of five donors from the group with the highest immune responses (FIG. 31). Given the high rate of division of P. falciparum at the blood stage, this indicates a large reduction in the number of parasites escaping from the liver.

These results indicate that the DNA/MVA vaccination is immunogenic in humans, and can provide protection against a large heterologous challenge.

### Example 13

### Enhanced T-Cell Immunogenicity in Humans of Plasmid DNA Vaccines Boosted by Recombinant Modified Vaccinia Virus Ankara

Immunity against pre-erythrocytic stages of Plasmodium falciparum (Pf) malaria, like that against many other uncontrolled human pathogens, may require effector T-cells. Previous attempts to induce strong cellular immune responses in humans have had limited success. We show that a heterologous prime-boost vaccination, intramuscular or intradermal DNA followed by intradermal recombinant modified vaccinia Ankara (MVA), induces powerful specific CD4+ and CD8+ T cell responses in humans to the Pf pre-erythrocytic antigen, Thrombospondin Related Anonymous Protein (TRAP). These are 7-15 fold higher than the responses after DNA alone, depending on the interval between priming and boosting. We present evidence that this can protect against heterologous sporozoite challenge. This heterologous prime-boost immunisation approach could provide a basis for successful preventative and therapeutic vaccination against many pathogens.

Malaria is an increasingly uncontrolled public health problem; 1-3 million people die annually from Pf infection according to WHO, and another 200-400 million people suffer clinical disease (Marshall, E., Science, 290:428-430 (2000)). About 20% of children with severe malaria will die of the condition, even if excellent therapeutic medical care is available (Brown, G. V., et al., Parasitol. Today, 16(10):448-451 (2000)). Thus preventive vaccination would be very attractive for its control.

Severe malaria is less likely in those with HLA B53 (Hill, A., et al., Nature, 352:595-600 (1991)), suggesting a role for Class I HLA-restricted T cells in protective immunity. Risk of both asexual Pf parasitaemia and clinical disease has been related to the number of IFN-γ producing CD4+ T-lymphocytes measured in a cultured ELISPOT assay (Reece, in press). Human irradiated sporozoite-induced immunity is associated with cellular responses (Herrington, D., et al., Am. J. Trop. Med. Hyg., 45(5):539-547 (1991)). In contrast to most traditional vaccination strategies, which are directed towards the humoral arm of the immune system, vaccine development efforts for pre-erythrocytic stages of malaria have recently been mainly directed towards inducing cellular immunity, based largely on findings in animal models (Hoffman,, S. L., et al., Science, 244(4908):1078-1081 (1989)). In inbred mice strains, various types of specific immune cells are important, most frequently CD8+ T-cells, but also CD4+ T-cells, creating diverse semi-redundant parallelism (Doolan, D. L., et al., J. Immunol., 165(3):1453-1462 (2000)).

The strength of the cellular immune response in humans after DNA vaccines alone has been disappointing (Wang, R., et al., Science, 282(5388):476-480 (1998); Wang, R., et al., PNAS, 98:10817-10822 (2001)). Animal models of several pathogens suggest that combinations of different vaccines in a heterologous prime-boost strategy are much more potent (Schneider, J., et al., Nature Med., 4:397-402 (1998); Hanke, T., et al., Vaccine, 16(5):439-445 (1998): McShane, H., et al., Infect. Imm., 69(2):681686 (2001); Schneider, J., et al., Vaccine, 19(32):4595-4602 (2001); Sullivan, N. J., et al., Nature, 408(6812):605-609 (2000)) especially using a poxvirus boost.

We have designed vaccines against the pre-erythrocytic stages of P. falciparum, using as vectors plasmid DNA and recombinant modified vaccinia virus Ankara (MVA). Here we describe their evaluation in a series of sequential small clinical trials showing that they are well tolerated, highly T cell immunogenic and partly effective in controlling malaria in a high dose human challenge model using a heterologous parasite strain.

The malarial DNA sequence was full-length Pf TRAP of strain T9/96 (Robson, K. J., et al., Nature, 335(6185):79-82 (1988) fused to a string of 20 selected T-cell and B-cell epitopes (ME) (Table 21) (Gilbert, S. C., Nat. Biotechnol., 15(12):1280-1284 (1997)). The epitopes but not the TRAP antigen were recoded towards mammalian codon bias.

Healthy adult volunteers resident in Oxford were recruited (Moorthy, V. S. in press (2001)) and immunised with plasmid DNA (Within the kanamycin resistant plasmid ME-TRAP hybrid was regulated by a CMV IE promoter with intron A for expression in eukaryotic cells and bovine growth hormone derived polyadenylation transcription terminator. DNA ME-TRAP was produced under good manufacturing practices by Qiagen GmbH (Hilden, Germany) (Schneider, J., et al., Nature Med., 4:397-402 (1998)) and MVA (The ME-TRAP hybrid DNA was ligated into the vaccinia shuttle vector pSC11 bringing it under control of the vaccinia P7.5 early promoter. This vector includes the E. coli beta-galactosidase gene expressed by the vaccinia P11 late promoter. The region including ME-TRAP and the beta-galactosidase gene is flanked by sequences from the vaccinia thymidine kinase locus to allow insertion into the vaccinia genome. Chicken embryo fibroblast (CEF) cells infected with wild type MVA virus were transfected with pSC11 ME-TRAP. Recombinant virus was isolated using B-galactosidase substrate X-gal overlay of infected CEF monolayers (Chakrabarti, S., et al., Mol. Cell Biol., 5(12):3403-3409 (1985)) vaccines, recombinant for the ME-TRAP fusion protein, individually and in prime-boost combinations at a range of doses. The vaccination schedule of each arm of each trial is described in Table 22. Briefly, 3 groups received DNA only, 4 groups MVA only, 8 groups DNA prime and MVA boost. Of these, 7 groups had sporozoite challenge as described below. DNA ME-TRAP was given either intramuscularly at doses of 500, 1000 or 2000 micrograms or intradermally by a needleless delivery device at a dose of 4 micrograms (Powderject, Oxford) (Roy, M. J., et al., Vaccine, 19(7-8):764-778 (2000)). MVA.ME-TRAP was given by intradermal injections of 100 microlitre aliquots into the skin over one or both deltoid areas at doses of 3, 6 or 15×10⁷ plaque forming units (pfu).

No serious AEs occurred after any vaccinations (Moorthy, V. S., et al. in press (2001). Intramuscular DNA vaccination was not associated with any localised AEs. No anti-nuclear antibodies were detected after vaccination.

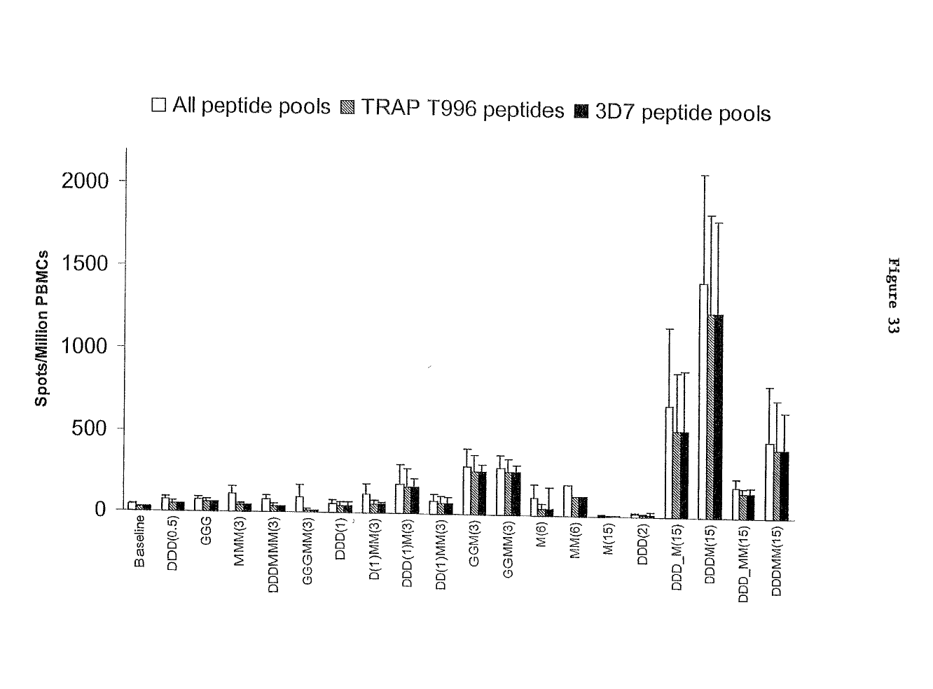

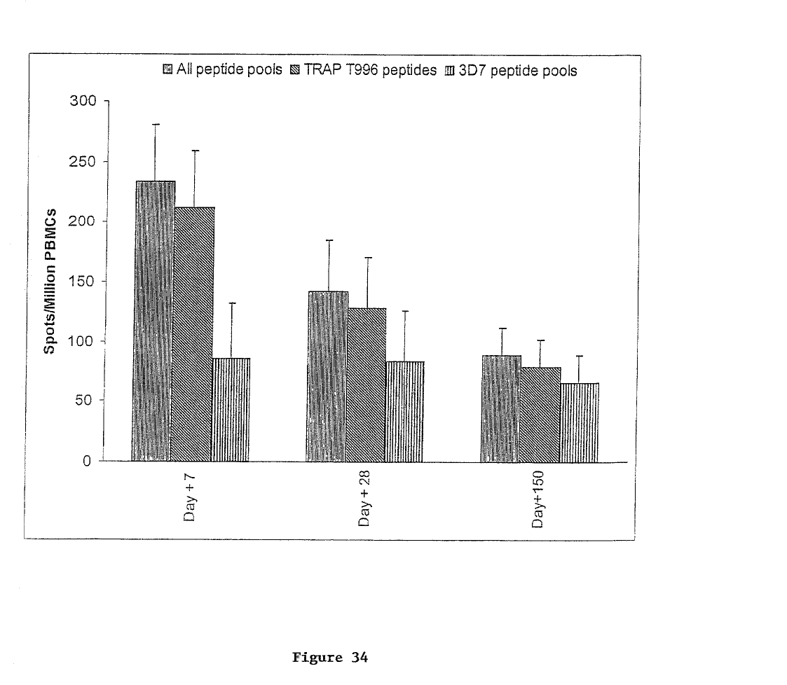

Vaccination with DNA alone produced small responses in the ex vivo ELISPOT assay (p=0.03) but its use before MVA.ME-TRAP caused a massive increase in the responses (Table 23 and FIG. 33) (The main immunological measure is the ex vivo interferon-γ ELISPOT response, that correlates with protection in mouse sporozoite challenge studies, and this was performed at abseline, 7, 21-28 and 130-150 days after vaccination. These were performed on fresh peripheral-blood mononuclear cells (PMBC) using pools of 20-mer peptides that span the length of TRAP and overlap by 10 amino acids (Reece, in press). The known epitopes in the ME string were also tested in pools. Briefly, 400,000 PBMC per well were plated directly onto the ELISPOT plaste in the presence of 25 μg ml-1 or peptide, and incubated for 18 h. ELISPOT responses to TRAP peptides of the vaccine strain, T996, and to the challenge strain, 3D7, were assessed separately. Antibodies to the CSP NANP repeat sequence and to TRAP of both the 3D & and T9/96 strains were measured by ELISA. The ELISPOT data was analysed by subtracting the number of spots in the wells with medium and cells alone from the coutns of spots in wells with pools of peptides and cells. Counts less than zero wree disregarded. The results were summed across all the peptide pools. Geometric means of the summed peptide-specific spots are presented. ANOVA for repeated measurements was used to compare between groups). After vaccination the summed net spots in ELISPOT wells to peptides from Pf T9/96-strain TRAP in subjects who had a DNA-prime followed by MVA showed a significant change from baseline (p=0.0006, with adjustment for multiple comparisons). The heterologous responses to pools of peptides from Pf3D7-strain TRAP were lower but still changed from baseline, and CD8+ T-cell-restricted responses to the ME string were significant in the groups who received 2 mgs DNA per dose and a subsequent MVA boost at 15×10e7 pfu. The doses of both DNA and MVA are crucial; higher doses were associated with much higher responses. Intradernal delivery of 4 μg DNA by needleless device is more immunogenic than intramuscular delivery of 1 mg DNA. The immunological responses after the shorter interval of 3 weeks between DNA and MVA were stronger (p=0.026) than after an 8 week interval. The T-cell responses waned over time but were still 38% of the peak after 5-11 months and 61% of the initial memory-pool level (day 21-28) at the 5-11 month time point (FIG. 34).

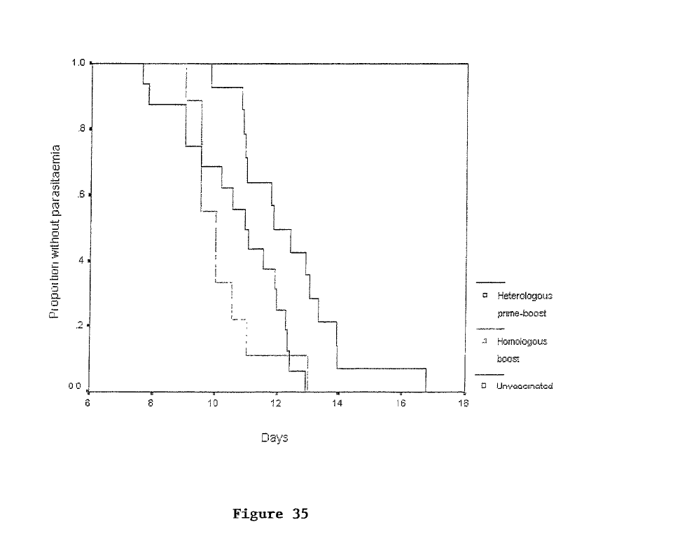

The Pf sporozoite challenge model we adopted was described by Chulay et. al. (Chulay, J. D., et al., Am. J. Trop. Med. Hyg., 35(1):66-68 (1986)) based on advances in gametocyte culture and membrane feeding (Ponnudurai, T., et al., Trans. R. Soc. Trop. Med. Hyg., 76(2):242-250 (1982)). (Five An. Stephensi mosquitos each with 10²-10⁴ sporozoites per salivary gland were allowed to bite each subject thus delivering 3D7 Pf sporozoites. Monitoring took place twice daily using Giesma-stained thick blood films starting on day 5. Subjects were treated with chloroquine after the first confirmed positive blood film. The five or six unvaccinated control subjects in each challenge trial all developed parasitaemia 8-13 days later. The time to parasitaemia in control subjects and vaccinated subjects was compared using the log rank test.) The sporozoite challenge results indicate that volunteers in the GGMM(3), DDD_MM(15) (8 week interval between third D and first M) and DDDMM(15) (3 week interval) groups have a significant delay in time to parasitaemia (p=0.013) as illustrated in FIG. 35. This indicates that the vaccines induce an effective immune response against pre-erythrocytic Pf parasites.

In the GGMM(3) group depletion assays were performed on cryopreserved cells which showed that 4 of the subjects had CD4+ T-lymphocyte dependent responses and 2 had CD8+ T-lymphocyte dependent responses. In the DDDMM(15) and DDD_MM(15) groups that had the largest responses, depletion studies, done on fresh cells indicated that all the responses were CD4+ T-lymphocyte dependent, including the low level responses seen to the nonamer and decamer peptides within the ME string. Therefore we have induced CD4+ T-cell dependent CD8+ T-lymphocyte responses.

We attempted to find specific immunological responses that correlate with the protection but this approach is limited by the small sample size of those partly protected. None the less, responses of some peptide pools, for example, pools 31-40 from TRAP, corresponding to amino acids 300-410, showed a significant correlation with time to parasitaemia. There were no detectable antibodies present to TRAP protein. Three subjects developed antibodies to the NANP repeat epitope in the vaccine.

This is the first demonstration of powerful effective effector T-cell responses in humans after vaccination. The frequency of circulating effector T cells, as measured by ex vivo ELISPOT was much higher than in other vaccination studies in humans. For example, after RTS,S/AS02 malaria vaccinination the comparable geometric mean response in the most responsive subgroup was about 20 cells/million peripheral blood mononuclear cells (PBMC) (Lalvani, A., et al., JID, 180:1656-1664 (1999)). A DNA vaccine for HBV elicited protective levels of antibodies and some cellular responses (Roy, M. J., et al. Vaccine, 19(7-8):764-778 (2000)). Another malaria DNA vaccine shows similar immunogenicity in ex vivo ELISPOT to DNA ME-TRAP; 7 or 15 fold lower than DNA/MVA prime-boost immunisation with DDD_M(15) or DDDM(15) respectively (Wang, R., et al., PNAS, 98:10817-10822 (2001)). Earlier attempts to elicit cellular immunity required pre-stimulation of lymphocytes to elicit detectable responses (Wang, R., et al., Science, 282(5388):476-480(1998)).

The results indicate that, as in animals, DNA priming followed by MVA-boosting vaccination produces large cellular responses, which far surpass the responses seen after either vaccine alone. The responses are of the same order as those seen in mice or chimpanzees (Schneider, J., et al., Nature Med., 4:397-402 (1998; Schneider, J., et al., Vaccine, 19(32):4595-4602 (2001)).

This was the first time that heterologous challenge was used in a subunit malaria vaccine trial. The TRAP amino acid sequences from T9/96 and 3D7 strains of P. falciparim show 6.1% sequence variation. The bites of five mosquitoes with heavily infected salivary glands in this model probably delivers very substantially more sporozoites than bites during natural exposure in the field. The results in the rigorous heterologous sporozoite challenge presented here are very encouraging that the immune responses that we are detecting are associated with efficacy. Protection is statistically significant and indicates that an appropriate effector arm of the immune system has been stimulated by the vaccine. Protection may be superior in a field setting with lower levels of sporozoite inoculation per infection.

These are the first polyepitope vaccines to be evaluated in humans. The TRAP protein contains many more potential epitopes. A large number of dominant epitopes in TRAP may have decreased the responses to the epitope string, which were low. The strong ELISPOT responses we found in unstimulated PBMCs suggest that the TRAP protein coded by the vaccines was the protective antigen.

We have shown that vaccination using the TRAP sequence from T9/96 strain of pf generates peptide specific T-cells that respond to TRAP peptides from the heterologous 3D7 pf strain used in the challenge. This indicates a possible biological basis of cross-protection against other strains of P. falciparum. The persistence of the responses were also impressive, as the levels at day +150 were 61% of the day 21-28 levels.

Animal data suggests that recombinant MVA is a particularly effective agent for boosting T-cells responses (Hanke, T., et al., Vaccine, 16(5):439-445 ((1998); Schneider, J., et al., Vaccine, 19(32):4595-4602 (2001)). We show that a single dose of recombinant MVA is adequate and suggest that little benefit is gained from a further booster immunisation. It is an increasingly promising viral vector due to its marked immunogenicity when used as a boosting agent, the excellent safety profile in an immunocompromised monkey model (Vaccine August 2001) and its safety in humans (Moorthy, in press, 2001).

The vaccination strategy described above could be the basis for effective vaccines for malaria, HIV, Hepatitis B virus, tuberculosis and tumours.

TABLE 21
Composition of the antigen in the vaccines

| | | | HLA |
|---|---|---|---|
| Epitope | Antigen | Type | Restriction |
| Ls8 | LSA1 | CD8 | B35 |
| cp26 | CSP | CD8 | B35 |
| ls6 | LSA1 | CD8 | B53 |
| tr42/43 | TRAP | CD8 | B8 |
| tr39 | TRAP | CD8 | A2.1 |
| cp6 | CSP | CD8 | B7 |
| st8 | STARP | CD8 | A2.2 |
| ls50 | LSA1 | CD8 | B17 |
| tr26 | TRAP | CD8 | A2.1 |
| ls53 | LSA1 | CD8 | B58 |
| tr29 | TRAP | CD8 | A2.2 |
| cp39 | CSP | CD8 | A2.1 |
| la72 | LSA3 | CD8 | B8 |
| ex23 | Exp1 | CD8 | B58 |
| CSP | CSP | T helper | Multiple |
| TRAP AM | TRAP | Heparin | Multiple |
| | | binding motif | |
| (NANP)n | CSP | B cell | Multiple |
| BCG | BCG | T helper | |
| FTTp | TT | T helper | |
| Pb69 | PbCS | CTL | murine H2-K |

| | |
|---|---|
| TRAP | Whole protein from T9/96 strain |

TABLE 22
Vaccination Schedule of Subjects

| | Vaccine | DNA | | | Interval | MVA | | | Interval | Challenge |
|---|---|---|---|---|---|---|---|---|---|---|
| Group | Group Size | Dose μg | Dose μg | Dose μg | to Boost | × 107 pfu | × 107 pfu | × 107 pfu | to Challenge | Group Size |
| DDD(0.5) | 3 | 500 | 500 | 500 | | | | | | |
| GGG | 4 | 4 | 4 | 4 | | | | | | |
| M | 1 | | | | | 3 | | | | |
| MMM(3) | 10 | | | | | 3 | 3 | 3 | 3 | 4 |
| DDDMM(3) | 3 | 500 | 500 | 5000 | 6-12 | 3 | 3 | 3 | | |
| GGGMM(3) | 2 | 4 | 4 | 4 | 14 | 3 | 3 | | | |
| DDD(1) | 5 | 1000 | 1000 | 1000 | | | | | 3 | 5 |
| D(1)MM(3) | 3 | 1000 | | | 3 | 3 | 3 | | 3 | 3 |
| DDD(1)M(3) | 3 | 1000 | 1000 | 1000 | 3 | 3 | | | | |
| DD(1)MM(3) | 3 | 1000 | 1000 | | 3 | 3 | 3 | | 5.4 | 3 |
| GGMM(3) | 6 | 4 | 4 | | 3 | 3 | 3 | | 5.4 | 6 |
| MM(6) | 3 | | | | | 6 | 6 | | | |
| MM(15) | 3 | | | | | 15 | 15 | | | |
| DDD_MM(15) | 5 | 2000 | 2000 | 2000 | 8 | 15 | 15 | | 3 | 5 |
| DDDMM(15) | 4 | 2000 | 2000 | 2000 | 3 | 15 | 15 | | 3 | 4 |

Repeat doses of the same vaccine were given after 3 week interval
Intervals are measured in weeks
D = DNA.ME-TRAP give by intramuscular injection into deltoid muscle
G = DNA.ME-TRAP given intradermally by needleless delivery device
M = MVA.ME-TRAP given by intradermal injection
pfu = plaque forming unit

TABLE 23
ELISPOT responses in peripheral blood seven days after various vaccination regimens

| | | All peptides in vaccines | | | | T9/96 TRAP | | | | 3D7 TRAP | | | |
|---|---|---|---|---|---|---|---|---|---|---|---|---|---|
| Vaccine Regimen | Group Size | Mean | SE | Geomean | SE | Mean | SE | Geomean | SE | Mean | SE | Geomean | SE |
| Baseline | 65 | 43 | 7 | 18 | 4 | 25 | 5 | 9 | 2 | 33 | 6 | 15 | 3 |
| DDD(0/5) | 4 | 73 | 18 | 66 | 19 | 48 | 20 | 33 | 23 | | | | |
| G | 10 | 112 | 36 | 65 | 29 | 78 | 30 | 35 | 19 | 13 | 5 | 9 | 7 |
| GG | 10 | 91 | 30 | 50 | 23 | 57 | 21 | 31 | 14 | 17 | 5 | 14 | 6 |
| GGG | 4 | 72 | 20 | 63 | 24 | 58 | 19 | 45 | 26 | | | | |
| MMM(3) | 9 | 110 | 48 | 44 | 36 | 41 | 13 | 24 | 14 | 16 | 3 | 14 | 4 |
| DDDMMM(3) | 3 | 77 | 24 | 70 | 25 | 38 | 18 | 28 | 23 | | | | |
| GGGMM(3) | 2 | 92 | 78 | 50 | 12 | 15 | 8 | 13 | 9 | | | | |
| | | | | | 0 | | | | | | | | |
| D(1) | 13 | 34 | 11 | 18 | 8 | 19 | 6 | 11 | 4 | 22 | 6 | 13 | 5 |
| DD(1) | 9 | 74 | 35 | 27 | 21 | 60 | 28 | 21 | 16 | 38 | 17 | 14 | 10 |
| DDD(1) | 8 | 55 | 23 | 33 | 16 | 44 | 21 | 25 | 12 | 55 | 23 | 28 | 17 |
| D(1)MM(3) | 3 | 112 | 68 | 69 | 78 | 55 | 24 | 43 | 29 | 25 | 13 | 16 | 18 |
| DDD(1)M(3) | 3 | 180 | 122 | 104 | 11 | 162 | 11 | 90 | 10 | 75 | 52 | 46 | 45 |
| | | | | | 8 | | 2 | | 9 | | | | |
| DD(1)MM(3) | 3 | 79 | 41 | 51 | 56 | 69 | 41 | 21 | 76 | 56 | 36 | 18 | 59 |
| GGM(3) | 6 | 297 | 108 | 170 | 12 | 266 | 10 | 148 | 11 | 127 | 41 | 87 | 46 |
| | | | | | 4 | | 0 | | 0 | | | | |
| GGMM(3) | 6 | 288 | 83 | 234 | 77 | 265 | 80 | 212 | 73 | 128 | 44 | 85 | 47 |
| M(6) | 2 | 109 | 85 | 68 | 12 | 44 | 27 | 35 | 36 | 208 | 12 | 162 | 173 |
| | | | | | 6 | | | | | | 9 | | |
| MM(6) | 1 | 195 | | 195 | | 119 | | 117 | | 139 | | 138 | |
| M(15) | 2 | 5 | 4.9 | 3 | 7 | 3 | 3 | 2 | 4 | 12 | 5 | 11 | 6 |
| MM(15) | 0 | | | | | | | | | | | | |
| D(2) | 7 | 22 | 2 | 21 | 2 | 13 | 3 | 10 | 5 | 25 | 5 | 21 | 7 |
| DDD(2) | 8 | 19 | 5.7 | 14 | 5 | 12 | 4 | 8 | 3 | 33 | 19 | 14 | 9 |
| DDD_M(15) | 4 | 684 | 474 | 372 | 28 | 528 | 35 | 302 | 22 | 461 | 36 | 195 | 194 |
| | | | | | 9 | | 0 | | 6 | | 3 | | |
| DDDM(15) | 4 | 1430 | 654 | 708 | 10 | 1249 | 59 | 617 | 88 | 1078 | 55 | 363 | 881 |
| | | | | | 30 | | 3 | | 0 | | 5 | | |
| DDD_MM(15) | 5 | 188 | 53 | 158 | 53 | 150 | 29 | 137 | 34 | 118 | 35 | 98 | 36 |
| DDDMM(15) | 2 | 470 | 340 | 316 | 47 | 422 | 30 | 295 | 43 | 294 | 23 | 182 | 340 |
| | | | | | 1 | | 4 | | 5 | | 1 | | |

Some subjects are included more than once as the results indicate their time course through trials. The number of subjects in each arm and their vaccination schedule is shown in Table 22. Arithmetic and geometric (geomean) means and standard error (SE) are shown for three sets of peptide pools: the summed net responses to all the epitopes in the vaccines, the summed net responses to all peptide pools from T9/96 strain of TRAP and the summed net responses to all peptide pools from 3D7 strain of TRAP.

For some time points the data is missing due to subjects' unavailability, errors in performing the assay or background responses more than 50 spots/million PBMC.

While this invention has been particularly shown and described with references to preferred embodiments thereof it will be understood by those skilled in the art that various changes in form and details may be made therein without departing from the scope of the invention encompassed by the appended claims.

What is claimed is:

1. A method of inducing a CD4+ T-cell response against a target antigen in a mammal, which comprises the step of administering at least one dose of:
(a) a first composition comprising a source of one or more CD4+ T cell epitopes of the target antigen;
and at least one dose of
(b) a second composition comprising a source of one or more CD4+ T cell epitopes of the target antigen, including at least one CD4+ T cell epitope which is the same as a CD4+ T cell epitope of the first composition,
wherein the source of CD4+ epitopes for the first and/or second composition is a non-replicating or replication impaired recombinant poxvirus vector, and wherein the doses of the first and second compositions may be administered in either order.

2. A method according to claim 1, wherein the target antigen is derivable from an infectious pathogen.

3. A method according to claim 1, wherein the target antigen is a tumour antigen or autoantigen.

4. A method according to claim 1, wherein the recombinant poxvirus vector is a modified vaccinia virus Ankara strain or derivative thereof.

5. A method according to claim 1, wherein the recombinant poxvirus vector is a fowlpox vector or derivative thereof.

6. A method according to claim 1, where the epitopes in or encoded by the first or second composition are provided in a sequence which does not occur naturally as the expressed product of a gene in the parental organism from which the target antigen may be derived.

7. A method according to claim 1, wherein the induced CD4 T cell response is of a T_{H}1-type.

8. A method according to claim 1, wherein the induced CD4 T cell response is of a gamma-interferon-secreting type.

9. A method according to any claim 1, in which at least one dose of a composition is administered by an intradermal route.

10. A method according to claim 1, which comprises administering at least one dose of the first composition, followed by at least one dose of the second composition.

11. A method according to claim 10, which comprises administering a plurality of sequential doses of the first composition, followed by at least one dose of the second composition.

12. A method according to claim 11, which comprises administering three sequential doses of the first composition, followed by one dose of the second composition.

13. A method according to claim 1, with the proviso that if the source of epitopes in (a) is a viral vector, the viral vector in (b) is derived from a different virus.

14. A method according to claim 1 wherein the target antigen is derived from a disease selected from the group consisting of: tuberculosis, HIV, malaria. H. pylori, influenza, hepatitis, CMV, viral infection, herpes virus-induced disease, leprosy, a disease caused by a non-malarial protozoan parasite such as toxoplasma, and cancer.

15. A method inducing a combined CD4+ and CD8+ T-cell response against a target antigen in a mammal, which comprises the step of administering at least one dose of:
(a) a first composition comprising a source of one or more CD4+ T cell epitopes and a source of one or more CD8+ T cell epitopes of the target antigen;
and at least one dose of
(b) a second composition comprising
(i) a source of one or more CD4+ T cell epitopes of the target antigen, including at least one CD4+ T cell epitope which is the same as a CD4+ T cell epitope of the first composition; and
(ii) a source of one or more CD8+ T cell epitopes of the target antigen, including at least one CD8+ T cell epitope which is the same as a CD8+ T cell epitope of the first composition
wherein the source of CD4+ and CD8+ epitopes for the first and/or second composition is a non-replicating or replication impaired recombinant poxvirus vector; and wherein the doses of the first and second compositions may be administered in either order.

16. A method according to claim 15, wherein the target antigen is derivable from an infectious pathogen.

17. A method according to claim 15, wherein the target antigen is a tumour antigen or autoantigen.

18. A method according to claim 15, wherein the recombinant poxvirus vector is a modified vaccinia virus Ankara strain or derivative thereof.

19. A method according to claim 15, wherein the recombinant poxvirus vector is a fowlpox vector or derivative thereof.

20. A method according to claim 15, where the epitopes in or encoded by the first or second composition are provided in a sequence which does not occur naturally as the expressed product of a gene in the parental organism from which the target antigen may be derived.

21. A method according to claim 15, wherein the induced CD4 T cell response is of a T_{H}1-type.

22. A method according to claim 15, wherein the induced CD4 T cell response is of a gamma-interferon-secreting type.

23. A method according to any claim 15, in which at least one dose of a composition is administered by an intradermal route.

24. A method according to claim 15, which comprises administering at least one dose of the first composition, followed by at least one dose of the second composition.

25. A method according to claim 24, which comprises administering a plurality of sequential doses of the first composition, followed by at least one dose of the second composition.

26. A method according to claim 25, which comprises administering three sequential doses of the first composition, followed by one dose of the second composition.

27. A method according to claim 15, with the proviso that if the source of epitopes in (a) is a viral vector, the viral vector in (b) is derived from a different virus.

28. A method according to claim 15 wherein the target antigen is derived from a disease selected from the group consisting of: tuberculosis, HIV, malaria. H. pylori, influenza, hepatitis, CMV, viral infection, herpes virus-induced disease, leprosy, a disease caused by a non-malarial protozoan parasite such as toxoplasma, and cancer.

29. A method of inducing a CD4+ T-cell response against tuberculosis in a mammal, which comprises the step of administering at least one dose of:
(a) a first composition comprising a source of one or more CD4+ T cell epitopes of tuberculosis;
and at least one dose of
(b) a second composition comprising a source of one or more CD4+ T cell epitopes of tuberculosis, including at least one CD4+ T cell epitope which is the same as a CD4+ T cell epitope of the first composition,
wherein the source of CD4+ epitopes for the first and/or second composition is a non-replicating or replication impaired recombinant poxvirus vector, and wherein the doses of the first and second compositions may be administered in either order.

30. A method according to claim 29, wherein the recombinant poxvirus vector is a modified vaccinia virus Ankara strain or derivative thereof.

31. A method according to claim 29, wherein the recombinant poxvirus vector is a fowlpox vector or derivative thereof.

32. A method according to any claim 29, in which at least one dose of a composition is administered by an intradermal route.

33. A method according to claim 29, which comprises administering at least one dose of the first composition, followed by at least one dose of the second composition.

34. A method according to claim 33, which comprises administering a plurality of sequential doses of the first composition, followed by at least one dose of the second composition.

35. A method according to claim 34, which comprises administering three sequential doses of the first composition, followed by one dose of the second composition.

36. A method according to claim 29, with the proviso that if the source of epitopes in (a) is a viral vector, the viral vector in (b) is derived from a different virus.

37. A method inducing a combined CD4+ and CD8+ T-cell response against tuberculosis in a mammal, which comprises the step of administering at least one dose of:
(a) a first composition comprising a source of one or more CD4+ T cell epitopes and a source of one or more CD8+ T cell epitopes of tuberculosis;
and at least one dose of
(b) a second composition comprising
(i) a source of one or more CD4+ T cell epitopes of tuberculosis, including at least one CD4+ T cell epitope which is the same as a CD4+ T cell epitope of the first composition; and
(ii) a source of one or more CD8+ T cell epitopes of the target antigen, including at least one CD8+ T cell epitope which is the same as a CD8+ T cell epitope of the first composition
wherein the source of CD4+ and CD8+ epitopes for the first and/or second composition is a non-replicating or replication impaired recombinant poxvirus vector; and wherein the doses of the first and second compositions may be administered in either order.

38. A method according to claim 37, wherein the recombinant poxvirus vector is a modified vaccinia virus Ankara strain or derivative thereof.

39. A method according to claim 37, wherein the recombinant poxvirus vector is a fowlpox vector or derivative thereof.

40. A method according to any claim 37, in which at least one dose of a composition is administered by an intradermal route.

41. A method according to claim 37, which comprises administering at least one dose of the first composition, followed by at least one dose of the second composition.

42. A method according to claim 41, which comprises administering a plurality of sequential doses of the first composition, followed by at least one dose of the second composition.

43. A method according to claim 42, which comprises administering three sequential doses of the first composition, followed by one dose of the second composition.

44. A method according to claim 37, with the proviso that if the source of epitopes in (a) is a viral vector, the viral vector in (b) is derived from a different virus.

45. A method of inducing a CD4+ T-cell response against malaria in a mammal, which comprises the step of administering at least one dose of:
(a) a first composition comprising a source of one or more CD4+ T cell epitopes of malaria;
and at least one dose of
(b) a second composition comprising a source of one or more CD4+ T cell epitopes of malaria, including at least one CD4+ T cell epitope which is the same as a CD4+ T cell epitope of the first composition,
wherein the source of CD4+ epitopes for the first and/or second composition is a non-replicating or replication impaired recombinant poxvirus vector, and wherein the doses of the first and second compositions may be administered in either order.

46. A method according to claim 45, wherein the recombinant poxvirus vector is a modified vaccinia virus Ankara strain or derivative thereof.

47. A method according to claim 45, wherein the recombinant poxvirus vector is a fowlpox vector or derivative thereof.

48. A method according to any claim 45, in which at least one dose of a composition is administered by an intradermal route.

49. A method according to claim 45, which comprises administering at least one dose of the first composition, followed by at least one dose of the second composition.

50. A method according to claim 45, which comprises administering a plurality of sequential doses of the first composition, followed by at least one dose of the second composition.

51. A method according to claim 45, which comprises administering three sequential doses of the first composition, followed by one dose of the second composition.

52. A method according to claim 51, with the proviso that if the source of epitopes in (a) is a viral vector, the viral vector in (b) is derived from a different virus.

53. A method inducing a combined CD4+ and CD8+ T-cell response against malaria in a mammal, which comprises the step of administering at least one dose of:
(a) a first composition comprising a source of one or more CD4+ T cell epitopes and a source of one or more CD8+ T cell epitopes of malaria;
and at least one dose of
(b) a second composition comprising
(i) a source of one or more CD4+ T cell epitopes of malaria, including at least one CD4+ T cell epitope which is the same as a CD4+ T cell epitope of the first composition; and
(ii) a source of one or more CD8+ T cell epitopes of malaria, including at least one CD8+ T cell epitope which is the same as a CD8+ T cell epitope of the first composition
wherein the source of CD4+ and CD8+ epitopes for the first and/or second composition is a non-replicating or replication impaired recombinant poxvirus vector; and wherein the doses of the first and second compositions may be administered in either order.

54. A method according to claim 53, wherein the recombinant poxvirus vector is a modified vaccinia virus Ankara strain or derivative thereof.

55. A method according to claim 53, wherein the recombinant poxvirus vector is a fowlpox vector or derivative thereof.

56. A method according to any claim 53, in which at least one dose of a composition is administered by an intradermal route.

57. A method according to claim 53, which comprises administering at least one dose of the first composition, followed by at least one dose of the second composition.

58. A method according to claim 57, which comprises administering a plurality of sequential doses of the first composition, followed by at least one dose of the second composition.

59. A method according to claim 58, which comprises administering three sequential doses of the first composition, followed by one dose of the second composition.

60. A method according to claim 53, with the proviso that if the source of epitopes in (a) is a viral vector, the viral vector in (b) is derived from a different virus.

61. A method of inducing a CD4+ T-cell response against malaria in a human, which comprises the step of administering at least one dose of:
(a) a first composition comprising a source of one or more CD4+ T cell epitopes of malaria;
and at least one dose of
(b) a second composition comprising a source of one or more CD4+ T cell epitopes of malaria, including at least one CD4+ T cell epitope which is the same as a CD4+ T cell epitope of the first composition,
wherein the source of CD4+ epitopes for the first and/or second composition is a non-replicating or replication impaired recombinant poxvirus vector, and wherein the doses of the first and second compositions may be administered in either order.

62. A method inducing a combined CD4+ and CD8+ T-cell response against malaria in a human, which comprises the step of administering at least one dose of:
(a) a first composition comprising a source of one or more CD4+ T cell epitopes and a source of one or more CD8+ T cell epitopes of malaria;
and at least one dose of
(b) a second composition comprising
(i) a source of one or more CD4+ T cell epitopes of malaria, including at least one CD4+ T cell epitope which is the same as a CD4+ T cell epitope of the first composition; and
(ii) a source of one or more CD8+ T cell epitopes of the target antigen, including at least one CD8+ T cell epitope which is the same as a CD8+ T cell epitope of the first composition
wherein the source of CD4+ and CD8+ epitopes for the first and/or second composition is a non-replicating or replication impaired recombinant poxvirus vector; and wherein the doses of the first and second compositions may be administered in either order.

63. A method of boosting a primed CD4+ T cell response against at least one target antigen in a mammal, which comprises administering a source of one or more CD4+ T cell epitopes of the target antigen, wherein the source of CD4+ T cell epitopes is a non-replicating or a replication-impaired recombinant poxvirus vector.

64. A method of boosting a primed CD4+ and CD8+ T cell response against at least one target antigen in a mammal, which comprises administering a source of one or more CD4+ and CD8+ T cell epitopes of the target antigen, wherein the source of CD4+ and CD8+ T cell epitopes is anon-replicating or a replication-impaired recombinant poxvirus vector.

65. A product which comprises:
(a) a first composition comprising a source of one or more CD4+ T cell epitopes of a target antigen; and
(b) a second composition comprising a source of one or more CD4+ T cell epitopes of the target antigen, including at least one CD4+ T cell epitope which is the same as a CD4+ T cell epitope of the first composition,
wherein the source of CD4+ epitopes for the first and/or second composition is a non-replicating or replication impaired recombinant poxvirus vector; and the first composition and the second composition are a combined preparation for simultaneous, separate or sequential use for inducing a CD4+ T-cell response against a target antigen.

66. A product according to claim 65, with the proviso that if the source of epitopes in (a) is a viral vector, the viral vector in (b) is derived from a different virus.

67. A product which comprises:
(a) a first composition comprising a source of one or more CD4+ T cell epitopes and a source of one or more CD8+ T cell epitopes of a target antigen; and
(b) a second composition comprising
(i) a source of one or more CD4+ T cell epitopes of the target antigen, including at least one CD4+ T cell epitope which is the same as a CD4+ T cell epitope of the first composition; and
(ii) a source of one or more CD8+ T cell epitopes of the target antigen, including at least one CD8+ T cell epitope which is the same as a CD8+ T cell epitope of the first composition
wherein the source of CD4+ and CD8+ epitopes for the first and/or second composition is a non-replicating or replication impaired recombinant poxvirus vector; and the first composition and the second composition are a combined preparation for simultaneous, separate or sequential use for inducing a combined CD4+/CD8+ T cell immune response against the target antigen.

68. A product according to claim 67, with the proviso that if the source of epitopes in (a) is a viral vector, the viral vector in (b) is derived from a different virus.
